(19) United States
(12) Patent Application Publication
(10) Pub. No.: US 2012/0270819 A1
(43) Pub. Date: Oct. 25, 2012

(54) USE OF COMPOUNDS WITH SGLT-1/SGLT-2 INHIBITOR ACTIVITY FOR PRODUCING MEDICAMENTS FOR TREATMENT OF BONE DISEASE

(75) Inventors: Thomas Kissner, Frankfurt am Main (DE); Martin Heinrichs, Frankfurt am Main (DE); Eckart Krupp, Frankfurt am Main (DE)

(73) Assignee: SANOFI, Paris (FR)

(21) Appl. No.: 13/499,597

(22) PCT Filed: Oct. 1, 2010
(86) PCT No.: PCT/EP10/64620

§ 371 Date: Jul. 10, 2012

(30) Foreign Application Priority Data
Oct. 2, 2009 (EP) 09290759.1

(51) Int. Cl. A61K 31/706; A61P 19/02; A61K 31/70; A61P 19/10; C07H 17/02; C07H 17/00

(52) U.S. Cl. 514/27; 536/17.4; 536/18.4; 514/25

(57) ABSTRACT

Use of compounds with SGLT-1/SGLT-2 inhibitor activity for producing medicaments for treatment of bone diseases The invention relates to the use of compounds with SGLT-1/SGLT-2 inhibitor activity for producing medicaments for treatment of bone diseases like osteoporosis. Preferred is the use of compounds of the formula I in which the radicals have the stated meanings.

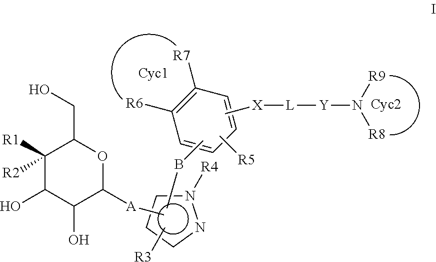

The invention relates to the use of compounds with SGLT-1/SGLT-2 inhibitor activity and of their physiologically tolerated salts and their physiologically functional derivatives for producing a medicament for treating bone diseases.

WO2004/052902, WO2004059203, disclose fluoroglycoside derivatives compounds with inhibitor activity on SGLT. These compounds are seen suitable for preventing and treating type 1 and type 2 diabetes.

We have found that these compounds exhibit SGLT-1 and SGLT-2 inhibitor activity. In WO2005121161 fluoroglycoside derivatives are described with a main inhibitor activity on SGLT-1 directed by low absorption in the intestine.

The invention was based on the object of providing compounds which can be used for the treatment of bone diseases and which are in particular therapeutically useful for treatment of osteoporosis.

The invention therefore relates to the use of compounds of formula I

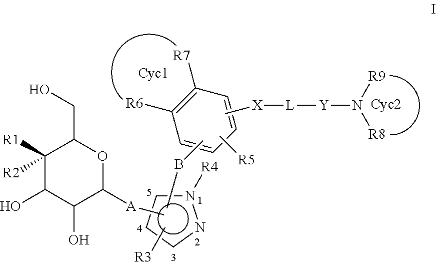

in which the meanings are
R1 and R2 independently of one another F or H, where one of the radicals R1 or R2 must be F;
A O, NH, CH₂, S or a bond;
R3 hydrogen, F, Cl, Br, I, OH, CF₃, NO₂, CN, COOH, CO—(C₁-C₆)-alkyl, COO(C₁-C₆)-alkyl, CONH₂, CONH—(C₁-C₆)-alkyl, CON[(C₁-C₆)-alkyl]₂, (C₁-C₆)-alkyl, (C₃-C₆)-cycloalkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, phenyl, benzyl, (C₁-C₆)-alkoxycarbonyl, where one, more than one or all hydrogen(s) in the alkyl, alkenyl, alkynyl and O-alkyl radicals may be replaced by fluorine;
   SO₂—NH₂, SO₂—NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o may be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH—CO—(C₁-C₇)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
R4 hydrogen, (C₁-C₆)-alkyl, (C₂-C₆)-alkenyl, (C₃-C₆)-cycloalkyl, or phenyl that may optionally be substituted by halogen or (C₁-C₄)-alkyl;
B (C₀-C₁₅)-alkylene, where one or more C atoms of the alkylene radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkylphenyl)- or —NH—;
R5, R6, R7 independently of one another, hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₈)-alkyl, HO—(C₁-C₆)-alkylene, (C₁-C₆)-alkylene-O—(C₁-C₆)-alkyl, where one, more than one, or all hydrogen(s) in the alkyl, alkenyl, alkynyl and O-alkyl radicals may be replaced by fluorine;
   SO₂—NH₂, SO₂NH(C₁-C₆)-alkyl, SO₂N[(C₁-C₆)-alkyl]₂, S—(C₁-C₆)-alkyl, 5-(CH₂)ₒ-phenyl, SCF₃, SO—(C₁-C₆)-alkyl, SO—(CH₂)ₒ-phenyl, SO₂(C₁-C₆)-alkyl, SO₂—(CH₂)ₒ-phenyl, where o may be 0-6, and the phenyl ring may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH—CO—(C₁-C₆)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₈)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
   or
R6 and R7 together with the C atoms carrying them a 5 to 7 membered, saturated, partially or completely unsaturated ring Cyc1, where 1 or 2 C atom(s) of the ring may also be replaced by N, O or S, and Cyc1 may optionally be substituted by (C₁-C₆)-alkyl, (C₂-C₆)-alkenyl, (C₂-C₆)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
X CO, O, NH, S, SO, SO₂ or a bond;
L (C₁-C₆)-alkylene, (C₂-C₅)-alkenylene, (C₂-C₅)-alkynylene, where in each case one or two CH₂ group(s) may be replaced by O or NH;
Y CO, NHCO, SO, SO₂, or a bond;
R8, R9 independently of one another, hydrogen, SO₃H, sugar residue, (C₁-C₆)-alkyl, where one or more CH₂ groups of the alkyl radical may be substituted independently of one another by (C₁-C₆)-alkyl, OH, (C₁-C₆)-alkylene-OH, (C₂-C₆)-alkenylene-OH, O-sugar residue, OSO₃H, NH₂, NH—(C₁-C₆)-alkyl, N[(C₁-C₆)-alkyl]₂, NH—CO—(C₁-C₆)-alkyl, NH-sugar residue, NH—SO₃H, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂, (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl, adamantyl; or
R8 and R9 together with the N atom carrying them form a 5 to 7 membered, saturated ring Cyc2, where one or more CH₂ groups of the ring may also be replaced by O, S, NH, NSO₃H, N-sugar residue, N—(C₁-C₆)-alkyl, where one or more CH₂ groups of the alkyl radical may be substituted independently of one another by (C₁-C₆)-alkyl, OH, (C₁-C₆)-alkylene-OH, (C₂C₆)-alkenylene-OH, NH₂, NH—(C₁-C₆)-alkyl, N[(C₁-C₆)-alkyl]₂, NH—CO—(C₁-C₆)-alkyl, NH-sugar residue, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂, (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl;

and the pharmaceutically acceptable salts thereof for producing a medicament for the treatment of bine diseases.

Sugar residues mean compounds derived from aldoses and ketoses having 3 to 7 carbon atoms, which may belong to the D or L series; also included therein are aminosaccharides, sugar alcohols or saccharic acids (Jochen Lehmann, Chemie der Kohlenhydrate, Thieme Verlag 1976). Examples which may be mentioned are glucose, mannose, fructose, galactose, ribose, erythrose, glyceraldehyde, sedoheptulose, glucosamine, galactosamine, glucuronic acid, galacturonic acid, gluconic acid, galactonic acid, mannonic acid, glucamine, 3-amino-1,2-propanediol, glucaric acid and galactaric acid. The compounds may moreover occur in the alpha and beta forms.

The points of linkage of A, B, R3 and R5 to the ring can be chosen without restriction. All resulting compounds of the formula I are included in the present invention. Preference is given to the use of compounds of the formula I in which the meanings are
A O, NH, a bond;
R3 hydrogen, F, Cl, Br, I, OH, CF₃, NO₂, CN, COOH, CO—(C₁-C₆)-alkyl, COO(C₁-C₆)-alkyl, CONH₂, CONH—(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, phenyl, benzyl, (C₁-C₄)-alkylene-COOH, SO—(C₁-C₆)-alkyl, where one, more than one or all hydrogen(s) in the alkyl radicals may be replaced by fluorine; or
R4 hydrogen, (C₁-C₆)-alkyl, (C₂-C₆)-alkenyl, (C₃-C₆)-cycloalkyl;
B (C₀-C₆)-alkylene, where one or more C atom(s) of the alkylene radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkylene-, —N((C₁-C₆)-alkylene-phenylene)- or —NH—.

Further preferred is the use of compounds of the formula I in which the sugar residues are beta(β)-linked, and the stereochemistry in the 2, 3 and 5 positions of the sugar residue has the D-gluco configuration.

Preference is further given to the use of compounds of the formula I in which
R1 is hydrogen and
R2 is fluorine;
   or
R1 is fluorine and
R2 is hydrogen;
A is O, NH;
R3 is hydrogen, F, Cl, Br, I, OH, CF₃, (C₁-C₆)-alkyl, (C₃-C₆)-cycloalkyl, (C₂-C₆)-alkenyl, O—(C₁-C₆)-alkyl, where one, more than one or all hydrogen(s) in the alkyl radicals may be replaced by fluorine;
R4 is hydrogen, (C₁-C₆)-alkyl, (C₃-C₆)-cycloalkyl;
B is (C₀-C₄)-alkylene, where one or more C atom(s) of the alkylene radical may be replaced independently of one another by —O—, —(C═O)—, —CH═CH—, —CH(OH)—, —CHF—, —CF₂— or —NH—;
R5, R6, R7 independently of one another, are hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₈)-alkyl, HO—(C₁-C₆)-alkylene, (C₁-C₆)-alkylene-O—(C₁-C₆)-alkyl, where one, more than one, or all hydrogen(s) in the alkyl, alkenyl, alkynyl and O-alkyl radicals may be replaced by fluorine;
   NH₂, NH—(C₁-C₆)-alkyl, N((C₁-C₆)-alkyl)₂, NH—CO—(C₁-C₆)-alkyl,
   or
R6 and R7 together with the C atoms carrying them are a 5 to 7 membered, saturated, partially or completely unsaturated ring Cyc1, where 1 or 2 C atom(s) of the ring may also be replaced by N, O or S, and Cyc1 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
X is CO, O, NH, a bond;
L is (C₁-C₆)-alkylene, (C₂-C₅)-alkenylene, where in each case one or two CH₂ group(s) may be replaced by O or NH;
Y is CO, NHCO, a bond.

Particular preference is given to the use of compounds of the formula I in which
R1 is hydrogen;
R2 is fluorine;
A is O;
R3 is CF₃, methyl, isopropyl;
R4 is hydrogen;
B is (C₀-C₄)-alkylene, where one or more C atom(s) of the alkylene radical may be replaced independently of one another by —O—, —(C═O)—, —CHF— or —CF₂—;
X is CO, 0, a bond;
L is (C₁-C₄)-alkylene, (C₂-C₄)-alkenylene, where in each case one or two CH₂ group(s) may be replaced by O or NH;
Y is CO, NHCO, a bond.

Very particular preference is given to the use of compounds of the formula I in which
R1 is hydrogen;
R2 is fluorine;
A is O;
B is —CH₂—;
R5 is hydrogen, Cl, methyl, ethyl, OH, CF₃;
R6, R7 are hydrogen;
X is CO, 0, a bond;
L is (C₁-C₃)-alkylene, (C₂-C₃)-alkenylene, where in each case one CH₂ group may be replaced by O or NH;
Y is CO, NHCO, a bond.

Particularly preferred is the u se of compounds of the formula I in which the substituents A and B occupy an adjacent position (ortho position) and R3 occupies an adjacent position (ortho position) to B.

Very particular preference is further given to the use of compounds of the formula I in which
R8, R9 independently of one another, are hydrogen, SO₃H, sugar residue, (C₁-C₄)-alkyl, where the alkyl radical may be substituted independently of one another one or more times by (C₁-C₂)-alkyl, OH, (C₁-C₂)-alkylene-OH, OSO₃H, NH₂, CONH₂, SO₂NH₂, NH—SO₃H or adamantyl; or
R8 and R9 together with the N atom carrying them form a 5 to 7 membered, saturated ring Cyc2, selected from the group of piperazine which may be N-substituted by (C₁-C₂)-alkyl, (C₁-C₂)-alkylene-OH or SO₃H, piperidine, azepane, pyrrolidine or morpholine.

In a particular embodiment the compounds of the formula I, the substituents B and X are disposed in para position on the phenyl ring.

In a further embodiment the compounds of formula I, the substituents A are disposed in position 3, B in position 4 and R3 in position 5 on the pyrazole ring.

In a further embodiment the compounds of formula I, the substituents A are disposed in position 5, B in position 4 and R3 in position 3 on the pyrazole ring.

The alkyl radicals in the substituents R3, R4, R5, R6, R7, R8 and R9 may be either straight-chain or branched. Halogen means F, Cl, Br, I, preferably F and Cl.

The invention also relates to the use of compounds of the formula II

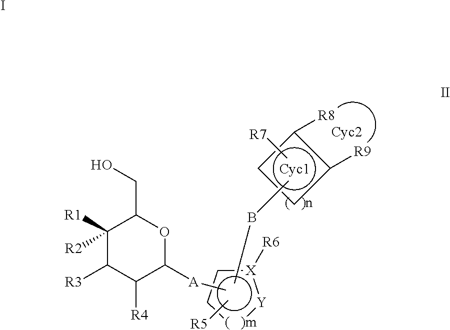

in which the meanings are
R1 and R2 independently of one another F, H or one of the radicals R1 or R2 OH;
R3 OH or F, where at least one of the radicals R1, R2, R3 must be F;
R4 OH;
A O, NH, CH₂, S or a bond;
X C, O, S or N, where X must be C when Y is O or S;
Y N, O or S;
m a number 1 or 2;
R5 hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₆)-alkoxycarboxyl, it being possible for one, more than one or all hydrogen(s) in the alkyl, alkoxy, alkenyl or alkynyl radicals to be replaced by fluorine;
   SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
   or when Y is S, R5 and R6 together with the C atoms carrying them phenyl;
R6 optionally H, (C₁-C₆)-alkyl, (C₁-C₆)-alkenyl, (C₃-C₆)-cycloalkyl, or phenyl that may optionally be substituted by halogen or (C₁-C₄)-alkyl;
B (C₀-C₁₅)-alkanediyl, it being possible for one or more C atoms in the alkanediyl radical to be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, it being possible for one, more than one or all hydrogen(s) in the alkyl, alkoxy, alkenyl or alkynyl radicals to be replaced by fluorine;
   SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO(C₁-C₆)-alkyl, CONH₂;
   or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
and the pharmaceutically acceptable salts thereof for producing a medicament for the treatment of bone diseases.

The points of linkage of A, B and R₅ to the ring can be chosen without restriction. The present invention includes the use of all the resulting compounds of the formula II.

Suitable heterocycles of the central building block comprising X and Y are: thiophene, furan, pyrrole, pyrazole, isoxazole and isothiazole, with preference for thiophene, pyrazole and isoxazole. Particularly preferred compounds of the formula II are those comprising thiophene or pyrazole as central building block.

Preferred is the use of compounds of the formula II in which the meanings are
R1 and R2 independently of one another F or H and one of the radicals R1 or R2=OH, where one of the radicals R1 or R2 must be F;
R3 OH;
R4 OH;
A O or NH;
X C, O or N, where X must be C when Y is S;
Y S or N;
m a number 1 or 2;
R5 hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₄)-alkylcarboxyl, SO—(C₁-C₆)-alkyl, it being possible for one, more than one or all hydrogen(s) in the alkyl or alkoxy radicals to be replaced by fluorine; or
   when Y is S, R5 and R6 together with the C atoms carrying them phenyl;
R6 optionally H, (C₁-C₆)-alkyl, (C₁-C₆)-alkenyl, (C₃-C₆)-cycloalkyl, or phenyl that may optionally be substituted by halogen or (C₁-C₄)-alkyl;
B (C₀-C₁₅)-alkanediyl, where one or more C atom(s) in the alkanediyl radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, S—(C₁-C₆)-alkyl, SCF₃, SO—(C₁-C₆)-alkyl, it being possible for one, more than one or all hydrogen(s) in the alkyl or alkoxy radicals to be replaced by fluorine;
   or
R8 and R9 together with the C atoms carrying them a 5 to 7 membered, saturated, partially or completely unsaturated ring Cyc2, where 1 or 2 C atom(s) in the ring may also be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃.

Further preferred is the use of compounds of the formula II in which the sugar residues are beta(β)-linked and the stereochemistry in the 2, 3 and 5 position of the sugar residue has the D-gluco configuration.

Particularly preferred is the use of compounds of the formula II in which the substituents A and B occupy an adjacent position (ortho position).

Particularly preferred is the use of compounds of the formula II in which
R1 and R2 are independently of one another F, H or one of the radicals R1 or R2=OH where at least one of the radicals R1 or R2 must be F;
R3 is OH
R4 is OH;
A is O;
X is C, O or N, where X must be C when Y is S;
Y is S or N;
m is a number 1;
R5 is hydrogen, (C₁-C₅)-alkyl, (C₁-C₄)-alkoxy, HO—(C₁-C₄)-alkyl, (C₁-C₄)-alkyl-O—(C₁-C₄)-alkyl, F, Cl, CF₃, OCF₃, OCH₂CF₃ (C₁-C₄)-alkyl-CF₂—, phenyl, benzyl, (C₁-C₄)-alkylcarboxyl, (C₂-C₄)-alkenyl, (C₂-C₄)-alkynyl, COO(C₁-C₄)-alkyl; or
   when Y is S, R5 and R6 together with the C atoms carrying them phenyl;
R6 is optionally H, (C₁-C₆)-alkyl, (C₁-C₆)-alkenyl, (C₃-C₆)-cycloalkyl, or phenyl that may optionally be substituted by halogen or (C₁-C₄)-alkyl;
B is (C₁-C₄)-alkanediyl, where one CH₂ group may also be replaced by —(C═O)—, —CH(OH)—, —CO—NH—, —CHF—, —CF₂—, —O—;
n is a number 2 or 3;
Cyc1 is unsaturated 5- or 6-membered ring, where 1 C atom may be replaced by O or S;
R7, R8, R9 are hydrogen, (C₁-C₄)-alkyl, (C₁-C₈)-alkoxy, S—(C₁-C₄)-alkyl, SCF₃, F, Cl, Br, I, OCF₃, OCH₂CF₃, OH, HO—(C₁-C₄)-alkyl, (C₁-C₄)-alkyl-O—(C₁-C₄)-alkyl, or
R8 and R9 together are —CH═CH—O—, —CH═CH—S—, CH═CH—CH═CH—, which is optionally substituted by (C₁-C₄)-alkoxy, or —O—(CH₂)ₚ—O—, with p=1 or 2 and
R7 is hydrogen.

Very particularly preferred is the use of compounds of the formula in which
R1, R2 are H or F, where one of the radicals R1, R2 must be F;
R3 is OH;
R4 is OH;
A is O;
X is C and Y is S, or
X is O and Y is N, or
X is N and Y is N;
m is a number 1;
R5 is hydrogen, CF₃, (C₁-C₆)-alkyl, or when Y is S R5 and R6 together with the C atoms carrying them are phenyl;
R6 is optionally H, (C₁-C₄)-alkyl or phenyl;
B is —CH₂—, —C₂H₄—, —C₃H₆, —CO—NH—CH₂— or —CO—CH₂—CH₂—;
n is a number 2 or 3;
Cyc1 is an unsaturated 5 to 6 membered ring, where 1 C atom can be replaced by S;
R7, R8, R9 are hydrogen, (C₁-C₆)-alkyl, (C₁-C₄)-alkoxy, S—(C₁-C₄)-alkyl, SCF₃, F, Cl, Br, I, OCF₃, or
R8 and R9 together are —CH═CH—O—, —CH═CH—CH═CH—, which is optionally substituted by (C₁-C₄)-alkoxy, and
R7 is hydrogen.

Further very particularly preferred is the use of compounds of the formula II in which
R1, R2 are H or F, where one of the radicals R1 or R2 is F;
R3 is OH;
R4 is OH;
A is O;
X is C and Y is S or
X is N and Y is N;
m is a number 1;
R5 is hydrogen, (C₁-C₄)-alkyl or CF₃ or when Y is S R5 and R6 together with the carbon atoms carrying them are phenyl;
R6 is optionally H or (C₁-C₄)-alkyl;
B is —CH₂— or —CO—NH—CH₂—;
n is a number 2 or 3;
Cyc1 is phenyl or thiophene;
R7 is hydrogen, methoxy, F, Cl, Br, I, (C₁-C₄)-alkyl, OCF₃;
R8, R9 are hydrogen or Cl or
R8 and R9 together with the carbon atoms carrying them are phenyl which may optionally be substituted by methoxy, or furan and
R7 is hydrogen.

The linkage of one of the substituents A or B particularly preferably takes place in a position adjacent to the variable Y.

Additional very particularly preferred compounds which may be mentioned are those in which Y is S and those in which R1 is H and R2 is F.

The invention relates to the use of compounds of the formula II in the form of their racemates, racemic mixtures and pure enantiomers and to their diastereomers and mixtures thereof.

The alkyl radicals in the substituents R4, R5, R6, R7, R8 and R9 may be either straight-chain or branched. Halogen means F, Cl, Br, I, preferably F or Cl.

The invention also relates to the use of compounds of the formula III

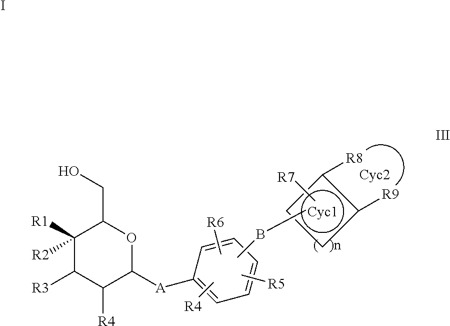

in which the meanings are
R1, R2 OH, F or H or R1 and R2=F, excluding the three combinations R1=F, R2=OH and R1=OH, R2=F and R1, R2=OH;
R3 OH or F, where at least one of the R1, R2, R3 radicals must be F;
A O, NH, CH₂, S or a bond;
R4, R5, R6 hydrogen, F, Cl, Br, I, OH, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO(C₁-C₆)-alkyl, (C₁-C₆)-alkyl-O—(C₁-C₆)-alkyl, phenyl, benzyl, it being possible for one, more than one or all hydrogen(s) in the alkyl, alkoxy, alkenyl and alkynyl radicals to be replaced by fluorine;
   SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
B (C₀-C₁₅)-alkanediyl, it being possible for one or more C atoms in the alkanediyl radical to be replaced independently of one another by —O—, —(C═O)—, —CH═CH—, —C≡C-—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, (C₁-C₆)-alkyl-OH, (C₁-C₆)-alkyl-O—(C₁-C₆)-alkyl, it being possible for one, more than one or all hydrogen(s) in the alkyl radicals to be replaced by fluorine;
   SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
   NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
   or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₆)-alkenyl, (C₂-C₆)-alkynyl, where in each case one CH₂ group may be replaced by O, or substituted by H, F, Cl, OH, CF₃, NO₂, CN, COO—(C₁-C₄)-alkyl, CONH₂, CONH(C₁-C₄)-alkyl, OCF₃;

and the pharmaceutically acceptable salts thereof for producing a medicament for the treatment of bone diseases.

The linkage points of R4, R5, R6 and B to the phenyl ring can be freely selected. Compounds of the formula III in which the B substituent on the phenyl ring is disposed in the position ortho (neighboring position) to the A substituent are preferred.

Preferred is the use of compounds of the formula III in which the meanings are
R1, R2 OH, F or H or R1 and R2=F, where one of the radicals R1 or R2 must be F, excluding the combinations R1=F, R2=OH and R1=OH, R2=F and R1, R2=OH;
R3 OH;
A O or NH;
R4, R5, R6 hydrogen, F, Cl, Br, I, OH, CF₃, NO₂, CN, COON, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO(C₁-C₆)-alkyl, (C₁-C₆)-alkyl-O—(C₁-C₆)-alkyl, phenyl, benzyl, SO—(C₁-C₆)-alkyl, it being possible for one, more than one or all hydrogen(s) in the alkyl, alkoxy, alkenyl and alkynyl radicals to be replaced by fluorine;
B (C₀-C₁₅)-alkanediyl, where one or more C atom(s) in the alkanediyl radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, (C₁-C₆)-alkyl-OH, (C₁-C₆)-alkyl-O—(C₁-C₆)-alkyl, SO—(C₁-C₆)-alkyl, it being possible for one, more than one or all hydrogen(s) in the alkyl radicals to be replaced by fluorine;
   or
R8 and R9 together with the C atoms carrying them a 5 to 7 membered, saturated, partially or completely unsaturated ring Cyc2, where 1 or 2 C atom(s) in the ring may also be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃.

Further preferred is the use of compounds of the formula III in which the sugar residues are beta(β)-linked and the stereochemistry in the 2, 3 and 5 position of the sugar residue has the D-gluco configuration.

Particularly preferred is the use of compounds of the formula III in which
R1, R2 are OH, F or H or R1 and R2=F, where one of the radicals R1 or R2 must be F, excluding the combinations R1=F, R2=OH and R1=OH, R2=F and R1, R2=OH;
R3 is OH;
A is O;
R4, R5, R6 are hydrogen, OH, (C₁-C₆)-alkyl, (C₁-C₄)-alkoxy, HO—(C₁-C₄)-alkyl, (C₁-C₄)-alkyl-O—(C₁-C₄)-alkyl, F, Cl, Br, I, CF₃, OCF₃, OCH₂CF₃ (C₁-C₄)-alkyl-CF₂—, phenyl, benzyl, (C₂-C₄)-alkenyl, (C₂-C₄)-alkynyl, COO(C₁-C₄)-alkyl;
B is (C₁-C₄)-alkanediyl, where one CH₂ group may also be replaced by —(C═O)—, —CH(OH)—, —CO—NH—, —CO—N(C₁-C₆)-alkyl-, —CHF—, —CF₂—, —O—, —NH—;
n is a number 2 or 3;
Cyc1 is unsaturated 5- or 6-membered ring, where 1 C atom may be replaced by O, N or S;
R7, R8, R9 are hydrogen, (C₁-C₆)-alkyl, (C₁-C₈)-alkoxy, OCF₃, OCH₂CF₃, OH, (C₁-C₄)-alkyl-OH, (C₁-C₄)-alkyl-O—(C₁-C₄)-alkyl, F, Cl, Br or
R8 and R9 together are —CH═CH—O—, —CH₂—CH₂—O—, —CH═CH—S—, —CH═CH—CH═CH—, —O—(CH₂)ₚ—O—, with p=1 or 2, and
R7 is methyl, ethyl, OMe, F, Cl, Br or hydrogen.

Very particularly preferred is the use of compounds of the formula III in which
R1 is F and R2 is H or
R1 is H and R2 is F;
R1 is F and R2 is F
R3 is OH;
A is O;
R4, R5, R6 are hydrogen, OH, (C₁-C₄)-alkoxy, CF₃, (C₁-C₄)-alkyl, F, Cl, Br, I
B is —CH₂—, —C₂H₄—, —C₃H₆—, —CH(OH)—, —(C═O)—, —CO—NH—CH₂— or —CO—CH₂—CH₂—, —O—, —NH—;
n is a number 2 or 3;
Cyc1 is unsaturated 6-membered ring, where 1 C atom may be replaced by N, or unsaturated 5-membered ring, where 1 C atom may be replaced by S;
R7, R8, R9 are hydrogen, OH, (C₁-C₄)-alkyl, (C₁-C₇)-alkoxy, OCF₃, halogen or
R8 and R9 together are —CH═CH—O—, —CH₂—CH₂—O—, —CH═CH—CH═CH—, —O—(CH₂)ₚ—O—, with p=1 or 2, and
R7 is methyl, ethyl, methoxy, F, Cl, Br, hydrogen.

Further very particularly preferred is the use of compounds of the formula IIIa

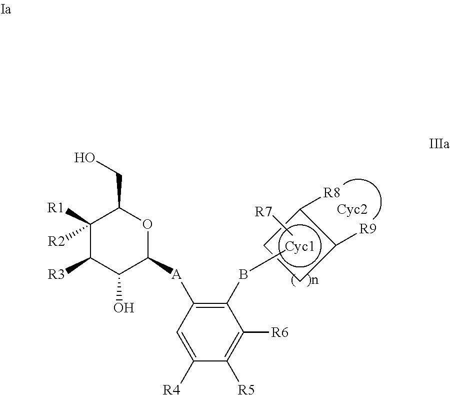

in which
R1 is F and R2 is H or
R1 is H and R2 is F or
R1 is F and R2 is F;
R3 is OH;
A is O;
R4 is hydrogen, (C₁-C₄)-alkyl, (C₁-C₄)-alkoxy or OH;
R5 is hydrogen, F, methoxy or ethoxy;
R6 is hydrogen or OH;
B is —CH₂—, —CO—NH—CH₂—; —O— or —CO—CH₂—CH₂—;
Cyc1 is phenyl or thiophene;
R7, R8, R9 are hydrogen, OH, Cl, OCF₃, (C₁-C₄)-alkyl or (C₁-C₄)-alkoxy; or
R8 and R9 together are —CH═CH—O—, —CH═CH—CH═CH— or —CH₂—CH₂—O— and
R7 is hydrogen.

The use of compounds of particularly preferred importance are also those of the formula IIIb

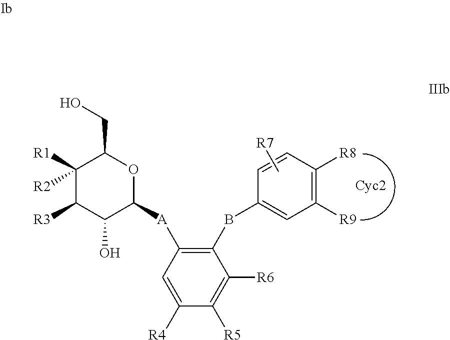

in which
R1 is F and R2 is H or
R1 is H and R2 is F or
R1 is F and R2 is F;
R3 is OH;
A is O;
R4 is hydrogen, methyl, methoxy or OH;
R5 is hydrogen, F or methoxy;
R6 is hydrogen or OH;
B is —CH₂—, —CO—NH—CH₂—; —O— or —CO—CH₂—CH₂—;
Cyc1 is phenyl;
R7 is hydrogen;
R8 is hydrogen, OH, ethyl, Cl, OCF₃ or methoxy;
R9 is hydrogen; or
R8 and R9 together are —CH═CH—O— or —CH₂—CH₂—O—.

Additional very particularly preferred is the use of compounds of the formula III are those in which R1 is H and R2 is F.

The alkyl radicals in the substituents R4, R5, R6, R7, R8 and R9 may be either straight-chain or branched. Halogen means F, Cl, Br or I, preferably F or Cl.

The invention relates to compounds of the formula I, II and III in the form of their tautomers, racemates, racemic mixtures and pure enantiomers, and to their diastereomers and mixtures thereof. The present invention includes all these isomeric and, where appropriate, tautomeric forms of the compounds of the formula I, II and III. These isomeric forms can be obtained by known methods even if not (in some cases) expressly described.

Pharmaceutically acceptable salts are, because their solubility in water is greater than that of the starting or basic compounds, particularly suitable for medical applications. These salts must have a pharmaceutically acceptable anion or cation. Suitable pharmaceutically acceptable acid addition salts of the compounds of the invention are salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic and tartaric acid. Suitable pharmaceutically acceptable basic salts are ammonium salts, alkali metal salts (such as sodium and potassium salts), alkaline earth metal salts (such as magnesium and calcium salts) and salts of trometamol (2-amino-2-hydroxymethyl-1,3-propanediol), diethanolamine, lysine or ethylenediamine.

Salts with a pharmaceutically unacceptable anion such as, for example, trifluoroacetate likewise belong within the framework of the invention as useful intermediates for the preparation or purification of pharmaceutically acceptable salts and/or for use in nontherapeutic, for example in vitro, applications.

The term “physiologically functional derivative” used herein refers to any physiologically tolerated derivative of a compound of the formula I, II and III of the invention, for example an ester, which on administration to a mammal such as, for example, a human is able to form (directly or indirectly) a compound of the formula I, II and III or an active metabolite thereof.

Physiologically functional derivatives include prodrugs of the compounds of the invention, as described, for example, in H. Okada et al., Chem. Pharm. Bull. 1994, 42, 57-61. Such prodrugs can be metabolized in vivo to a compound of the invention. These prodrugs may themselves be active or not. Carbonates at the 6 position of the sugar (see WO 0280936 and WO 0244192) are preferred, particularly preferably methyl carbonate and ethyl carbonate.

The compounds of the invention may also exist in various polymorphous forms, for example as amorphous and crystalline polymorphous forms. All polymorphous forms of the compounds of the invention belong within the framework of the invention and are a further aspect of the invention.

All references to “compound(s) of formula I, II and III” hereinafter refer to compound(s) of the formula I, II and III as described above, and their salts, solvates and physiologically functional derivatives as described herein.

Use

The compounds of the formula I, II and III are distinguished by beneficial effects on glucose metabolism; in particular, they lower the blood glucose level and are suitable for the treatment of type 1 and type 2 diabetes. The compounds can therefore be employed alone or in combination with other blood glucose-lowering active ingredients (antidiabetics).

The compounds of the formula I, II and III are further suitable for the prevention and treatment of late damage from diabetes, such as, for example, nephropathy, retinopathy, neuropathy and syndrome X, obesity, myocardial infarction, myocardial infarct, peripheral arterial occlusive diseases, thromboses, arteriosclerosis, inflammations, immune diseases, autoimmune diseases such as, for example, AIDS, asthma, osteoporosis, cancer, psoriasis, Alzheimer's, schizophrenia and infectious diseases, preference being given to the treatment of type 1 and type 2 diabetes and for the prevention and treatment of late damage from diabetes, syndrome X and obesity.

Unexpected results from animal studies corroborate also beneficial effects on bone parameter for compounds of SGLT-1/SGLT-2 inhibitor activity, like the compounds of formula I, II and III.

The compounds may decrease consequently the bone turnover which resulted in positive effects on bone mass and bone strength associated parameters. Compounds of SGLT-1/SGLT-2 inhibitor activity, like the compounds of formula I, II and III are therefore suitable for the prevention and/or treatment of bone diseases like osteoporosis, osteolysis or aseptic loosening in joint implants, preferred use is the prevention and/or treatment of osteoporosis.

Formulations

The amount of a compound of formula I, II and III necessary to achieve the desired biological effect depends on a number of factors, for example the specific compound chosen, the intended use, the mode of administration and the clinical condition of the patient. The daily dose is generally in the range from 0.3 mg to 100 mg (typically from 3 mg and 50 mg) per day and per kilogram of bodyweight, for example 3-10 mg/kg/day. Single-dose formulations which can be administered orally, such as, for example, tablets or capsules, may contain, for example, from 1.0 to 1000 mg, typically from 10 to 600 mg. For the therapy of the abovementioned conditions, the compounds of formula I, II and III may be used as the compound itself, but they are preferably in the form of a pharmaceutical composition with an acceptable carrier. The carrier must, of course, be acceptable in the sense that it is compatible with the other ingredients of the composition and is not harmful for the patient's health. The carrier may be a solid or a liquid or both and is preferably formulated with the compound as a single dose, for example as a tablet, which may contain from 0.05% to 95% by weight of the active ingredient. Other pharmaceutically active substances may likewise be present, including other compounds of formula I, II and III. The pharmaceutical compositions of the invention can be produced by one of the known pharmaceutical methods, which essentially consist of mixing the ingredients with pharmacologically acceptable carriers and/or excipients.

Pharmaceutical compositions of the invention are those suitable for oral, rectal, topical, peroral (for example sublingual) and administration, although the most suitable mode of administration depends in each individual case on the nature and severity of the condition to be treated and on the nature of the compound of formula I, II and III used in each case. Coated formulations and coated slow-release formulations also belong within the framework of the invention. Preference is given to acid- and gastric juice-resistant formulations. Suitable coatings resistant to gastric juice comprise cellulose acetate phthalate, polyvinyl acetate phthalate, hydroxypropylmethylcellulose phthalate and anionic polymers of methacrylic acid and methyl methacrylate.

Suitable pharmaceutical compounds for oral administration may be in the form of separate units such as, for example, capsules, cachets, suckable tablets or tablets, each of which contain a defined amount of the compound of formula I, II and III; in the form of powders or granules; as solution or suspension in an aqueous or nonaqueous liquid; or in the form of an oil-in-water or water-in-oil emulsion. These compositions may, as already mentioned, be prepared by any suitable pharmaceutical method which includes a step in which the active ingredient and the carrier (which may consist of one or more additional ingredients) are brought into contact. The compositions are generally produced by uniform and homogeneous mixing of the active ingredient with a liquid and/or finely divided solid carrier, after which the product is shaped if necessary. Thus, for example, a tablet can be produced by compressing or molding a powder or granules of the compound, where appropriate with one or more additional ingredients. Compressed tablets can be produced by tableting the compound in free-flowing form such as, for example, a powder or granules, where appropriate mixed with a binder, glidant, inert diluent and/or one (or more) surface-active/dispersing agent(s) in a suitable machine. Molded tablets can be produced by molding the compound, which is in powder form and is moistened with an inert liquid diluent, in a suitable machine.

Pharmaceutical compositions which are suitable for peroral (sublingual) administration comprise suckable tablets which contain a compound of formula I, II and III with a flavoring, normally sucrose and gum arabic or tragacanth, and pastilles which comprise the compound in an inert base such as gelatin and glycerol or sucrose and gum arabic.

Pharmaceutical compositions suitable for parenteral administration comprise preferably sterile aqueous preparations of a compound of formula I, II and III, which are preferably isotonic with the blood of the intended recipient. These preparations are preferably administered intravenously, although administration may also take place by subcutaneous, intramuscular or intradermal injection. These preparations can preferably be produced by mixing the compound with water and making the resulting solution sterile and isotonic with blood. Injectable compositions of the invention generally contain from 0.1 to 5% by weight of the active compound.

Pharmaceutical compositions suitable for rectal administration are preferably in the form of single-dose suppositories. These can be produced by mixing a compound of the formula I, II and III with one or more conventional solid carriers, for example cocoa butter, and shaping the resulting mixture.

Pharmaceutical compositions suitable for topical use on the skin are preferably in the form of ointment, cream, lotion, paste, spray, aerosol or oil. Carriers which can be used are petrolatum, lanolin, polyethylene glycols, alcohols and combinations of two or more of these substances. The active ingredient is generally present in a concentration of from 0.1 to 15% by weight of the composition, for example from 0.5 to 2%.

Transdermal administration is also possible. Pharmaceutical compositions suitable for transdermal uses can be in the form of single plasters which are suitable for long-term close contact with the patient's epidermis. Such plasters suitably contain the active ingredient in an aqueous solution which is buffered where appropriate, dissolved and/or dispersed in an adhesive or dispersed in a polymer. A suitable active ingredient concentration is about 1% to 35%, preferably about 3% to 15%. A particular possibility is for the active ingredient to be released by electrotransport or iontophoresis as described, for example, in Pharmaceutical Research, 2(6): 318 (1986).

The preparation of the examples is described in detail below. The compounds of formula I can be obtained analogously or in accordance with the processes described in WO 0414932 and WO 0418491.

Compounds of the general formula II can be obtained as shown in the following reaction schemes for processes A, B and C;

Process A:

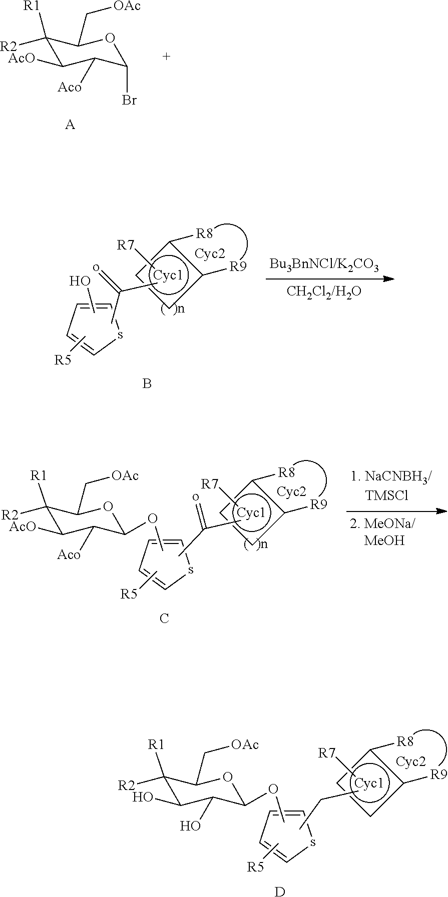

Process B:

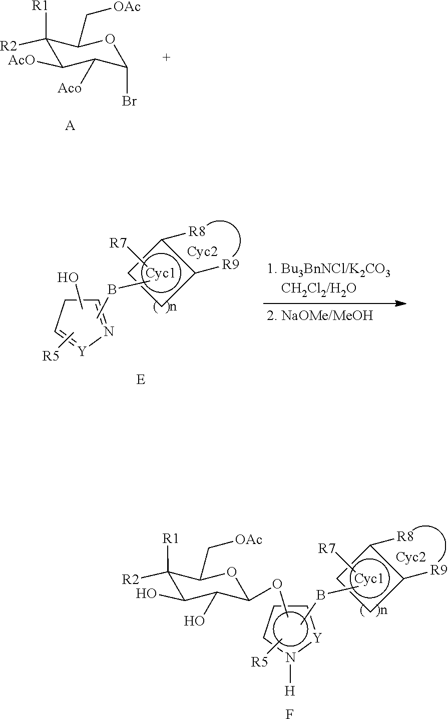

Process C:

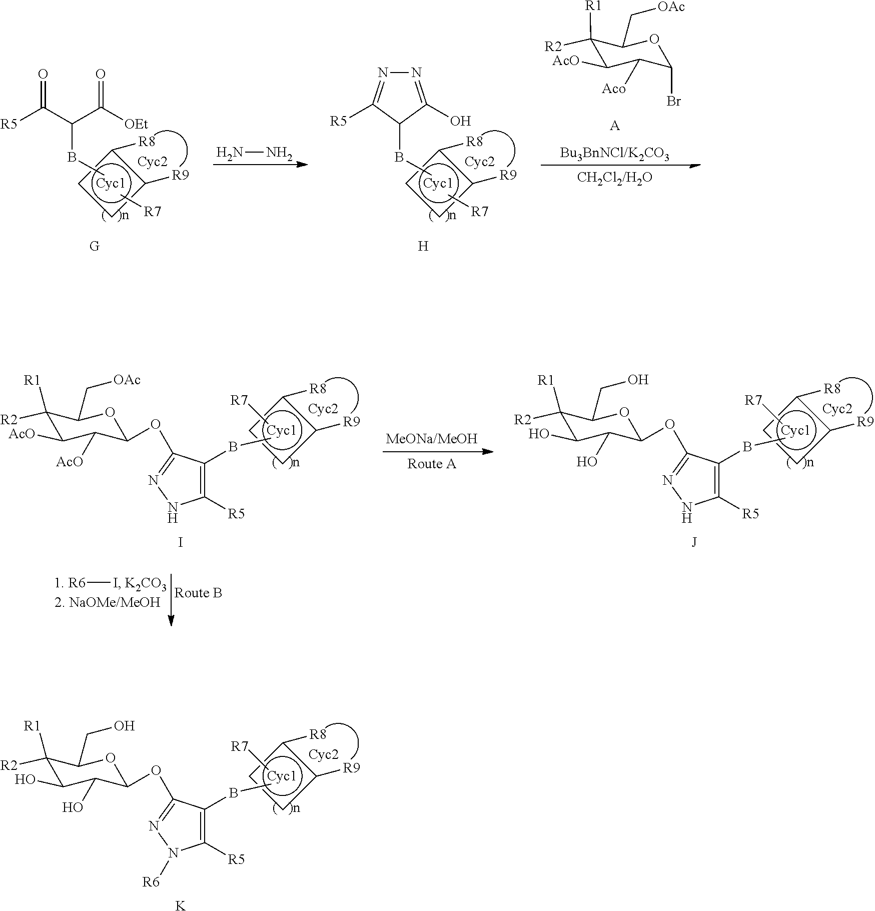

The schemes depicted for processes A, B and C are self-explanatory and can be carried out thus by the skilled worker. More details are, nevertheless, indicated in the experimental part. The compounds of examples 1 to 31 were obtained by processes A, B and C. Other compounds of the formula II can be obtained correspondingly or by known processes.

Compounds of the formula III can be obtained in accordance with the following reaction schemes of processes A3 to F3.

Process A3:

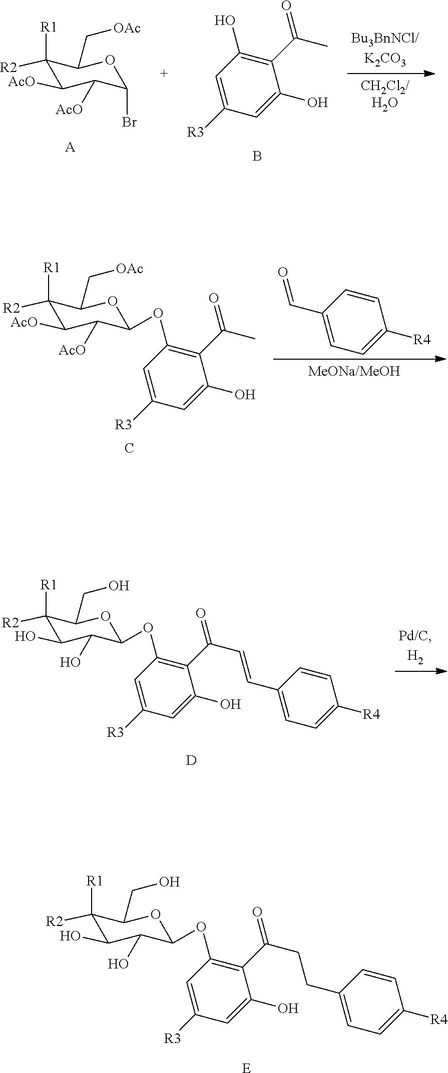

Process B3:

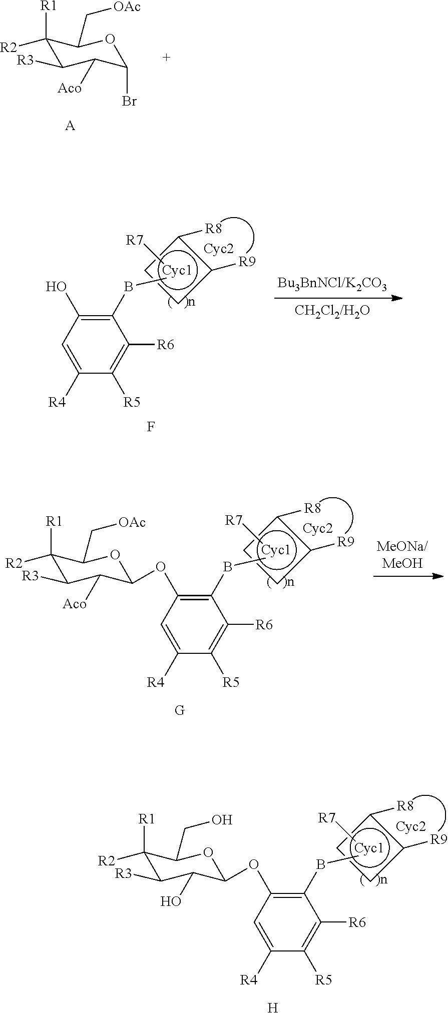

Process C3:

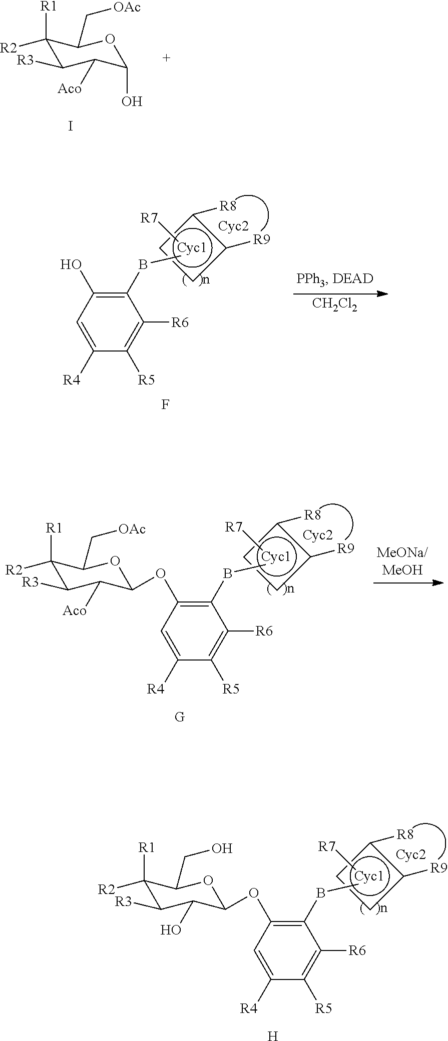

Process D3:

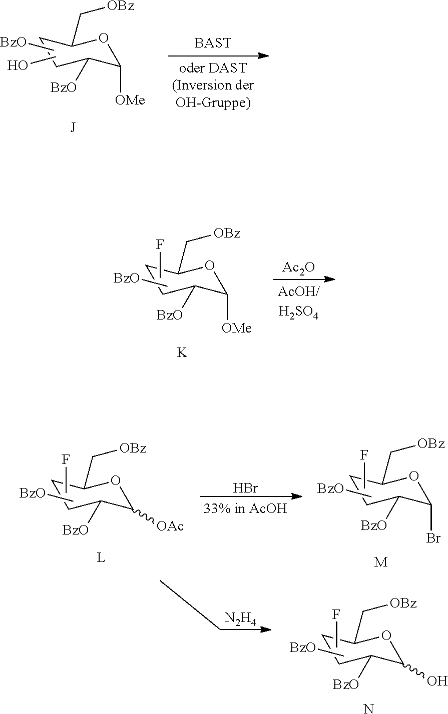

Process E3:

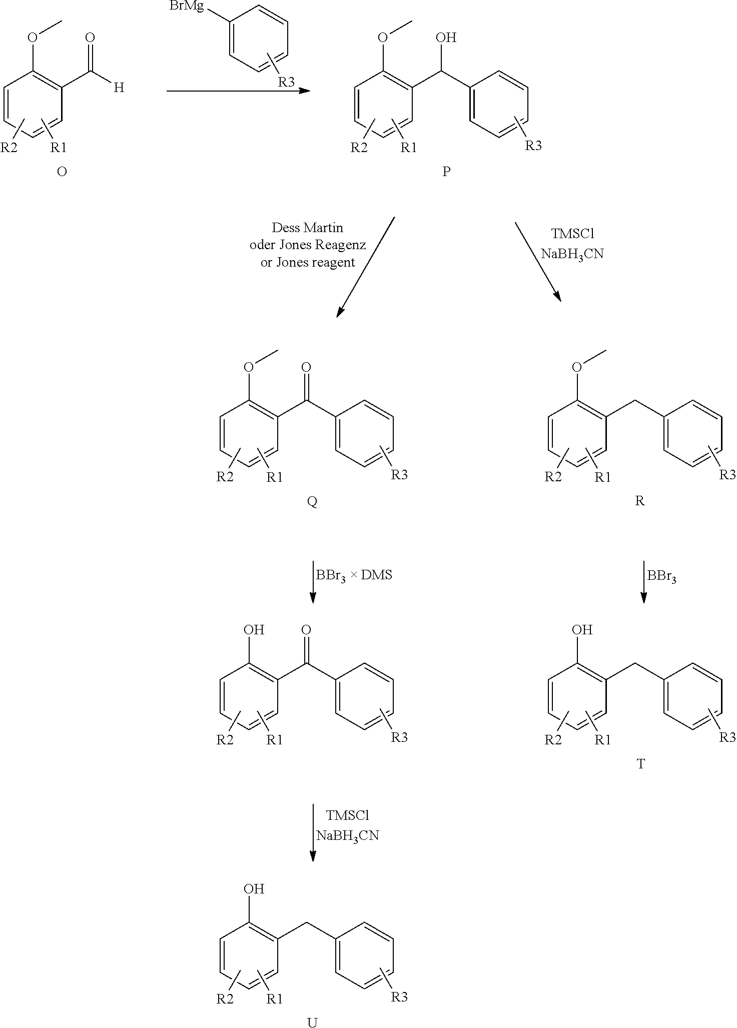

Verfahren F3:

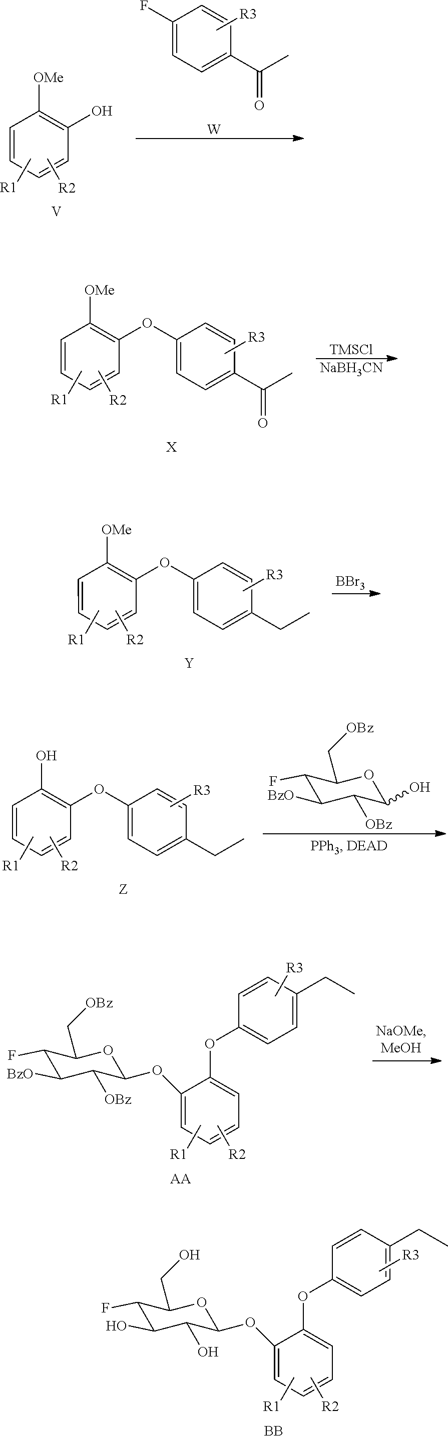

The schemes depicted for processes A3-F3 are self-explanatory and can be carried out thus by the skilled worker. More details are, nevertheless, indicated in the experimental part. The compounds of examples 1 to 24 were obtained by processes A3-F3. Other compounds of the formula III can be obtained correspondingly or by known processes.

The compound(s) of the formula I, II and III can also be administered in combination with other active ingredients.

Further active ingredients suitable for combination products are:

all antidiabetics mentioned in the Rote Liste 2001, chapter 12. They may be combined with the compounds of the formula I, II and III of the invention in particular for synergistic improvement of the effect. Administration of the active ingredient combination may take place either by separate administration of the active ingredients to the patient or in the form of combination products in which a plurality of active ingredients are present in one pharmaceutical preparation. Most of the active ingredients listed below are disclosed in the USP Dictionary of USAN and International Drug Names, US Pharmacopeia, Rockville 2001.

Antidiabetics include insulin and insulin derivatives such as, for example, Lantus® (see www.lantus.com) or HMR 1964, fast-acting insulins (see U.S. Pat. No. 6,221,633), GLP-1 derivatives such as, for example, those disclosed in WO 98/08871 of Novo Nordisk A/S, and orally effective hypoglycemic active ingredients.

The orally effective hypoglycemic active ingredients include, preferably, sulfonylureas, biguanidines, meglitinides, oxadiazolidinediones, thiazolidinediones, glucosidase inhibitors, glucagon antagonists, GLP-1 agonists, potassium channel openers such as, for example, those disclosed in WO 97/26265 and WO 99/03861 of Novo Nordisk A/S, insulin sensitizers, inhibitors of liver enzymes involved in the stimulation of gluconeogenesis and/or glycogenolysis, modulators of glucose uptake, compounds which alter lipid metabolism, such as antihyperlipidemic active ingredients and antilipidemic active ingredients, compounds which reduce food intake, PPAR and PXR agonists and active ingredients which act on the ATP-dependent potassium channel of the beta cells.

In one embodiment of the invention, the compounds of the formula I, II and III are administered in combination with an HMGCoA reductase inhibitor such as simvastatin, fluvastatin, pravastatin, lovastatin, atorvastatin, cerivastatin, rosuvastatin.

In one embodiment of the invention, the compounds of the formula I, II and III are administered in combination with a cholesterol absorption inhibitor such as, for example, ezetimibe, tiqueside, pamaqueside.

In one embodiment of the invention, the compounds of the formula I, II and III are administered in combination with a PPAR gamma agonist, such as, for example, rosiglitazone, pioglitazone, JTT-501, GI 262570.

In one embodiment of the invention, the compounds of the formula I, II and III are administered in combination with a PPAR alpha agonist, such as, for example, GW 9578, GW 7647.

In one embodiment of the invention, the compounds of the formula I, II and III are administered in combination with a mixed PPAR alpha/gamma agonist, such as, for example, GW 1536, AVE 8042, AVE 8134, AVE 0847, AVE 0897 or as described in WO 00/64888, WO 00/64876, WO 03/20269.

In one embodiment of the invention, the compounds of the formula I, II and III are administered in combination with a fibrate such as, for example, fenofibrate, clofibrate, bezafibrate.

In one embodiment of the invention, the compounds of the formula I, II and III are administered in combination with an MTP inhibitor such as, for example, implitapide, BMS-201038, R-103757.

In one embodiment of the invention, the compounds of the formula I, II and III are administered in combination with bile acid absorption inhibitor (see, for example, U.S. Pat. No. 6,245,744 or U.S. Pat. No. 6,221,897), such as, for example, HMR 1741.

In one embodiment of the invention, the compounds of the formula I, II and III are administered in combination with a CETP inhibitor, such as, for example, JTT-705.

In one embodiment of the invention, the compounds of the formula I, II and III are administered in combination with a polymeric bile acid adsorbent such as, for example, cholestyramine, colesevelam.

In one embodiment of the invention, the compounds of the formula I, II and III are administered in combination with an LDL receptor inducer (see U.S. Pat. No. 6,342,512), such as, for example, HMR1171, HMR1586.

In one embodiment of the invention, the compounds of the formula I, II and III are administered in combination with an ACAT inhibitor, such as, for example, avasimibe.

In one embodiment of the invention, the compounds of the formula I, II and III are administered in combination with an antioxidant, such as, for example, OPC-14117.

In one embodiment of the invention, the compounds of the formula I, II and III are administered in combination with a lipoprotein lipase inhibitor, such as, for example, NO-1886.

In one embodiment of the invention, the compounds of the formula I, II and III are administered in combination with an ATP-citrate lyase inhibitor, such as, for example, SB-204990.

In one embodiment of the invention, the compounds of the formula I, II and III are administered in combination with a squalene synthetase inhibitor, such as, for example, BMS-188494.

In one embodiment of the invention, the compounds of the formula I, II and III are administered in combination with a lipoprotein (a) antagonist, such as, for example, CI-1027 or nicotinic acid.

In one embodiment of the invention, the compounds of the formula I, II and III are administered in combination with a lipase inhibitor, such as, for example, orlistat.

In one embodiment of the invention, the compounds of the formula I, II and III are administered in combination with insulin.

In one embodiment, the compounds of the formula I, II and III are administered in combination with a sulfonylurea such as, for example, tolbutamide, glibenclamide, glipizide or glimepiride.

In one embodiment, the compounds of the formula I, II and III are administered in combination with a biguanide, such as, for example, metformin.

In one further embodiment, the compounds of the formula I, II and III are administered in combination with a meglitinide, such as, for example, repaglinide.

In one embodiment, the compounds of the formula I, II and III are administered in combination with a thiazolidinedione, such as, for example, troglitazone, ciglitazone, pioglitazone, rosiglitazone or the compounds disclosed in WO 97/41097 of Dr. Reddy's Research Foundation, in particular 5-[[4-[(3,4-dihydro-3-methyl-4-oxo-2-quinazolinylmethoxy]phenyl]methyl]-2,4-thiazolidinedione.

In one embodiment, the compounds of the formula I, II and III are administered in combination with an α-glucosidase inhibitor, such as, for example, miglitol or acarbose.

In one embodiment, the compounds of the formula I, II and III are administered in combination with an active ingredient which acts on the ATP-dependent potassium channel of the beta cells, such as, for example, tolbutamide, glibenclamide, glipizide, glimepiride or repaglinide.

In one embodiment, the compounds of the formula I, II and III are administered in combination with more than one of the aforementioned compounds, e.g. in combination with a sulfonylurea and metformin, with a sulfonylurea and acarbose, repaglinide and metformin, insulin and a sulfonylurea, insulin and metformin, insulin and troglitazone, insulin and lovastatin, etc.

In a further embodiment, the compounds of the formula I, II and III are administered in combination with CART modulators (see “Cocaine-amphetamine-regulated transcript influences energy metabolism, anxiety and gastric emptying in mice” Asakawa, A, et al., M.: Hormone and Metabolic Research (2001), 33(9), 554-558), NPY antagonists, e.g. naphthalene-1-sulfonic acid {4-[(4-aminoquinazolin-2-ylamino)methyl]-cyclohexyl-methyl}amide; hydrochloride (CGP 71683A)), MC4 agonists (e.g. 1-amino-1,2,3,4-tetrahydronaphthalene-2-carboxylic acid [2-(3a-benzyl-2-methyl-3-oxo-2,3,3a,4,6,7-hexahydropyrazolo[4,3-c]pyridin-5-yl)-1-(4-chlorophenyl)-2-oxoethyl]-amide; (WO 01/91752)), orexin antagonists (e.g. 1-(2-methylbenzoxazol-6-yl)-3-[1,5]naphthyridin-4-ylurea; hydrochloride (SB-334867-A)), H3 agonists (3-cyclohexyl-1-(4,4-dimethyl-1,4,6,7-tetrahydroimidazo[4,5-c]pyridin-5-yl)propan-1-one oxalic acid salt (WO 00/63208)); TNF agonists, CRF antagonists (e.g. [2-methyl-9-(2,4,6-trimethylphenyl)-9H-1,3,9-triazafluoren-4-yl]dipropylamine (WO 00/66585)), CRF BP antagonists (e.g. urocortin), urocortin agonists, 133 agonists (e.g. 1-(4-chloro-3-methanesulfonylmethylphenyl)-2-[2-(2,3-dimethyl-1H-indol-6-yloxy)ethylamino]-ethanol; hydrochloride (WO 01/83451)), MSH (melanocyte-stimulating hormone) agonists, CCK-A agonists (e.g. {2-[4-(4-chloro-2,5-dimethoxyphenyl)-5-(2-cyclohexyl-ethyl)thiazol-2-ylcarbamoyl]-5,7-dimethylindol-1-yl}acetic acid trifluoroacetic acid salt (WO 99/15525)), serotonin reuptake inhibitors (e.g. dexfenfluramine), mixed sertoninergic and noradrenergic compounds (e.g. WO 00/71549), 5HT agonists, e.g. 1-(3-ethylbenzofuran-7-yl)piperazine oxalic acid salt (WO 01/09111), bombesin agonists, galanin antagonists, growth hormone (e.g. human growth hormone), growth hormone-releasing compounds (6-benzyloxy-1-(2-diisopropylaminoethylcarbamoyl)-3,4-dihydro-1H-isoquinoline-2-carboxylic acid tert-butyl ester (WO 01/85695)), TRH agonists (see, for example, EP 0 462 884), uncoupling protein 2 or 3 modulators, leptin agonists (see, for example, Lee, Daniel W.; Leinung, Matthew C.; Rozhayskaya-Arena, Marina; Grasso, Patricia. Leptin agonists as a potential approach to the treatment of obesity. Drugs of the Future (2001), 26(9), 873-881), DA agonists (bromocriptine, Doprexin), lipase/amylase inhibitors (e.g. WO 00/40569), PPAR modulators (e.g. WO 00/78312), RXR modulators or TR-13 agonists.

In one embodiment of the invention, the other active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In one embodiment, the other active ingredient is dexamphatamine or amphetamine.

In one embodiment, the other active ingredient is fenfluramine or dexfenfluramine.

In another embodiment, the other active ingredient is sibutramine.

In one embodiment, the other active ingredient is orlistat.

In one embodiment, the other active ingredient is mazindol or phentermine.

In one embodiment, the compounds of the formula I, II and III are administered in combination with bulking agents, preferably insoluble bulking agents (see, for example, carob/Caromax® (Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Höchst, 65926 Frankfurt/Main)). Combination with Caromax® is possible in one preparation or by separate administration of compounds of the formula I, II and III and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is regarded as falling within the protection conferred by the present invention.

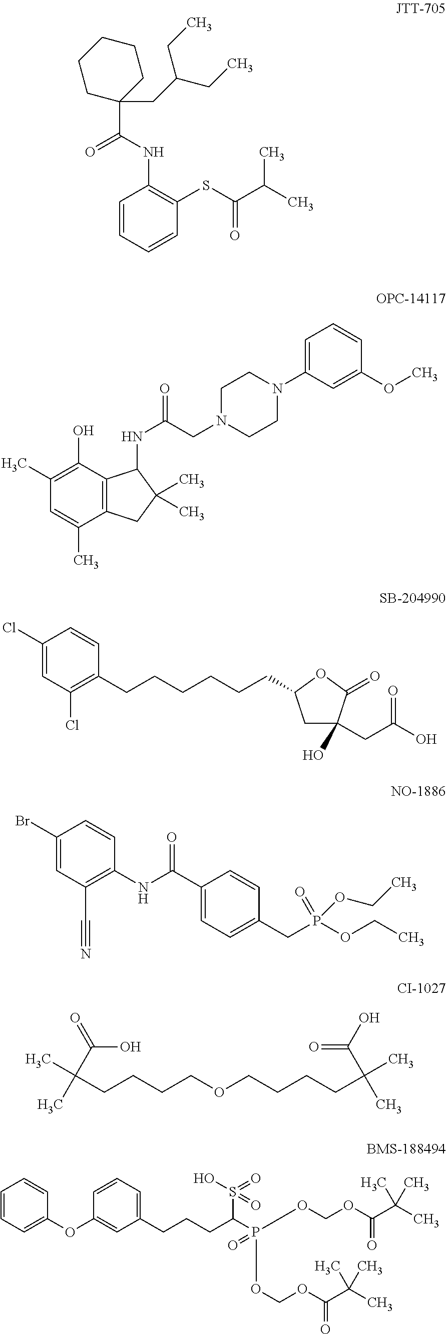

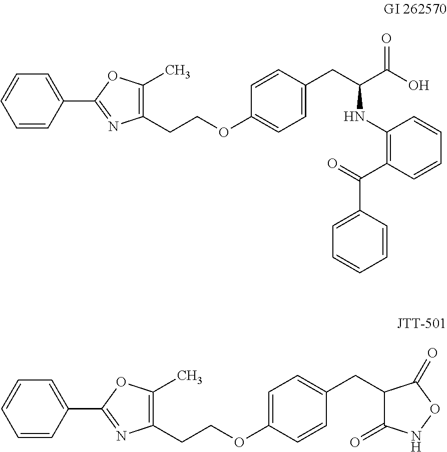

The activity of the compounds was tested as follows:

Preparation of Brush Border Membrane Vesicles from the Small Intestine of Rabbits, Rats and Pigs

Preparation of brush border membrane vesicles from the intestinal cells of the small intestine was carried out by the so-called Mg²⁺ precipitation method. The mucosa of the small intestine was scraped off and suspended in 60 ml of ice-cold Tris/HCl buffer (pH 7.1)/300 mM mannitol, 5 mM EGTA. Dilution to 300 ml with ice-cold distilled water was followed by homogenization with an Ultraturrax (18 shaft, IKA Werk Staufen, FRG) at 75% of the max. power for 2×1 minute, while cooling in ice. After addition of 3 ml of 1M MgCl₂ solution (final concentration 10 mM), the mixture is left to stand at 0° C. for exactly 15 minutes. Addition of Mg²⁺ causes the cell membranes to aggregate and precipitate with the exception of the brush border membranes. After centrifugation at 3 000×g (5000 rpm, SS-34 rotor) for 15 minutes, the precipitate is discarded and the supernatant, which contains the brush border membranes, is centrifuged at 26 700×g (15 000 rpm, SS-34 rotor) for 30 minutes. The supernatant is discarded, and the precipitate is rehomogenized in 60 ml of 12 mM Tris/HCl buffer (pH 7.1)/60 mM mannitol, 5 mM EGTA using a Potter Elvejhem homogenizer (Braun, Melsungen, 900 rpm, 10 strokes). Addition of 0.1 ml of 1M MgCl₂ solution and incubation at 0° C. for 15 minutes is followed by centrifugation again at 3000×g for 15 minutes. The supernatant is then centrifuged again at 46 000×g (20 000 rpm, SS-34 rotor) for 30 minutes. The precipitate is taken up in 30 ml of 20 mM Tris/Hepes buffer (pH 7.4)/280 mM mannitol and homogeneously resuspended by 20 strokes in a Potter Elveihem homogenizer at 1000 rpm. After centrifugation at 48 000×g (20 000 rpm, SS-34 rotor) for 30 minutes, the precipitate was taken up in 0.5 to 2 ml of Tris/Hepes buffer (pH 7.4)/280 mM mannitol (final concentration 20 mg/ml) and resuspended using a tuberculin syringe with a 27 gauge needle.

The vesicles were either used directly after preparation for labeling or transport studies or were stored at −196° C. in 4 mg portions in liquid nitrogen.

To prepare brush border membrane vesicles from rat small intestine, 6 to 10 male Wistar rats (bred at Kastengrund, Aventis Pharma) were sacrificed by cervical dislocation, and the small intestines were removed and rinsed with cold isotonic saline. The intestines were cut up and the mucosa was scraped off. The processing to isolate brush border membranes took place as described above. To remove cytoskeletal fractions, the brush border membrane vesicles from rat small intestine were treated with KSCN as chaotropic ion.

To prepare brush border membranes from rabbit small intestine, rabbits were sacrificed by intravenous injection of 0.5 ml of an aqueous solution of 2.5 mg of tetracaine HCl, 100 mg of m-butramide and 25 mg of mebezonium iodide. The small intestines were removed, rinsed with ice-cold physiological saline and stored frozen in plastic bags under nitrogen at −80° C. and 4 to 12 weeks. For preparation of the membrane vesicles, the frozen intestines were thawed at 30° C. in a water bath and then the mucosa was scraped off. Processing to give membrane vesicles took place as described above.

To prepare brush border membrane vesicles from pig intestine, jejunum segments from a freshly slaughtered pig were rinsed with ice-cold isotonic saline and frozen in plastic bags under nitrogen at −80° C. Preparation of the membrane vesicles took place as described above.

Measurement of the Glucose Uptake by Brush Border Membrane Vesicles

The uptake of [¹⁴C]-labeled glucose into brush border membrane vesicles was measured by the membrane filtration method. 10 μl of the brush border membrane vesicle suspension in 10 mM Tris/Hepes buffer (pH 7.4)/300 mM mannitol were added at 20° C. to 90 μl of a solution of 10 pM [¹⁴C]D glucose and the appropriate concentrations of the relevant inhibitors (5-200 μM) in 10 mM Tris/Hepes buffer (pH 7.4)/100 mM NaCl/100 mM.

After incubation for 15 seconds, the transport process was stopped by adding 1 ml of ice-cold stop solution (10 mM Tris/Hepes buffer (pH 7.4)/150 mM KCl) and the vesicle suspension was immediately filtered with suction through a cellulose nitrate membrane filter (0.45 μm, 25 mm diameter, Schleicher & Schüll) under a vacuum of from 25 to 35 mbar. The filter was washed with 5 ml of ice-cold stop solution. Each measurement was carried out as duplicate or triplicate determination.

To measure the uptake of radiolabeled substrates, the membrane filter was dissolved in 4 ml of an appropriate scintillator (Quickszint 361, Zinsser Analytik GmbH, Frankfurt am Main), and the radioactivity was determined by liquid scintillation measurement.

The measured values were obtained as dpm (decompositions per minute) after calibration of the instrument using standard samples and after correction for any chemiluminescence present.

The active ingredients are compared for activity on the basis of IC₅₀ data obtained in the transport assay on rabbit small intestine brush border membrane vesicles for selected substances. (The absolute values may be species- and experiment-dependent).

A further method for testing the activity of the compounds is the inhibition of the transport activity of the human sodium-dependent glucose transporter 1 (SGLT1, SLC5A1) in vitro:

1. Cloning of an Expression Vector for Human SGLT1

The cDNA for human SGLT1 was introduced into the pcDNA4/TO vector (Invitrogen) by standard methods of molecular biology as described in Sambrook et al. (Sambrook et al., Molecular Cloning, A Laboratory Manual, 2nd Edition). The subsequent sequencing of the insert revealed complete identity with bases 11 to 2005 of the base sequence for human SGLT1 which was described by Hediger et al. (Hediger et al., Proc. Natl. Acad. Sci. USA 1989, 86, 5748-5752.) and deposited in the GenBank sequence database (GenBank Accession Number: M24847). Bases 11 to 2005 correspond to the complete coding region of human SGLT1.

2. Preparation of a Recombinant Cell Line with Inducible Expression of Human SGLT1

The expression vector for human SGLT1 was introduced into CHO-TRex cells (Invitrogen) by means of FuGene6 lipofection (Roche). To select single cell clones, 600 μg/ml Zeocin (Invitrogen) was added to the cell culture medium (Nutrient Mixture F-12 (Ham), (Invitrogen) supplemented with 10% fetal calf serum (BD Biosciences), 10 μg/ml blasticidin S(CN Biosciences), 100 units/ml penicillin, 100 units/ml streptomycin). The functionality of the single cell clones resulting from the selection was tested via their uptake activity for radiolabeled methyl α-D-glucopyranoside. The cell clone with the greatest uptake activity for methyl α-D-glucopyranoside, referred to as CHO-TRex-hSGLT1 hereinafter, was selected for further experiment, and cultivation was continued in the presence of 600 μg/ml of zeocin.

3. Measurement of the Inhibitory Effect of Test Substances on the Uptake of Methyl-D-qlucopyranoside (-MDG)

CHO-TRex-hSGLT1 cells were seeded in a concentration of 50 000 cells per well in Cytostar-T scintillating 96-well plates (Amersham Biosciences) in cell culture medium and cultivated for 24 h. Expression of the recombinant human SGLT1 was induced by adding 1 μg/ml tetracyclin for a further 24 h. For α-MDG uptake experiments, the cells were washed with PBS and then starved (PBS supplemented with 10% fetal calf serum) at 37° C. for one hour. After a further washing step with transport assay buffer (140 mM sodium chloride, 2 mM potassium chloride, 1 mM magnesium chloride, 1 mM calcium chloride, 10 mM HEPES/Tris, pH 7.5), the cells were incubated either in the absence or presence of test substances varying in concentration at room temperature for 15 min. The test substances were diluted appropriately in transport assay buffer (40 μl/well) starting from a 10 mM stock solution in dimethyl sulfoxide. The assay was then started by adding 10 μl of a mixture of radiolabeled methyl α-D-[U-¹⁴C]glucopyranoside (Amersham) and unlabeled methyl α-D-glucopyranoside (Acros).

The final concentration of methyl α-D-glucopyranoside in the assay was 50 μM. After an incubation time of 30 min at room temperature, the reaction was stopped by adding 50 μl/well of 10 mM methyl α-D-glucopyranoside in transport assay buffer (4° C.), and the radioactivity uptake by the cells was determined in a MicroBeta Scintillation Microplate Reader (Wallac). The half-maximum inhibitory effect of the test substances (IC50) was determined in the following way:
1. Establishment of the value for 0% inhibition. This is the measurement in the absence of substance, measured in sodium-containing transport assay buffer.
2. Establishment of the value for 100% inhibition. This is the measurement in the absence of substance, measured in sodium-free transport assay buffer (140 mM choline chloride, 2 mM potassium chloride, 1 mM magnesium chloride, 1 mM calcium chloride, 10 mM HEPES/Tris, pH7.5).
3. Calculation of the percentage inhibitions for the measurements carried out in the presence of various concentrations of test substance. It was then possible to ascertain therefrom the concentration of test substance which reduced the uptake of methyl α-D-glucopyranoside by 50% (IC50).

Inhibition of the Transport Activity of the Human Sodium-Dependent Glucose Transporter 2 (SGLT2, SLC5A2) In Vitro

1. Cloning of an Expression Vector for Human SGLT2

The cDNA for human SGLT2 was introduced into the pcDNA4/TO vector (Invitrogen) by means of standard methods of molecular biology as described in Sambrook et al. (Molecular Cloning, A Laboratory Manual, Second Edition). The subsequent sequencing of the insert showed complete identity with bases 21 to 2039 of the base sequence for human SGLT2 which was described by Wells et al. and is deposited in the GenBank sequence database (GenBank Accession Number: M95549). Bases 21 to 2039 correspond to the complete coding region of human SGLT2.

2. Production of a Recombinant Cell Line with Inducible Expression of Human SGLT2

The expression vector for human SGLT2 was introduced into CHO-TREx cells (Invitrogen) by means of FuGene6 lipofection (Roche). To select single cell clones, 600 μg/ml of Zeocin (Invitrogen) was added to the cell culture medium (nutrient mixture F-12 (Ham), (Invitrogen) supplemented with 10% fetal calf serum (FBS Gold, PAA), 10 μg/ml Blasticidin S(CN Biosciences), 100 units/ml penicillin, 100 units/ml streptomycin). The functionality of the single cell clones resulting from the selection was tested via their uptake activity for radiolabeled methyl- -D-glucopyranoside. That cell clone with the highest uptake activity for methyl- -D-glucopyranoside, referred to hereinafter as CHO-TRex-hSGLT2, was selected for the further experiments and cultured further in the presence of 600 μg/ml Zeocin.

3. Measurement of the Inhibiting Action of Test Substances on the Uptake of Methyl- -D-glucopyranoside (-MDG)

CHO-TRex-hSGLT2 cells were seeded in cell culture medium in a concentration of 50 000 cells per well in Cytostar-T scintillating 96-well plates (Amersham Biosciences) and cultivated for 24 h. The expression of the recombinant human SGLT2 was induced by adding 1 μg/ml tetrazykline for a further 24 h. For -MDG uptake experiments, the cells were washed with PBS and then starved at 37° C. in starvation medium (PBS supplemented with 10% fetal calf serum) for 1 hour. After a further washing step with transport assay buffer (140 mM sodium chloride, 2 mm potassium chloride, 1 mm magnesium chloride, 1 mm calcium chloride, 10 mm HEPES/Tris, pH 7.5), the cells were incubated at room temperature either in the absence or presence of test substances of different concentration for 15 min. The test substances were diluted correspondingly in transport assay buffer proceeding from a 10 mm stock solution in dimethyl sulfoxide (40 μl/well). The assay was subsequently started by adding 10 μl/well of a mixture of radiolabeled methyl- -D-[U-¹⁴C]glucopyranoside (Amersham) and unlabeled methyl- -D-glucopyranoside (Acros). The final concentration of methyl-1-D-glucopyranoside in the assay was 50 μM. After an incubation time of 120 min at 37° C., the reaction was stopped by adding 50 μl/well of 10 mM methyl- -D-glucopyranoside in transport assay buffer (4° C.), and the radioactivity taken up into the cells was determined in a MicroBeta Scintillation Microplate Reader (Wallac). The half-maximum inhibiting action of the test substances (IC50 value) was determined as follows:

4. Determination of the value for 0% inhibition. This is the measurement in the absence of substance, measured in sodium-containing transport assay buffer.

5. Determination of the value for 100% inhibition. This is the measurement in the absence of substance, measured in sodium-free transport assay buffer (140 mM choline chloride, 2 mM potassium chloride, 1 mM magnesium chloride, 1 mM calcium chloride, 10 mM HEPES/Tris, pH7.5).

6. Calculation of the percentage inhibition values of those measurements which were carried out in the presence of different concentrations of test substance. From this, it was then possible to determine that concentration of the test substance which reduces the uptake of the methyl- -D-glucopyranoside by 50% (IC50 value).

Literature:

Wells et al. (1992) Am. J. Physiol. Vol. 263: F459-F465

IC50 values of test substances (μM)

[in vitro testing of the uptake of methyl-D-glucopyranoside]

Formula I

| Example No. | IC₅₀ [M] SGLT 1 |
|---|---|
| 3 | 0.043 |
| 6 | 0.133 |
| 9 | 0.081 |
| 12 | 0.139 |
| 15 | 0.170 |
| 18 | 0.080 |
| 21 | 0.047 |
| 22 | 0.144 |
| 24 | 0.208 |
| 31 | 0.252 |
| 33 | 0.070 |
| 36 | 0.043 |

Formula II

| | IC₅₀ [μM] |
|---|---|
| | SGLT Brush |
| | Border |
| Example No | Membrane |
| Phlorizin | 16 |
| 1 | 4 |
| 2 | 0.4 |
| 3 | 0.3 |

Formula III

| | IC₅₀ [μM] |
|---|---|
| | SGLT Brush |
| | Border |
| Example No | Membrane |
| Phlorizin | 16 |
| 1 | 0.5 |
| 2 | 0.7 |
| 4 | 1.5 |
| 5 | 0.4 |
| 7 | 0.9 |

Following invitro IC₅₀ values of were obtained by using recombinant SGLT1 and SGLT2 targets as described above

| | IC₅₀ [μM] | IC₅₀ [μM] |
|---|---|---|
| Example | SGLT 1 | SGLT 2 |
| 5 of formula II | 0.029 | 0.088 |
| 1 of formula III | 0.770 | 0.034 |
| 20 of formula III | 0.320 | 0.029 |
| 21 of formula I | 0.027 | 0.318 |

The preparation of various examples is described in detail below, and the other compounds of the formula I, II and III were obtained analogously:

Animal Study—Study Design

The compound of example 5 of formula II was tested in female Sprague-Dawley mature and young adult rats following daily oral gavage administration for a minimum of 28 days to investigate specific endpoints of bone quality.

Test Compound:

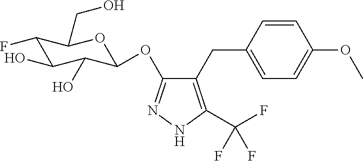

The study design is detailed in the Table below:

Study Design

| | | Number of animals | |
|---|---|---|---|
| | | Mature Female | Young Female |
| Group Number/ | Dose Level | Adult | Adult |
| Identification | (mg/kg/day) | 24 Weeks old^{a} | 6-7 Weeks old^{a} |
| 1/Vehicle Control | 0 | 10 | 10 |
| 2/Test compound | 10 | 15 | 15 |
| 3/Test compound | 50 | 15 | 15 |

^{a}Approximate age at the initiation of treatment

The following were evaluated: clinical signs (at least weekly), body weight (weekly), food consumption (weekly), hormones (Days 13 and 27), serum and urinary biochemical markers of bone turnover (Days 13 and 27), serum chemistry (at necropsy), bone densitometry by dual energy X-ray absorptiometry (DXA) and peripheral quantitative computed tomography (pQCT) (Week 2 and 4, as well as ex vivo), macroscopic observations at necropsy, organ weights, histopathology, biomechanical strength testing and femoral length measurements.

Methods

1 Bone Mineral Density Measurement

Bone densitometry scans and assessments were performed by trained personnel who were blinded to the animals treatment using dual energy X-ray absorptiometry (DXA) and peripheral quantitative computed tomography (pQCT). All animals were scanned using the same densitometer on each occasion. DXA and pQCT scans were performed on the same day in order to reduce any stress to the animal. Animals were anesthetized using isoflurane prior to and during the DXA and pQCT scans. An ophthalmic lubricating ointment was administered to each eye following anesthesia induction and reapplied as necessary.

1.1 In Vivo Dual Energy X-Ray Adsorptiometry (DXA)

Bone mineral density measurements was performed using a Discovery A Hologic densitometer. All scan reanalysis data were retained and reanalysis documented. DXA scans were used to measure bone mineral density (BMD) and bone mineral content (BMC) and area of the whole body, lumbar spine (L1-L4) and right femur (whole with regions of interest at the proximal, mid and distal femur).

Single scans were acquired once prior to treatment start, at the end of Week 2 and again at the end of the treatment period from each study animal. Animals were positioned according to PCS-MTL Standard Operating Procedures. Additional scans were acquired for verification at the request of the study director. The scan modes and analyses are listed in Table 1 below:

TEXT TABLE 1
In Vivo DXA Scan Modes and Analyses

| Scan Site | Scan Type | Scan Mode | Analysis Mode |
|---|---|---|---|
| Whole Body | Small animal/Rat | Array | Small animal/Rat |
| | Whole Body | | Whole Body |
| Lumbar spine | Small animal | High Definition | Subregion High |
| | Regional | | Resolution Analysis |
| | High-Resolution | | |
| Femur | Small animal | High Definition | Subregion High |
| | Regional | | Resolution Analysis |
| | High-Resolution | | |

1.2 Peripheral Quantitative Computed Tomography (pQCT)

Peripheral QCT was used to measure bone mineral content (BMC), bone mineral density (BMD), and geometric parameters for all animals.

Peripheral QCT scans were obtained at the right proximal tibia for all animals using an XCT Research SA or SA+ bone scanner with software version 5.50D. Scans were acquired at the proximal tibia metaphysis and diaphysis (single slice at each site).

Additional slices were obtained as considered appropriate to ensure the optimal scans and data acquisition. In this case, only the best scan was considered. The exact position of the scan slices was documented in the raw data (Table 4). For follow-up scans, positioning and placement of CT scan lines were verified and compared with the scout scan obtained during the initial scanning occasion.

For the metaphysis site, the most appropriate analysis modes to represent the scans was determined, documented and reported in the Table 2. Scans obtained at the diaphysis site were analyzed using Cortmode 2 (Table 2). Scanning parameters reported are summarized in the Table 3. All scan analysis data was retained and reanalysis documented. All analyses were performed using the LOOP option. All other data generated as a result of the LOOP option was retained with the raw data but not reported.

TEXT TABLE 2
In Vivo pQCT Scan Settings and Analysis Using
Software Version 5.50D

| | Right Tibia |
|---|---|
| Approximate Measurement Diameter | 30 |
| (mm) | |
| Voxel Size: (mm) | 0.15 |
| CT scan speed (mm/sec) | 4.5 |
| SV scan speed (mm/sec) | 30 |
| Number of SV lines | 99 |
| Dist. between SV lines (mm) | 0.5 |
| # Blocks | 1 |
| Dist between CT lines (mm) | 0.2 |
| Metaphysis | Acquired at 15% of the total |
| Slice Placement | bone length distal to the reference |
| | line set at the tibial proximal end |
| # of CT slices | 1 |
| ContMode | 2 |
| PeelMode | 2 threshold 0.850 1/cm |
| Diaphysis | |
| Slice Placement | Acquired at 50% of the total |
| | bone length |
| # of CT slices | 1 |
| Cortmode | 2, threshold 0.930 1/cm |

TEXT TABLE 3
pQCT Scanning parameters reported for the right tibia

| Site | Region | Parameter | Abbreviation | Unit |
|---|---|---|---|---|
| Metaphysis | Total slice | Area | None | mm² |
| | | Bone mineral | BMC | mg/mm |
| | | content | | |
| | | Bone mineral | BMD | mg/cm³ |
| | | density | | |
| | Trabecular | Area | None | mm² |
| | subregion | Bone mineral | BMC | mg/mm |
| | | content | | |
| | | Bone mineral | BMD | mg/cm³ |
| | | density | | |
| | Cortical/ | Area | None | mm² |
| | Subcortical | Bone mineral | BMC | mg/mm |
| | | content | | |
| | subregion | Bone mineral | BMD | mg/cm³ |
| | | density | | |
| Diaphysis | | Periosteal | PERI_C | mm |
| | | circumference | | |
| | | Endosteal | ENDO_C | mm |
| | | circumference | | |
| | Total slice | Area | None | mm² |
| | Cortical | Area | None | mm² |
| | | Bone mineral | BMC | mg/mm |
| | | content | | |
| | | Bone mineral | BMD | mg/cm³ |
| | | density | | |
| | | Thickness | THICK_C | mm |

Results of bone mineral density measurements are shown in following figures and tables:

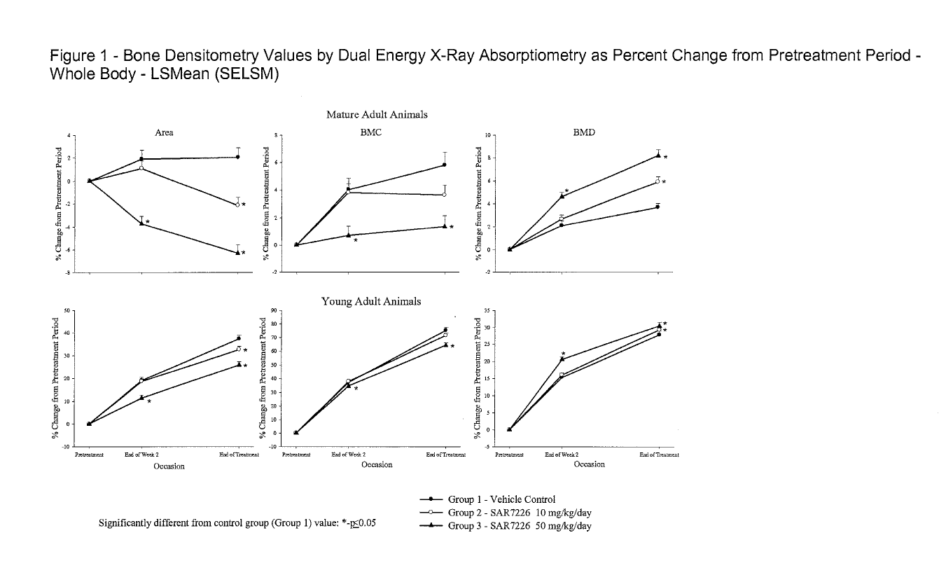

FIG. 1—Bone Densitometry Values by Dual Energy X-Ray Absorptiometry as Percent Change from Pretreatment Period-Whole Body -LSMean (SELSM)

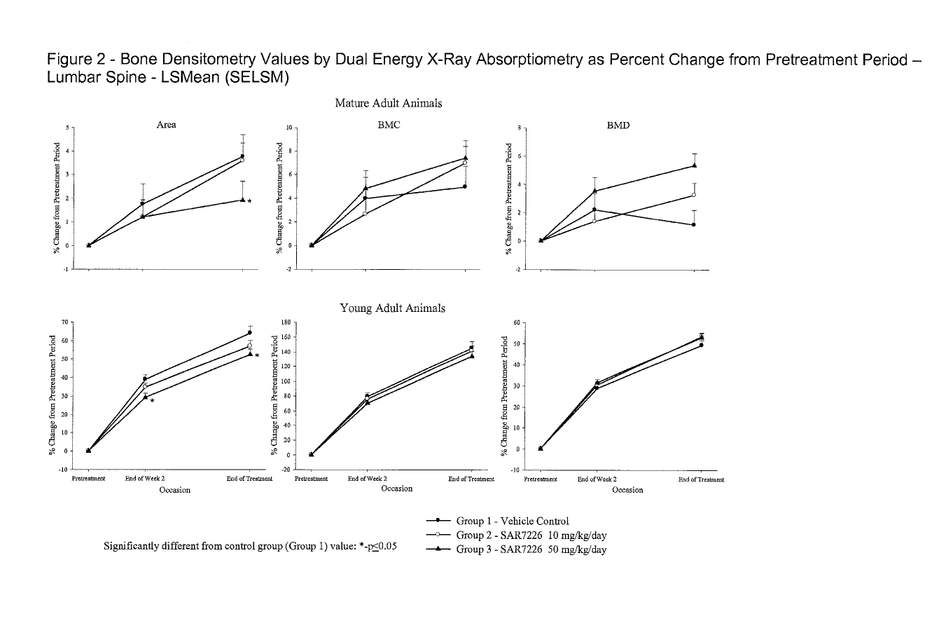

FIG. 2—Bone Densitometry Values by Dual Energy X-Ray Absorptiometry as Percent Change from Pretreatment Period-Lumbar Spine-LSMean (SELSM)

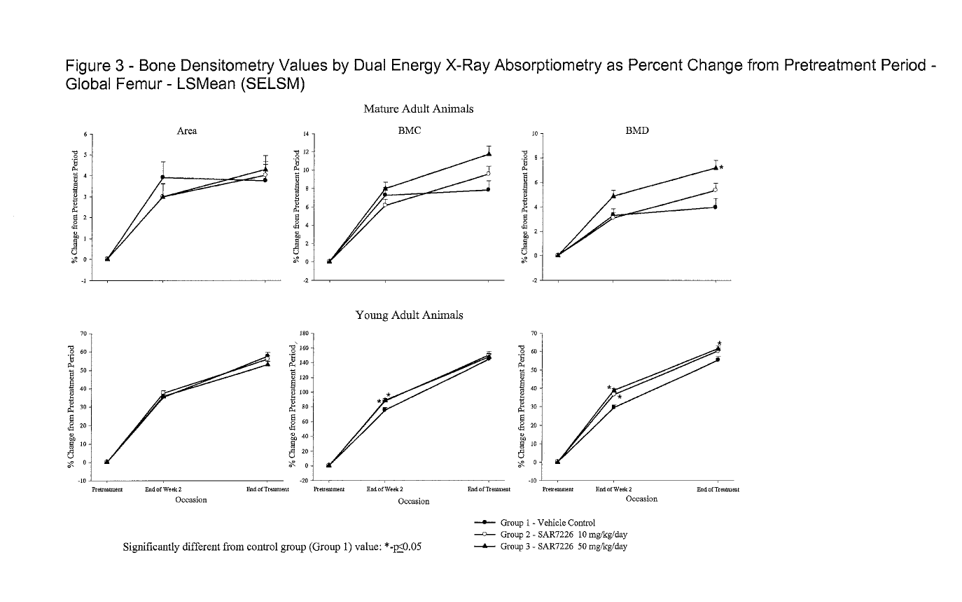

FIG. 3—Bone Densitometry Values by Dual Energy X-Ray Absorptiometry as Percent Change from Pretreatment Period-Global Femur-LSMean (SELSM)

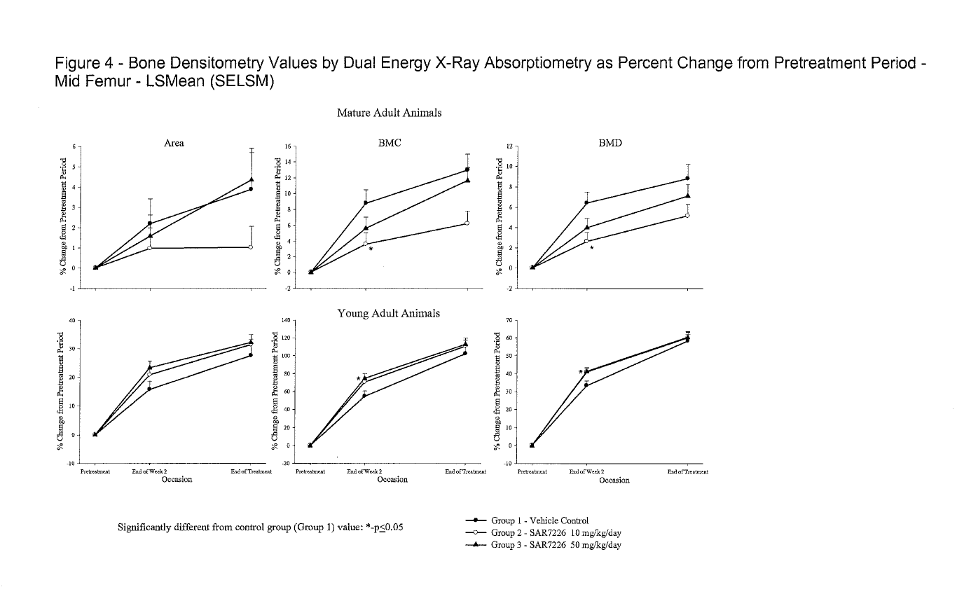

FIG. 4—Bone Densitometry Values by Dual Energy X-Ray Absorptiometry as Percent Change from Pretreatment Period-Mid Femur-LSMean (SELSM)

TEXT TABLE 1a
Bone Densitometry by DXA - Summary Data Percent Difference
from Vehicle - Femur - Ex Vivo - Mature Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| DXA | Global | cm² | Mean | 1.87 | 1.91 | 1.9 | 1.85 | −1.2 |
| | Area | | SD | 0.10 | 0.10 | | 0.08 | |
| | Global | g | Mean | 0.49 | 0.52 | 6.0 | 0.51 | 3.7 |
| | BMC | | SD | 0.04 | 0.05 | | 0.03 | |
| | Global | g/cm² | Mean | 0.26 | 0.27 | 4.0 | 0.27 | 5.0 |
| | BMD | | SD | 0.01 | 0.01 | | 0.01 | |
| | Prox- | cm² | Mean | 0.61 | 0.63 | 2.4 | 0.61 | 0.1 |
| | imal | | SD | 0.03 | 0.03 | | 0.02 | |
| | Area | | | | | | | |
| | Prox- | g | Mean | 0.17 | 0.18 | 6.1 | 0.17 | 3.0 |
| | imal | | SD | 0.01 | 0.01 | | 0.01 | |
| | BMC | | | | | | | |
| | Prox- | g/cm² | Mean | 0.27 | 0.28 | 3.7 | 0.28 | 2.9 |
| | imal | | SD | 0.01 | 0.01 | | 0.01 | |
| | BMD | | | | | | | |
| | Mid | cm² | Mean | 0.44 | 0.45 | 3.9 | 0.43 | −1.0 |
| | Area | | SD | 0.02 | 0.03 | | 0.02 | |
| | Mid | g | Mean | 0.10 | 0.11 | 6.4 | 0.11 | 1.2 |
| | BMC | | SD | 0.01 | 0.01 | | 0.01 | |
| | Mid | g/cm² | Mean | 0.24 | 0.24 | 2.4 | 0.24 | 2.3 |
| | BMD | | SD | 0.01 | 0.01 | | 0.01 | |
| | Distal | cm² | Mean | 0.41 | 0.41 | 1.7 | 0.41 | −0.1 |
| | Area | | SD | 0.02 | 0.01 | | 0.02 | |
| | Distal | g | Mean | 0.13 | 0.14 | 6.2 | 0.14 | 8.3 |
| | BMC | | SD | 0.01 | 0.01 | | 0.01 | |
| | Distal | g/cm² | Mean | 0.33 | 0.34 | 4.4 | 0.35 | 8.4 |
| | BMD | | SD | 0.01 | 0.02 | | 0.01 | |

Values in bold are significantly different from Group 1 values (Statistical analysis were performed on the least square mean)
% = percent difference from Vehicle Control

TEXT TABLE 2a
Bone Densitometry by DXA - Summary Data Percent Difference
from Vehicle - Femur - Ex Vivo - Young Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| DXA | Global | cm² | Mean | 1.49 | 1.45 | −2.5 | 1.42 | −4.7 |
| | Area | | SD | 0.08 | 0.09 | | 0.08 | |
| | Global | g | Mean | 0.30 | 0.30 | 0.5 | 0.30 | 0.0 |
| | BMC | | SD | 0.03 | 0.03 | | 0.03 | |
| | Global | g/cm² | Mean | 0.20 | 0.21 | 3.2 | 0.21 | 4.9 |
| | BMD | | SD | 0.01 | 0.01 | | 0.01 | |
| | Prox- | cm² | Mean | 0.52 | 0.50 | −2.6 | 0.49 | −4.9 |
| | imal | | SD | 0.02 | 0.03 | | 0.03 | |
| | Area | | | | | | | |
| | Prox- | g | Mean | 0.11 | 0.11 | −0.8 | 0.11 | −4.0 |
| | imal | | SD | 0.01 | 0.01 | | 0.01 | |
| | BMC | | | | | | | |
| | Prox- | g/cm² | Mean | 0.21 | 0.22 | 1.8 | 0.21 | 1.0 |
| | imal | | SD | 0.01 | 0.01 | | 0.01 | |
| | BMD | | | | | | | |
| | Mid | cm² | Mean | 0.39 | 0.39 | −0.9 | 0.39 | −0.8 |
| | Area | | SD | 0.01 | 0.02 | | 0.03 | |
| | Mid | g | Mean | 0.07 | 0.07 | −2.6 | 0.07 | −3.2 |
| | BMC | | SD | 0.00 | 0.01 | | 0.01 | |
| | Mid | g/cm² | Mean | 0.17 | 0.17 | −1.9 | 0.17 | −2.6 |
| | BMD | | SD | 0.01 | 0.01 | | 0.01 | |
| | Distal | cm² | Mean | 0.36 | 0.37 | 1.7 | 0.37 | 0.5 |
| | Area | | SD | 0.02 | 0.02 | | 0.02 | |
| | Distal | g | Mean | 0.09 | 0.10 | 6.4 | 0.10 | 8.4 |
| | BMC | | SD | 0.01 | 0.01 | | 0.01 | |
| | Distal | g/cm² | Mean | 0.25 | 0.26 | 4.7 | 0.27 | 7.9 |
| | BMD | | SD | 0.01 | 0.01 | | 0.02 | |

Values in bold are significantly different from Group 1 values (Statistical analysis were performed on the least square mean)
% = percent difference from Vehicle Control

TEXT TABLE 3a
Bone Densitometry by pQCT - Summary Data Percent Difference
from Vehicle Ex Vivo - Femur Metaphysis - Mature Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| pQCT | Total | mm² | Mean | 15.3 | 16.0 | 4.4 | 14.9 | −2.7 |
| | Area | | SD | 1.8 | 1.8 | | 1.4 | |
| | Total | mg/mm | Mean | 11.7 | 12.6 | 7.8 | 12.3 | 5.1 |
| | BMC | | SD | 1.3 | 1.2 | | 1.1 | |
| | Total | mg/cm³ | Mean | 764.4 | 791.1 | 3.5 | 825.3 | 8.0 |
| | BMD | | SD | 48.5 | 59.0 | | 37.9 | |
| | Trabecular | mg/mm | Mean | 2.2 | 2.5 | 14.7 | 2.4 | 9.7 |
| | BMC | | SD | 0.5 | 0.5 | | 0.5 | |
| | Trabecular | mg/cm³ | Mean | 361.0 | 396.9 | 9.9 | 405.3 | 12.3 |
| | BMD | | SD | 74.8 | 62.3 | | 65.4 | |
| | C/SC | mg/mm | Mean | 9.5 | 10.1 | 6.2 | 9.9 | 4.0 |
| | BMC | | SD | 0.9 | 0.7 | | 0.6 | |
| | C/SC | mg/cm³ | Mean | 1033.4 | 1054.0 | 2.0 | 1105.5 | 7.0 |
| | BMD | | SD | 45.0 | 73.6 | | 46.9 | |

Values in bold are significantly different from Group 1 values (Statistical analysis were performed on the least square mean)
% = percent difference from Vehicle Control
C/SC = Cortical/Subcortical

TEXT TABLE 4a
Bone Densitometry by pQCT - Summary Data Percent Difference
from Vehicle Ex Vivo - Femur Metaphysis - Young Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| pQCT | Total | mm² | Mean | 14.4 | 15.0 | 3.7 | 15.1 | 4.3 |
| | Area | | SD | 1.3 | 1.2 | | 1.7 | |
| | Total | mg/mm | Mean | 8.9 | 10.3 | 16.7 | 11.8 | 33.0 |
| | BMC | | SD | 1.0 | 1.1 | | 1.6 | |
| | Total | mg/cm³ | Mean | 612.9 | 690.2 | 12.6 | 782.0 | 27.6 |
| | BMD | | SD | 39.4 | 44.0 | | 52.1 | |
| | Trabecular | mg/mm | Mean | 1.8 | 2.5 | 41.4 | 3.4 | 92.0 |
| | BMC | | SD | 0.4 | 0.5 | | 0.6 | |
| | Trabecular | mg/cm³ | Mean | 301.1 | 410.7 | 36.4 | 556.0 | 84.7 |
| | BMD | | SD | 65.7 | 64.3 | | 65.7 | |
| | C/SC | mg/mm | Mean | 7.1 | 7.9 | 10.7 | 8.4 | 18.4 |
| | BMC | | SD | 0.7 | 0.7 | | 1.0 | |
| | C/SC | mg/cm³ | Mean | 821.0 | 877.1 | 6.8 | 932.6 | 13.6 |
| | BMD | | SD | 23.9 | 37.2 | | 49.5 | |

Values in bold are significantly different from Group 1 values (Statistical analysis were performed on the least square mean)
% = percent difference from Vehicle Control
C/SC = Cortical/Subcortical

2 Biomechanical Testing

Biomechanical testing was performed using an 858 Mini Bionix Servohydraulic Test System, Model 242.03. All data was collected using TestWorks® version 3.8A for TestStar™ software, version 4.0C.

At necropsy, all specimens were cleaned of excess tissue (not scraped) and individually wrapped in gauze soaked with saline, covered with plastic film and placed in an appropriately labelled plastic bag. Bone samples were retained frozen (ca −20° C.) until subjected to the following biomechanical evaluations:

| Bone | Test |
|---|---|
| Right femur | 3-point bending |
| Lumbar vertebrae (L3, L4) | compression testing |

2.1 Specimen Preparation

Specimens were thawed in the fridge (approximately 4° C.) overnight before preparation or taken directly from the freezer on the trimming day (no impact on testing as preparation performed on different day than testing) as follows:

For 3-point bending, the right femur was cleaned of soft tissue, especially at the diaphysis in the area of the span.

For compression testing, the vertebral body of the vertebrae was isolated by removing the vertebral arch and removing both inter-vertebral discs. The section obtained had two parallel cut surfaces and the trabecular bone exposed.

2.2 3-Point Bending

The right femur was tested to failure in three-point bending to determine the material properties of femoral cortical bone. The marked point on the anterior side served as the upper loading point for each femur and the actuator was set at a rate of 1 mm/sec until failure occurred. Load and displacement data were collected. Peak load was determined from the resulting load versus displacement curve and converted to ultimate stress using the radius, cross-sectional moment of inertia and the span. The work to failure was determined from the area under the curve (AUC) and toughness was calculated. Approximate midspan values for stiffness were recorded (defined as the slope of the linear/elastic region of the load versus displacement curve) and modulus was calculated using the moment of inertia obtained from the pQCT data.

2.3 Vertebral Compression

The L3 and L4 vertebral body were tested in compression to failure. The loading rate was set at 20 mm/min. Load and displacement data were collected and apparent strength and modulus were calculated using the peak load, stiffness, individual vertebral body height and cross sectional area from the pQCT scans. The work to failure was determined from the area under the curve (AUC) and toughness was calculated. Yield load was originally derived using a 0.2% off-set however a 0.1% off-set was considered more appropriate and yield stress was calculated. All data derived from the 0.2% off-set yield load was kept with the study file but not reported.

A summary of biomechanical tests and parameters reported are outlined in Table 4.

TEXT TABLE 4
Biomechanical Parameters Reported

| Bone | Test | Parameter | Unit |
|---|---|---|---|
| Right Femur | 3-Point Bending | Peak load | N |
| | | Ultimate stress | MPa |
| | | Stiffness | N/mm |
| | | Modulus | MPa |
| | | Area under the | N-mm |
| | | curve (AUC) | |
| | | Toughness | MPa |
| Lumbar Vertebrae | Compression | Peak load | N |
| (L3, L4) | | Apparent Strength | MPa |
| | | Yield Load | N |
| | | Yield Stress | MPa |
| | | Stiffness | N/mm |
| | | Modulus | MPa |
| | Caliper | Area under the | N-mm |
| | | curve (AUC) | |
| | | Toughness | MPa |
| | | Height | mm |

3 Ex Vivo Bone Mineral Density Measurements

3.1 Ex Vivo Dual Energy X-Ray Absorptiometry (DXA)

DXA was used to measure bone mineral density (BMD), bone mineral content (BMC) and area. Single DXA scans were obtained for the excised right femur from all animals euthanized at the end of the treatment period (except three animals due to damaged femoral condyles) as well as two found dead animals. These exclusions had no adverse impact on the overall outcome of the study as sufficient specimens were measured in each groups. Scans were performed with a Hologic Discovery A bone densitometer as per PCS-MTL Standard Operating Procedures.

Scan sites to be reported are summarized in Table 5:

TEXT TABLE 5
Ex Vivo DXA scan site and parameters reported

| | Reporting |
|---|---|
| Site | Area, BMC, BMD |
| Right Femur | Global, proximal, mid, distal |

3.2 Ex Vivo Peripheral Quantitative Computed Tomography (pQCT)

Peripheral QCT scans were performed ex vivo using an XCT Research SA or SA+ bone scanner with software version 5.50D. Scans were obtained for the right femur, L3 and L4 lumbar vertebra for all animals euthanized at the end of the treatment period as well as two found dead animals. These two animals were scanned twice due to technical oversight, however only the first scans obtained were reported and the second scans were kept with the study file, this had no impact on the study as both scans were considered equivalent.

For the femurs, the scanned site was at the expected 3-point bending fracture site (diaphysis). This site was analyzed using Cortmode 2 for cortical bone measurements only (Table 5). An additional slice was obtained in the metaphysis. The metaphysis scan was not performed for the femur of three animals due to condyles detachment from the femur during trimming. The exact position of the scan slices was documented in the raw data and presented in Table 5.

The femoral length was measured as part of the ex vivo pQCT scanning procedures and was reported for the right femur of all animals with the exception of 3 animas due to damaged femoral condyles.

For the vertebrae, the scans were obtained at the midpoint. The L3 vertebrae from four animal and L4 vertebrae from four animals were damaged at trimming and L4 vertebrae from one animal was lost during trimming, consequently, no scans were obtained on these vertebrae. Additional slices were obtained as considered appropriate to ensure the optimal scans and data acquisition.

All analyses were performed using the LOOP option of the analysis software. All other data generated as a result of the LOOP option were retained with the raw data but not reported. The most appropriate analysis mode for the femur and the lumbar vertebrae was used and documented in the raw data and the analysis details are presented in Table 6. All scan analysis data was retained and re-analyses documented.

TEXT TABLE 6
Ex vivo pQCT Scan Settings and Analysis Using
Software Version 5.50D

| | Femur | Femur | L3 & L4 |
|---|---|---|---|
| | Metaphysis | Diaphysis | Vertebra |
| Approximate | 30 | 30 | 15 |
| Measurement | | | |
| Diameter (mm) | | | |
| Voxel Size: (mm) | 0.10 | 0.10 | 0.10 |
| CT scan speed | 3.0 | 3.0 | 3.0 |
| (mm/sec) | | | |
| SV scan speed | 30 | N/A | 30 |
| (mm/sec) | | | |
| Number of SV lines | 99 | N/A | 99 |
| Distance between SV | 0.50 | N/A | 0.20 |
| lines (mm) | | | |
| # Blocks | 1 | 1 | 1 |
| Distance between CT | N/A | N/A | N/A |
| lines (mm) | | | |
| Slice Placement | On screen, | Laser set on the | On screen, |
| | reference line | mark on the | reference line |
| | placed at the distal | femoral shaft (at | placed at cranial |
| | end of femur and | expected breaking | end of vertebra |
| | scanning line | point) | and scanning line |
| | placed at 20% of | | placed at 50% of |
| | the total length of | | the total length of |
| | the femur | | the vertebra. |
| # of CT slices | 1 | 1 | 1 |
| | (additional slices | | (additional slices |
| | might be needed) | | might be needed) |
| ContMode | 2 | N/A | 2 |
| PeelMode | 20 at 40% | N/A | 20 at 45% |
| | trabecular area | | trabecular area |
| Cortmode | N/A | 2 threshold | N/A |
| | | 0.930 1/cm | |

N/A = Not Applicable

Ex vivo pQCT scanning parameters reported are summarized in Table 7.

TEXT TABLE 7
Ex vivo pQCT Scanning Parameters Reported for the Vertebrae and
Right Femur.

| | | | |
|---|---|---|---|
| Site | Parameter | Abbreviation | Unit |

L3, L4 vertebral body mid section

| | | | |
|---|---|---|---|
| Total and | Cross sectional | None | mm² |
| Trabecular | area | | |
| | Bone mineral | BMC | mg/mm |
| | content | | |
| | Bone mineral | BMD | mg/cm³ |
| | density | | |

Right femur - Diaphysis - at the expected 3 point breaking point

| | | | |
|---|---|---|---|
| | Periosteal | PERI | mm |
| | circumference | | |
| | Endosteal | ENDO | mm |
| | circumference | | |
| Total and Cortical | Cross sectional | None | mm² |
| | area | | |
| | Bone mineral | BMC | mg/mm |
| | content | | |
| | Bone mineral | BMD | mg/cm³ |
| | density | | |
| Cortical | Cross sectional | CSMI | mm⁴ |
| | moment of inertia in | | |
| | the plane of | | |
| | bending | | |
| | Cortical thickness | THICK | mm |

Right Femur - Metaphysis

| | | | |
|---|---|---|---|
| Total slice | Area | None | mm² |
| | Bone mineral | BMC | mg/mm |
| | content | | |
| | Bone mineral | BMD | mg/cm³ |
| | density | | |
| Trabecular | Area | None | mm² |
| subregion | Bone mineral | BMC | mg/mm |
| | content | | |
| | Bone mineral | BMD | mg/cm³ |
| | density | | |
| Cortical/Subcortical | Area | None | mm² |
| Subregion | Bone mineral | BMC | mg/mm |
| | content | | |
| | Bone mineral | BMD | mg/cm³ |
| | density | | |

4.4 Vertebral Compression

The L3 and L4 vertebral body were tested in compression to failure. The loading rate was set at 20 mm/min. Load and displacement data were collected and apparent strength and modulus were calculated using the peak load, stiffness, individual vertebral body height and cross sectional area from the pQCT scans. The work to failure was determined from the area under the curve (AUC) and toughness was calculated. Yield load was originally derived using a 0.2% off-set however a 0.1% off-set was considered more appropriate and yield stress was calculated. All data derived from the 0.2% off-set yield load was kept with the study file but not reported.

A summary of biomechanical tests and parameters reported are outlined in Table 8.

TEXT TABLE 8
Biomechanical Parameters Reported

| Bone | Test | Parameter | Unit |
|---|---|---|---|
| Right Femur | 3-Point Bending | Peak load | N |
| | | Ultimate stress | MPa |
| | | Stiffness | N/mm |
| | | Modulus | MPa |
| | | Area under the | N-mm |
| | | curve (AUC) | |
| | | Toughness | MPa |
| Lumbar Vertebrae | Compression | Peak load | N |
| (L3, L4) | | Apparent Strength | MPa |
| | | Yield Load | N |
| | | Yield Stress | MPa |
| | | Stiffness | N/mm |
| | | Modulus | MPa |
| | Caliper | Area under the | N-mm |
| | | curve (AUC) | |
| | | Toughness | MPa |
| | | Height | mm |

Results are shown in following tables:

TEXT TABLE 5a
Biomechanics - Summary Data Percent Difference from Vehicle -
Femur 3-point Bending - Ex Vivo - Mature Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| Peak Load | | N | Mean | 136.0 | 149.8 | 10.1 | 142.1 | 4.5 |
| | | | SD | 29.3 | 27.6 | | 26.2 | |
| Ultimate Stress | | MPa | Mean | 189.0 | 200.5 | 6.1 | 195.4 | 3.4 |
| | | | SD | 43.0 | 41.8 | | 35.9 | |
| Stiffness | | N/mm | Mean | 404.9 | 410.2 | 1.3 | 401.4 | −0.9 |
| | | | SD | 63.6 | 58.0 | | 87.3 | |
| Modulus | | MPa | Mean | 6148.9 | 6137.7 | −0.2 | 6222.5 | 1.2 |
| | | | SD | 750.6 | 1727.7 | | 1259.3 | |
| AUC | | N-mm | Mean | 56.5 | 64.1 | 13.4 | 61.4 | 8.7 |
| | | | SD | 31.7 | 21.9 | | 22.4 | |
| Toughness | | MPa | Mean | 7.1 | 7.8 | 10.5 | 7.5 | 6.6 |
| | | | SD | 3.9 | 2.7 | | 2.9 | |
| pQCT | Length | mm | Mean | 37.2 | 37.0 | −0.4 | 37.0 | −0.4 |
| | | | SD | 1.2 | 1.0 | | 0.8 | |
| | Total | mm² | Mean | 9.3 | 9.6 | 3.1 | 9.0 | −3.4 |
| | Area | | SD | 1.1 | 1.2 | | 0.7 | |
| | Total | mg/mm | Mean | 8.8 | 9.1 | 3.0 | 8.8 | 0.0 |
| | BMC | | SD | 0.9 | 0.9 | | 0.5 | |
| | Total | mg/cm³ | Mean | 954.5 | 955.6 | 0.1 | 988.0 | 3.5 |
| | BMD | | SD | 31.4 | 56.6 | | 42.7 | |
| | Cortical | mm² | Mean | 6.3 | 6.5 | 2.3 | 6.4 | 0.2 |
| | Area | | SD | 0.6 | 0.6 | | 0.4 | |
| | Cortical | mg/mm | Mean | 8.3 | 8.5 | 2.7 | 8.4 | 1.2 |
| | BMC | | SD | 0.7 | 0.7 | | 0.5 | |
| | Cortical | mg/cm³ | Mean | 1306.7 | 1312.6 | 0.5 | 1319.4 | 1.0 |
| | BMD | | SD | 24.8 | 16.9 | | 18.7 | |
| | CSMI | mm⁴ | Mean | 4.7 | 5.0 | 5.7 | 4.6 | −3.0 |
| | | | SD | 1.1 | 1.3 | | 0.8 | |
| | Cortical | mm | Mean | 0.75 | 0.76 | 0.6 | 0.78 | 3.5 |
| | Thickness | | SD | 0.03 | 0.04 | | 0.03 | |
| | Periosteal | mm | Mean | 10.7 | 10.8 | 1.0 | 10.4 | −2.1 |
| | Circumference | | SD | 0.6 | 0.6 | | 0.4 | |
| | Endosteal | mm | Mean | 6.0 | 6.2 | 2.3 | 5.7 | −5.6 |
| | Circumference | | SD | 0.6 | 0.7 | | 0.5 | |
| DXA | Mid | cm² | Mean | 0.44 | 0.45 | 3.86 | 0.43 | −0.97 |
| | Area | | SD | 0.02 | 0.03 | | 0.02 | |
| | Mid | g | Mean | 0.10 | 0.11 | 6.45 | 0.11 | 1.25 |
| | BMC | | SD | 0.01 | 0.01 | | 0.01 | |
| | Mid | g/cm² | Mean | 0.24 | 0.24 | 2.35 | 0.24 | 2.26 |
| | BMD | | SD | 0.01 | 0.01 | | 0.01 | |

% = percent difference from Vehicle Control

TEXT TABLE 6a
Biomechanic - Summary Data Percent Difference
from Vehicle - Femur - Ex Vivo - Young Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| Peak Load | | N | Mean | 97.0 | 97.9 | 0.9 | 94.1 | −3.0 |
| | | | SD | 7.3 | 10.2 | | 9.3 | |
| Ultimate Stress | | MPa | Mean | 153.7 | 155.8 | 1.3 | 150.2 | −2.3 |
| | | | SD | 14.7 | 11.4 | | 19.0 | |
| Stiffness | | N/mm | Mean | 278.4 | 277.3 | −0.4 | 283.9 | 2.0 |
| | | | SD | 22.1 | 40.2 | | 35.2 | |
| Modulus | | MPa | Mean | 5046.3 | 5154.1 | 2.1 | 5249.6 | 4.0 |
| | | | SD | 330.9 | 865.6 | | 931.3 | |
| AUC | | N-mm | Mean | 61.2 | 63.7 | 4.1 | 49.0 | −19.9 |
| | | | SD | 7.3 | 19.2 | | 14.5 | |
| Toughness | | MPa | Mean | 8.5 | 8.7 | 2.5 | 6.8 | −19.2 |
| | | | SD | 1.4 | 2.4 | | 2.3 | |
| pQCT | Length | mm | Mean | 33.2 | 31.9 | −3.8 | 31.6 | −4.9 |
| | | | SD | 0.8 | 1.3 | | 0.9 | |
| | Total | mm² | Mean | 8.6 | 8.6 | −0.9 | 8.6 | −0.7 |
| | Area | | SD | 0.5 | 0.6 | | 1.0 | |
| | Total | mg/mm | Mean | 6.2 | 6.2 | 0.2 | 6.2 | 0.0 |
| | BMC | | SD | 0.4 | 0.4 | | 0.7 | |
| | Total | mg/cm³ | Mean | 713.8 | 721.8 | 1.1 | 720.6 | 0.9 |
| | BMD | | SD | 23.3 | 29.3 | | 43.0 | |
| | Cortical | mm² | Mean | 4.7 | 4.7 | −0.4 | 4.7 | −0.6 |
| | Area | | SD | 0.3 | 0.3 | | 0.4 | |
| | Cortical | mg/mm | Mean | 5.6 | 5.6 | −0.8 | 5.6 | −0.7 |
| | BMC | | SD | 0.3 | 0.4 | | 0.6 | |
| | Cortical | mg/cm³ | Mean | 1187.6 | 1182.8 | −0.4 | 1186.1 | −0.1 |
| | BMD | | SD | 11.9 | 14.8 | | 20.5 | |
| | CSMI | mm⁴ | Mean | 3.9 | 3.8 | −1.3 | 3.9 | 0.6 |
| | | | SD | 0.3 | 0.6 | | 0.8 | |
| | Cortical | mm | Mean | 0.54 | 0.54 | 0.15 | 0.54 | −0.01 |
| | Thickness | | SD | 0.03 | 0.02 | | 0.04 | |
| | Periosteal | mm | Mean | 10.3 | 10.2 | −1.2 | 10.2 | −0.3 |
| | Circumference | | SD | 0.3 | 0.5 | | 0.6 | |
| | Endosteal | mm | Mean | 7.0 | 6.9 | −0.7 | 7.0 | −0.7 |
| | Circumference | | SD | 0.3 | 0.4 | | 0.6 | |
| DXA | Mid | cm² | Mean | 0.39 | 0.39 | −0.89 | 0.39 | −0.77 |
| | Area | | SD | 0.01 | 0.02 | | 0.03 | |
| | Mid | g | Mean | 0.07 | 0.07 | −2.61 | 0.07 | −3.16 |
| | BMC | | SD | 0.00 | 0.01 | | 0.01 | |
| | Mid | g/cm² | Mean | 0.17 | 0.17 | −1.87 | 0.17 | −2.62 |
| | BMD | | SD | 0.01 | 0.01 | | 0.01 | |

Values in bold are significantly different from Group 1 values (Statistical analysis were performed on the least square mean)
% = percent difference from Vehicle Control

TEXT TABLE 7a
Biomechanic - Summary Data Percent Difference from
Vehicle L3 + L4 Vertebral Bodies - Mature Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| Peak Load | | N | Mean | 354.1 | 375.5 | 6.0 | 430.6 | 21.6 |
| | | | SD | 58.7 | 59.0 | | 63.5 | |
| Apparent Strength | | MPa | Mean | 46.7 | 48.3 | 3.4 | 53.3 | 14.1 |
| | | | SD | 4.0 | 6.2 | | 5.4 | |
| Yield Load | | N | Mean | 315.6 | 329.3 | 4.3 | 368.0 | 16.6 |
| | | | SD | 51.1 | 56.3 | | 72.8 | |
| Yield Stress | | MPa | Mean | 41.6 | 42.5 | 2.1 | 45.5 | 9.2 |
| | | | SD | 3.9 | 5.9 | | 7.3 | |
| Stiffness | | N/mm | Mean | 5259.1 | 5308.6 | 0.9 | 6194.6 | 17.8 |
| | | | SD | 1025.2 | 1223.8 | | 1387.5 | |
| Modulus | | MPa | Mean | 2144.7 | 2102.6 | −2.0 | 2197.6 | 2.5 |
| | | | SD | 290.3 | 500.0 | | 407.4 | |
| AUC | | N-mm | Mean | 20.8 | 21.5 | 3.5 | 25.4 | 22.1 |
| | | | SD | 4.4 | 4.1 | | 3.5 | |
| Toughness | | MPa | Mean | 0.9 | 0.9 | 2.0 | 1.1 | 20.6 |
| | | | SD | 0.1 | 0.1 | | 0.1 | |
| pQCT | Total | mm² | Mean | 7.5 | 7.7 | 2.3 | 8.1 | 7.4 |
| | Area | | SD | 0.8 | 0.6 | | 0.6 | |
| | Total | mg/mm | Mean | 5.3 | 5.6 | 5.9 | 5.9 | 12.4 |
| | BMC | | SD | 0.6 | 0.4 | | 0.5 | |
| | Total | mg/cm³ | Mean | 702.5 | 727.9 | 3.6 | 734.0 | 4.5 |
| | BMD | | SD | 25.9 | 38.3 | | 34.7 | |
| | Trabecular | mm² | Mean | 3.4 | 3.5 | 2.4 | 3.6 | 7.5 |
| | Area | | SD | 0.3 | 0.3 | | 0.3 | |
| | Trabecular | mg/mm | Mean | 1.5 | 1.7 | 9.0 | 1.8 | 15.1 |
| | BMC | | SD | 0.2 | 0.2 | | 0.3 | |
| | Trabecular | mg/cm³ | Mean | 455.5 | 486.9 | 6.9 | 486.4 | 6.8 |
| | BMD | | SD | 23.2 | 35.4 | | 60.6 | |

% = percent difference from Vehicle Control
Values in bold are significantly different from Group 1 values (Statistical analysis were performed on the least square mean)

TEXT TABLE 8a
Biomechanics - Summary Data Percent Difference from
Vehicle L3 + L4 Vertebral Bodies - Young Adults

| | | | | Vehicle | SAR7226 10 | | SAR7226 50 | |
|---|---|---|---|---|---|---|---|---|
| | | | | Control | mg/kg/day | | mg/kg/day | |
| | | Units | Group | Value | Value | % | Value | % |
| Peak Load | | N | Mean | 243.1 | 255.9 | 5.2 | 268.0 | 10.2 |
| | | | SD | 37.6 | 54.9 | | 30.3 | |
| Apparent Strength | | MPa | Mean | 33.3 | 34.4 | 3.5 | 38.7 | 16.4 |
| | | | SD | 3.9 | 6.7 | | 4.7 | |
| Yield Load | | N | Mean | 205.5 | 222.5 | 8.3 | 225.4 | 9.7 |
| | | | SD | 38.5 | 48.7 | | 26.9 | |
| Yield Stress | | MPa | Mean | 28.2 | 29.9 | 6.1 | 32.7 | 15.8 |
| | | | SD | 4.5 | 5.9 | | 4.2 | |
| Stiffness | | N/mm | Mean | 3581.0 | 4009.8 | 12.0 | 3951.0 | 10.3 |
| | | | SD | 545.6 | 968.9 | | 1055.6 | |
| Modulus | | MPa | Mean | 1308.3 | 1559.1 | 19.2 | 1613.3 | 23.3 |
| | | | SD | 218.2 | 437.6 | | 459.6 | |
| AUC | | N-mm | Mean | 16.0 | 14.4 | −9.8 | 16.3 | 2.1 |
| | | | SD | 5.6 | 4.5 | | 2.9 | |
| Toughness | | MPa | Mean | 0.8 | 0.7 | −19.6 | 0.9 | 1.5 |
| | | | SD | 0.3 | 0.2 | | 0.2 | |
| pQCT | Total | mm² | Mean | 7.3 | 7.4 | 1.7 | 6.9 | −5.1 |
| | Area | | SD | 0.9 | 0.7 | | 0.5 | |
| | Total | mg/mm | Mean | 4.5 | 4.9 | 8.5 | 4.7 | 2.7 |
| | BMC | | SD | 0.7 | 0.6 | | 0.4 | |
| | Total | mg/cm³ | Mean | 617.2 | 659.9 | 6.9 | 670.6 | 8.6 |
| | BMD | | SD | 32.9 | 35.7 | | 31.2 | |
| | Trabecular | mm² | Mean | 3.3 | 3.4 | 1.6 | 3.1 | −5.0 |
| | Area | | SD | 0.4 | 0.3 | | 0.2 | |
| | Trabecular | mg/mm | Mean | 1.4 | 1.6 | 10.5 | 1.4 | 2.7 |
| | BMC | | SD | 0.3 | 0.3 | | 0.2 | |
| | Trabecular | mg/cm³ | Mean | 424.0 | 461.6 | 8.9 | 461.9 | 8.9 |
| | BMD | | SD | 48.5 | 53.1 | | 35.3 | |

% = percent difference from Vehicle Control
Values in bold are significantly different from Group 1 values (Statistical analysis were performed on the least square mean)

Results Biomechanics

Test compound resulted in positive effects on bone mass and bone strength associated parameters at both dose levels in both rat populations, consistent with increase in trabecular bone noted histopathologically (young only). Strength parameters were positively affected at the lumbar spine (50 mg/kg/day).

The examples detailed below serve to illustrate the invention without, however, restricting it.

TABLE 9
Compounds of the formula I
I

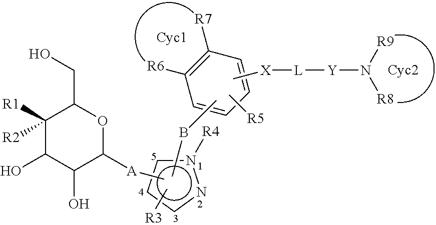

| Ex. | R1 | R2 | R3 | R4 | R5 | R6 | R7 | R8, R9 | A | B | X | L | Y | MS* | t_{R} [min] |
|---|---|---|---|---|---|---|---|---|---|---|---|---|---|---|---|
| 1 | H | F | i-Pr | H | H | H | H | CH₂CH₂NHCH₂CH₂ | O | CH₂ | — | —CH═CHCH₂— | — | 505.47 | 1.19 |
| 2 | H | F | i-Pr | H | H | H | H | H; CH₂CH₂CH₂CH₃ | O | CH₂ | — | —CH₂CH₂CH₂— | CO | 522.57 | 1.74 |
| 3 | H | F | i-Pr | H | H | H | H | H; CH₂CH₂CONH₂ | O | CH₂ | — | —CH═CHCH₂— | CO | 535.44 | 1.15 |
| 4 | H | F | i-Pr | H | H | H | H | H; CH₂CH₂CONH₂ | O | CH₂ | — | —CH₂CH₂CH₂— | CO | 537.44 | 1.13 |
| 5 | H | F | i-Pr | H | H | H | H | H; CH₂CONH₂ | O | CH₂ | — | —CH₂CH₂CH₂— | CO | 523.38 | 1.15 |
| 6 | H | F | CH₃ | H | H | H | H | H; CH₂CH₂CONH₂ | O | CH₂ | — | —CH₂CH₂CH₂— | CO | 509.33 | 1.02 |
| 7 | H | F | i-Pr | H | H | H | H | H; CH₂CH₂CONH₂ | O | CH₂ | — | —CH₂CH₂— | CO | 523.42 | 1.36 |
| 8 | H | F | i-Pr | H | H | H | H | H; CH₂CONH₂ | O | CH₂ | — | —CH₂CH₂— | CO | 509.29 | 1.08 |
| 9 | H | F | i-Pr | H | H | H | H | H; CH[CH₂OH]CONH₂ | O | CH₂ | — | —CH₂CH₂CH₂— | CO | 552.29 | 1.13 |
| 10 | H | F | CH₃ | H | H | H | H | H; CH[CH₂OH]CONH₂ | O | CH₂ | — | —CH₂CH₂CH₂— | CO | 525.31 | 1.02 |
| 11 | H | F | CH₃ | H | H | H | H | CH₂CH₂N[CH₂CH₂OH]CH₂CH₂ | O | CH₂ | — | —CH₂CH₂CH₂— | CO | 551.30 | 0.95 |
| 12 | H | F | i-Pr | H | H | H | H | CH₂CH₂N[CH₃]CH₂CH₂ | O | CH₂ | — | —CH₂CH₂CH₂— | CO | 549.30 | 1.07 |
| 13 | H | F | i-Pr | H | H | H | H | CH₂CH₂CH₂CH₂CH₂ | O | CH₂ | — | —CH₂CH₂CH₂— | CO | 534.54 | 1.77 |
| 14 | H | F | i-Pr | H | H | H | H | CH₂CH₂CH₂CH₂CH₂CH₂ | O | CH₂ | — | —CH₂CH₂CH₂— | CO | 548.56 | 1.83 |
| 15 | H | F | i-Pr | H | H | H | H | CH₂CH₂CH₂CH₂ | O | CH₂ | — | —CH₂CH₂CH₂— | CO | 520.52 | 1.67 |
| 16 | H | F | i-Pr | H | H | H | H | CH₂CH₂NHCH₂CH₂ | O | CH₂ | — | —CH₂CH₂CH₂— | CO | 535.32 | 1.06 |
| 17 | H | F | i-Pr | H | H | H | H | H; CH₂CH₂OH | O | CH₂ | — | —CH₂CH₂— | CO | 496.43 | 1.37 |
| 18 | H | F | i-Pr | H | H | H | H | H; C[CH₃]₂CH₂OH | O | CH₂ | — | —CH₂CH₂— | CO | 524.26 | 1.34 |
| 19 | H | F | i-Pr | H | H | H | H | H; C[CH₃]₂CH₂OH | O | CH₂ | — | —CH₂CH₂CH₂— | CO | 538.28 | 1.26 |
| 20 | H | F | i-Pr | H | H | H | H | H; CH₂CH₂OH | O | CH₂ | — | —CH═CH— | CO | 494.28 | 1.10 |
| 21 | H | F | i-Pr | H | H | H | H | H; C[CH₂OH]₃ | O | CH₂ | — | —CH₂CH₂CH₂— | CO | 570.33 | 1.14 |
| 22 | H | F | i-Pr | H | H | H | H | H; CH₂CH₂CH₂NH₂ | O | CH₂ | — | —CH₂CH₂CH₂— | CO | 522.52 | 1.74 |
| 23 | H | F | i-Pr | H | H | H | H | H; 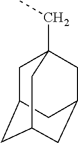 | O | CH₂ | — | —CH₂CH₂CH₂— | CO | 614.45 | 2.11 |
| 24 | H | F | i-Pr | H | H | H | H | H; 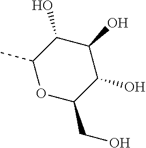 | O | CH₂ | — | —CH₂CH₂CH₂— | CO | 628.25 | 1.07 |
| 25 | H | F | i-Pr | H | H | H | H | CH₂CH₂N[SO₃H]CH₂CH₂ | O | CH₂ | — | —CH₂CH₂CH₂— | CO | 615.42 | 1.64 |
| 26 | H | F | i-Pr | H | H | H | H | H; CH₂CH₂CH₂NHSO₃H | O | CH₂ | — | —CH₂CH₂CH₂— | CO | 603.41 | 1.56 |
| 27 | H | F | i-Pr | H | H | H | H | H; CH₂CH₂OSO₃H | O | CH₂ | — | —CH₂CH₂CH₂— | CO | 588.50 | 1.60 |
| 28 | H | F | i-Pr | H | H | H | H | H; C[CH₃]₂CH₂OSO₃H | O | CH₂ | — | —CH₂CH₂CH₂— | CO | 616.52 | 1.61 |
| 29 | H | F | i-Pr | H | H | H | H | CH₂CH₂OCH₂CH₂ | O | CH₂ | — | —CH₂CH₂CH₂— | CO | 536.48 | 1.58 |
| 30 | H | F | i-Pr | H | H | H | H | H; C[CH₃]₂CH₂CH₃ | O | CH₂ | — | —CH₂CH₂CH₂— | CO | 536.54 | 1.84 |
| 31 | H | F | CH₃ | H | H | H | H | H; CH₂CH₂CH₃ | O | CH₂ | — | —CH₂CH₂CH₂— | NHCO | 494.12 | 2.77 |
| 32 | H | F | i-Pr | H | H | H | H | H; H | O | CH₂ | CO | —NHCH₂CH₂— | — | 494.97 | 0.97 |
| 33 | H | F | i-Pr | H | H | H | H | H; H | O | CH₂ | CO | —NHCH₂— | — | 481.19 | 1.02 |
| 34 | H | F | CF₃ | H | H | H | H | H; H | O | CH₂ | CO | —NHCH₂CH₂— | — | 521.16 | 1.00 |
| 35 | H | F | CF₃ | H | H | H | H | H; H | O | CH₂ | CO | —NHCH₂— | — | 507.16 | 1.20 |
| 36 | H | F | i-Pr | H | H | H | H | CH₂CH₂N[CH₃]CH₂CH₂ | O | CH₂ | — | —CH₂CH₂CH₂— | NHCO | n.d. | n.d. |
| 37 | H | F | i-Pr | H | H | H | H | H; C[CH₃]₂CH₂OH | O | CH₂ | — | —CH₂CH₂CH₂— | NHCO | n.d. | n.d. |

The linkages are indicated in the description of the examples in the experimental section.

Gradient for LCMS: acetonitrile+0.05% TFA:water+0.05% TFA: 5:95 (0 min) to 95:5 (2.5 min) to 95:5 (3 min); column: YMC J'shere 33×2, 4 M, 1.3 mL/min flow (gradient 1).

Further LCMS gradients differing therefrom are indicated in the experimental section:

Gradient 2: 0 min 96% H₂O (0.05% TFA) to 2.0 min-95% MeCN, then to 2.4 min 95% MeCN; then to 4% MeCN by 2.45 min.; 1 mL/min; 110-1000 MW; 0.4 L (YMC J'sphere ODS H80 20×2 1.4.

Gradient 3: 0 min 95% H₂O (5 mmol ammonium acetate) to 3.5 min at 95% MeCN, then for 2 min 95% ACN; then in one minute to 5°)/MeCN; 0.5 mL/min; 115-1000 MW; 1 L (Merck Purospher 3, 2×55 mm),

TABLE 10
Compounds of the formula II
I

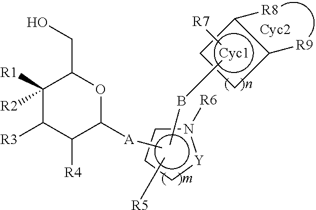

| Bsp | R1 | R2 | R3 | R4 | R5 | R6 | R7 | R8, R9 | A | B | Cyc1 | X | Y | m | n | MS* |
|---|---|---|---|---|---|---|---|---|---|---|---|---|---|---|---|---|
| 1 | H | F | OH | OH | H | — | 4-O—CH₃ | H, H | O | CH₂ | Ph | C | S | 1 | 3 | ok |
| 2 | H | F | OH | OH | —CH═CH—CH═CH— | | 4-O—CH₃ | H, H | O | CH₂ | Ph | C | S | 1 | 3 | ok |
| 3 | F | H | OH | OH | OH | — | 4-O—CH₃ | H, H | O | CH₂ | Ph | C | S | 1 | 3 | ok |
| 4 | H | OH | F | OH | OH | — | 4-O—CH₃ | H, H | O | CH₂ | Ph | C | S | 1 | 3 | ok |
| 5 | H | F | OH | OH | CF₃ | H | 4-O—CH₃ | H, H | O | CH₂ | Ph | N | N | 1 | 3 | ok |
| 6 | F | H | OH | OH | CF₃ | H | 4-O—CH₃ | H, H | O | CH₂ | Ph | N | N | 1 | 3 | ok |
| 7 | H | F | OH | OH | CH₃ | H | 4-F | H, H | O | CH₂ | Ph | N | N | 1 | 3 | ok |
| 8 | H | F | OH | OH | CH₃ | H | 2-Cl | 4-Cl, H | O | CH₂ | Ph | N | N | 1 | 3 | ok |
| 9 | H | F | OH | OH | CH₃ | CH₃ | 4-F | H, H | O | CH₂ | Ph | N | N | 1 | 3 | ok |
| 10 | H | F | OH | OH | CH₃ | CH₃ | 2-Cl | 4-Cl, H | O | CH₂ | Ph | N | N | 1 | 3 | ok |
| 11 | H | F | OH | OH | H | — | 4-CH₂—CH₃ | H, H | O | CH₂ | Ph | C | S | 1 | 3 | ok |
| 12 | F | H | OH | OH | H | — | 4-CH₂—CH₃ | H, H | O | CH₂ | Ph | C | S | 1 | 3 | ok |
| 13 | H | F | OH | OH | H | — | 4-O—CH₃ | H, H | O | CONHCH₂ | Ph | C | S | 1 | 3 | ok |
| 14 | H | F | OH | OH | H | — | 4-O—CF₃ | H, H | O | CONHCH₂ | Ph | C | S | 1 | 3 | ok |
| 15 | H | F | OH | OH | CH₃ | — | 4-O—CH₃ | H, H | O | CH₂ | Ph | C | S | 1 | 3 | ok |
| 16 | H | F | OH | OH | H | — | H | —CH═CH—CH═CH— | O | CH₂ | thio- | C | S | 1 | 2 | ok |
| | | | | | | | | | | | phene | | | | | |
| 17 | H | F | OH | OH | H | — | 4-CH₃ | H, H | O | CH₂ | Ph | C | S | 1 | 3 | ok |
| 18 | H | F | OH | OH | H | — | 2-CH₃ | H, H | O | CH₂ | Ph | C | S | 1 | 3 | ok |
| 19 | H | F | OH | OH | H | — | 4-I | H, H | O | CH₂ | Ph | C | S | 1 | 3 | ok |
| 20 | F | F | OH | OH | CF₃ | H | 4-O—CH₃ | H, H | O | CH₂ | Ph | N | N | 1 | 3 | ok |
| 21 | H | F | OH | OH | H | — | 3-Me | H, H | O | CH₂ | Ph | C | S | 1 | 3 | ok |
| 22 | H | F | OH | OH | H | — | 4-Cl | H, H | O | CH₂ | Ph | C | S | 1 | 3 | ok |
| 23 | H | F | OH | OH | H | — | 4-F | H, H | O | CH₂ | Ph | C | S | 1 | 3 | ok |
| 24 | H | F | OH | OH | H | — | H | —CH═CH—CH═CH— | O | CH₂ | Ph | C | S | 1 | 3 | ok |
| 25 | H | F | OH | OH | H | — | 4-OCF₃ | H, H | O | CH₂ | Ph | C | S | 1 | 3 | ok |
| 26 | H | F | OH | OH | H | — | 4-Br | H, H | O | CH₂ | Ph | C | S | 1 | 3 | ok |
| 27 | H | F | OH | OH | H | — | 4-CH(CH₃)₂ | H, H | O | CH₂ | Ph | C | S | 1 | 3 | ok |
| 28 | H | F | OH | OH | H | — | H | —CH═CH—C(OMe)═CH— | O | CH₂ | Ph | C | S | 1 | 3 | ok |
| 29 | H | F | OH | OH | H | — | H | —CH═CH—O— | O | CH₂ | Ph | C | S | 1 | 3 | ok |
| 30 | H | F | OH | OH | CH₃ | H | 2-F | H, H | O | CH₂ | Ph | N | N | 1 | 3 | ok |
| 31 | H | F | OH | OH | CH₃ | H | 4-Cl | H, H | O | CH₂ | Ph | N | N | 1 | 2 | ok |

The indication “MS is ok” means that a mass spectrum or HPLC/MS was recorded and the molecular peak M+1 (MH⁺) and/or M+18 (MNH₄⁺) and/or M+23 (MNa⁺) was detected therein. The linkages are indicated in the description of the examples in the experimental part.

TABLE 11
Compounds of the formula IIIb
Ib

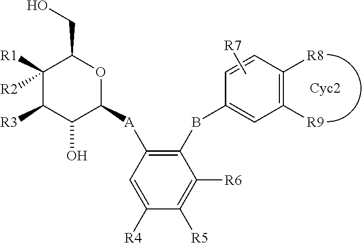

| Ex. | R1 | R2 | R3 | R4, R5, R6 | R7 | R8, R9 | A | B | Cyc1 | Cyc2 | MS* |
|---|---|---|---|---|---|---|---|---|---|---|---|
| 1 | H | F | OH | H, H, H | H | O—CH₃, H | O | CH₂ | Phenyl | | Ok |
| 2 | H | F | OH | H, H, H | H | H, H | O | CH₂ | Phenyl | | Ok |
| 3 | F | H | OH | H, H, H | H | O—CH₃, H | O | CH₂ | Phenyl | | Ok |
| 4 | H | F | OH | CH₃, H, OH | H | —CH═CH—O— | O | CO—CH₂—CH₂ | Phenyl | Furenyl | Ok |
| 5 | F | H | OH | CH₃, H, OH | H | —CH═CH—O— | O | CO—CH₂—CH₂ | Phenyl | Furenyl | Ok |
| 6 | F | H | OH | CH₃, H, OH | H | —CH₂—CH₂—O | O | CO—CH₂—CH₂ | Phenyl | Furanyl | Ok |
| 7 | H | F | OH | CH₃, H, OH | H | O—CH₃, H | O | CO—CH₂—CH₂ | Phenyl | | Ok |
| 8 | H | F | OH | CH₃, H, OH | H | OH, H | O | CO—CH₂—CH₂ | Phenyl | | Ok |
| 9 | H | F | OH | OH, H, OH | H | OH, H | O | CO—CH₂—CH₂ | Phenyl | | Ok |
| 10 | F | H | OH | OH, H, OH | H | OH, H | O | CO—CH₂—CH₂ | Phenyl | | Ok |
| 11 | H | F | OH | H, H, OH | H | H, H | O | CO—NH—CH₂ | Phenyl | | Ok |
| 12 | F | H | OH | H, H, OH | H | O—CH₃, H | O | CO—NH—CH₂ | Phenyl | | Ok |
| 13 | H | F | OH | CH₃, H, OH | H | O—CH₃, H | O | CO—NH—CH₂ | Phenyl | | Ok |
| 14 | F | H | OH | CH₃, H, OH | H | O—CH₃, H | O | CO—NH—CH₂ | Phenyl | | Ok |
| 15 | H | F | OH | CH₃, H, OH | H | CH₂CH₃, H | O | CO—CH₂—CH₂ | Phenyl | | Ok |
| 16 | H | F | OH | CH₃, H, OH | H | CH₃, H | O | CO—CH₂—CH₂ | Phenyl | | Ok |
| 17 | H | F | OH | CH₃, H, OH | H | OCF₃, H | O | CO—CH₂—CH₂ | Phenyl | | Ok |
| 18 | H | F | OH | CH₃, H, OH | H | Cl, H | O | CO—CH₂—CH₂ | Phenyl | | Ok |
| 19 | F | F | OH | H, H, H | H | H, H | O | CH₂ | Phenyl | | Ok |
| 20 | H | F | OH | H, H, H | H | CH₂CH₃, H | O | CH₂ | Phenyl | | Ok |
| 21 | H | F | OH | OCH₃, H, H | H | O—CH₃, H | O | CH₂ | Phenyl | | Ok |
| 22 | H | F | OH | H, F, H | H | O—CH₃, H | O | CH₂ | Phenyl | | Ok |
| 23 | H | F | OH | H, OCH₃, H | H | CH₂CH₃, H | O | CH₂ | Phenyl | | Ok |
| 24 | H | F | OH | H, H, H | H | CH₂CH₃, H | O | O | Phenyl | | Ok |

*The indication “MS is ok” means that a mass spectrum or HPLC/MS was recorded and the molecular peak + 1 (MH⁺) and/or M + 18 (MNH₄⁺) and/or M + 23 (MNa⁺) was detected therein

The invention further relates to processes for preparing the compounds of the general formula I.

### EXPERIMENTAL SECTION

The preparation of the examples is described in detail below. The compounds of the invention can be obtained analogously or in accordance with the processes described in WO 0414932 and WO 0418491.

### Preparation of Compounds of Formula I

### Reaction Scheme

### Synthesis of the -Bromoglycoside 4

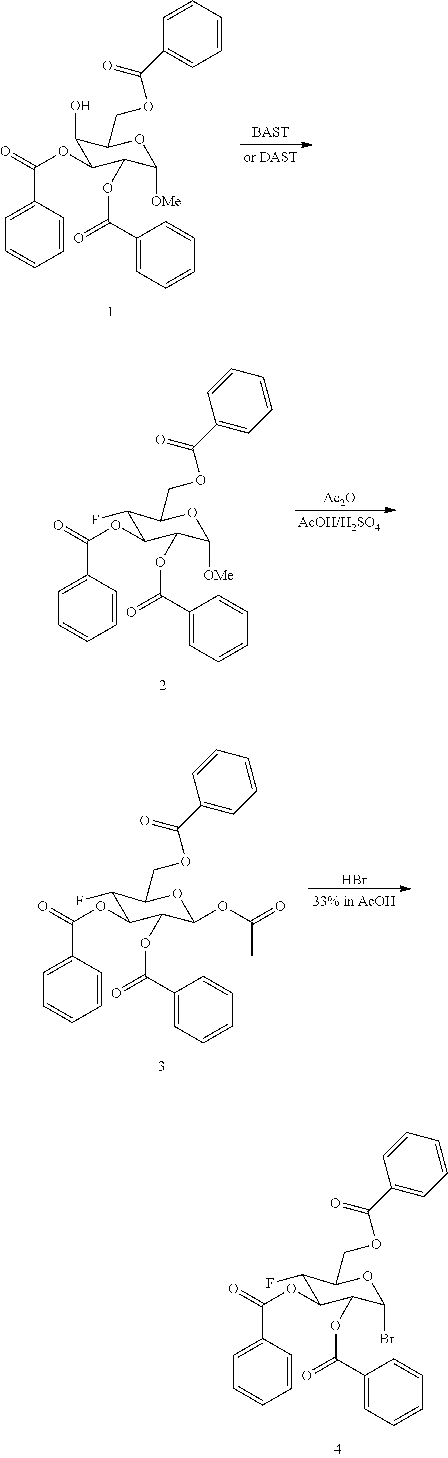

### Methyl 2,3,6-tri-O-benzoyl-4-fluoro-4-deoxy- -D-glucopyranoside (2)

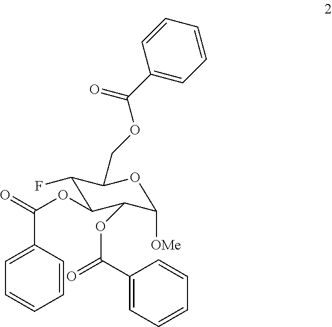

3.0 g of methyl 2,3,6-tri-O-benzoyl- -D-galactopyranoside (Reist et al., J. Org. Chem. 1965, 30, 2312) are introduced into dichloromethane and cooled to −30° C. Then 3.06 ml of [bis(2-methoxyethyl)amino]sulfur trifluoride (BAST) are added dropwise. The reaction solution is warmed to room temperature and stirred for 12 h. The mixture is diluted with dichloromethane, and the organic phase is extracted with H₂O, NaHCO₃ solution and saturated NaCl solution. The organic phase is dried over Na₂SO₄ and concentrated. The crude product is crystallized from ethyl acetate and heptane. 1.95 g of the product 2 are obtained as a colorless solid. C₂₈H₂₅FO₈ (508.51) MS (ESI⁺) 526.18 (M+NH₄⁺). Alternatively, the reaction can also be carried out using 2.8 eq. of diethylaminosulfur trifluoride (DAST); in this case, the reaction solution is refluxed for 18 h after the addition. The working up takes place in analogy to the above description.

### 1-O-Acetyl-2,3,6-tri-O-benzoyl-4-fluoro-4-deoxyglucose (3)

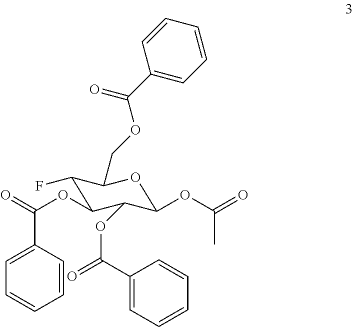

12.0 g of compound methyl 2,3,6-tri-O-benzoyl-4-fluoro-4-deoxy- -D-glucopyranoside are suspended in 150 ml of acetic anhydride. 8.4 ml of conc. sulfuric acid are mixed with 150 ml of glacial acetic acid and added to the mixture while cooling in ice. The mixture stirs at room temperature for 60 h. The mixture is poured into NaHCO₃ solution, and this solution is extracted with dichloromethane. The organic phase is extracted with NaCl solution, dried with Na₂SO₄ and concentrated. The residue is recrystallized from ethyl acetate/heptane. 5.97 g of the product 3 are obtained as a colorless solid.

C₂₉H₂₅FO₉ (536.52) MS (ESI⁺) 554.15 (M+NH₄⁺)

### 1-Bromo-4-deoxy-4-fluoro-2,3,6-tri-O-benzoyl-alpha-D-glucose (4)

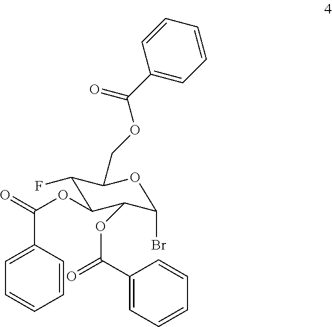

1.44 g of 1-O-acetyl-2,3,6-tri-O-benzoyl-4-fluoro-4-deoxyglucose are dissolved in 20 ml of hydrobromic acid in glacial acetic acid (33%) and stirred at room temperature. After 5 hours, the mixture is poured into ice-water, and the aqueous phase is extracted three times with dichloromethane. The collected organic phase is washed with saturated sodium chloride solution, dried over sodium sulfate and evaporated to dryness. The crude product is filtered through a silica gel column with ethyl acetate/heptane 70:30. 1.40 g of the product 4 are obtained as a colorless solid. C₂₇H₂₂BrFO₇ (557.37) MS (ESI⁺) 574.05/576.05 (M+NH₄⁺)

### Reaction Scheme I

### Synthesis of Example 1

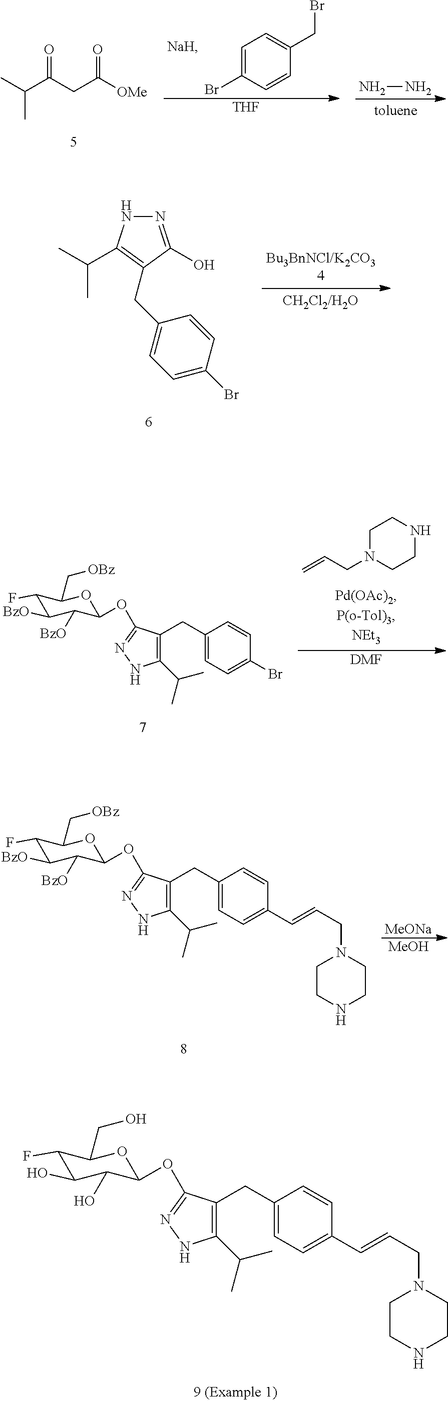

### 4-(4-Bromobenzyl)-5-isopropylpyraz-3-ol (6)

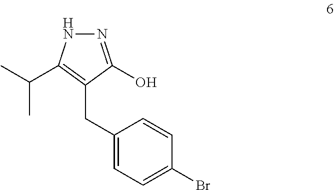

15.2 g of methyl isobutyrylacetate (5) are added to a suspension of sodium hydride (60%, 3.85 g) in 250 ml of tetrahydrofuran while cooling in ice. A solution of 20.0 g of 4-bromobenzyl bromide in 100 ml of THF is then added and the mixture is stirred at room temperature for 48 h. After addition of 300 ml of H₂O and 300 ml of EtOAc, the organic phase is dried over MgSO₄ and the solvent is stripped off in a rotary evaporator. The resulting crude product is dissolved in 120 ml of toluene, mixed with hydrazine hydrate (8.01 g) and heated under reflux with a water trap for 12 h. The reaction mixture is concentrated to a volume of 50 ml and cooled to 0° C. of the crystallized product is filtered off with suction and washed with heptane. 10.8 g of the compound 6 are obtained as a pale yellow solid. C₁₃H₁₅BrN₂O (295.18) MS (ESI⁺294.04 (M+H⁺).

### Compound 7

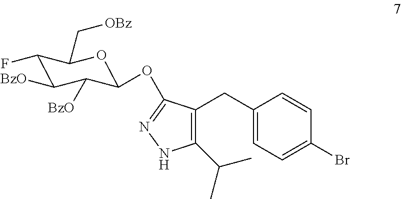

530 mg of 4-(4-bromobenzyl)-5-isopropylpyraz-3-ol (6) and 1.50 g of bromide 4 are dissolved in 50 ml of methylene chloride. To this solution are successively added 1.86 g of potassium carbonate, 91 mg of benzyltriethylammonium bromide and 0.8 ml of water, and it is then stirred at room temperature for 24 hours. The reaction solution is transferred into a separating funnel and washed successively with water and saturated sodium chloride solution. The organic phase is dried over magnesium sulfate and concentrated in a rotary evaporator. The crude product is separated by chromatography on silica gel (EtOAc/heptane). 193 mg of 7 are obtained as a colorless solid. C₄₀H₃₆BrFN₂O₈ (771.6) MS (ESI⁺) 773.1 (M+H⁺).

### Compound 8

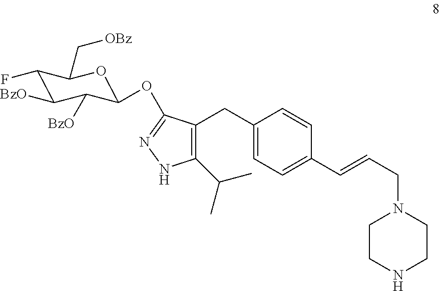

193 mg of the glycoside 7 are dissolved in 1.25 ml of DMF, and 2.3 mg of Pd(OAc)₂, 6.09 mg of tri-o-tolylphosphine, 0.25 ml of triethylamine and 84.6 ml of 1-allylpiperazine are added. The reaction mixture is heated in an oil bath at 100° C. for 18 h. The solvent is removed in a rotary evaporator, and the crude product is purified by chromatography on silica gel (EtOAc/MeOH). 117 mg of the compound 8 are obtained as a colorless wax. C₄₇H₄₉FN₄O₈ (816.9) MS (ESI⁺) 817.05 (M+H⁺).

### Compound 9

### Example 1

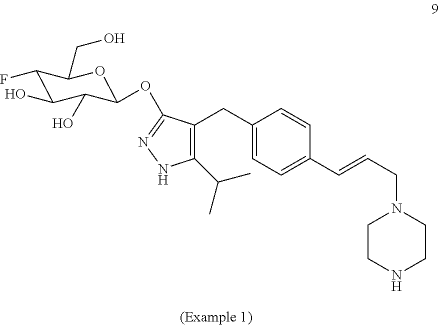

98 mg of the glycoside 8 are taken up in 4 ml of a mixture of methanol/water/triethylamine (3:3:1) and stirred at room temperature for 48 h. The reaction mixture is concentrated in a rotary evaporator, and the residue is purified by chromatography on silica gel (methylene chloride/methanol/conc. ammonia). 34 mg of the compound 9 are obtained as a colorless solid. C₂₆H₃₇FN₄O₅ (504.61) MS (ESI⁺) 505.47 (M+H⁺).

### Reaction Scheme II

### Synthesis of Example 2

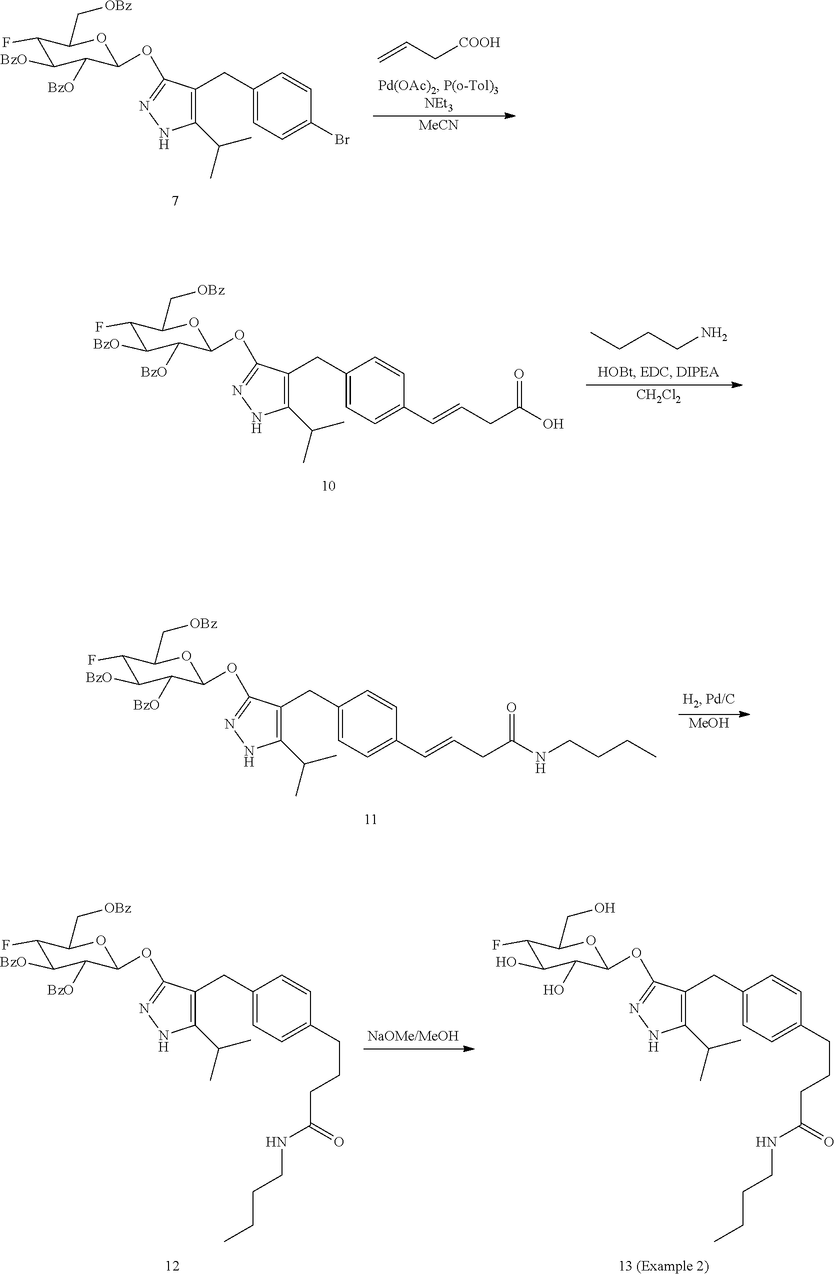

### Compound 10

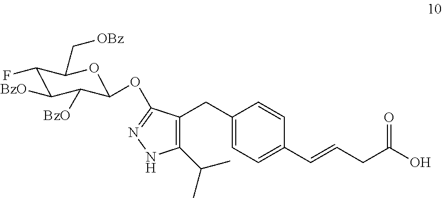

7.20 g of the glycoside 7 are dissolved in 109 ml of acetonitrile, and 41.9 mg of Pd(OAc)₂, 113.6 mg of tri-o-tolylphosphine, 39.2 ml of triethylamine and 1.04 g of vinyl acetic acid are added. The reaction mixture is heated under reflux for 60 h. The solvent is removed in a rotary evaporator, and the crude product is purified by chromatography on silica gel (CH₂Cl₂/MeOH/conc. ammonia=30/5/1). 6.18 g of the compound 10 are obtained as a colorless wax. C₄₄H₄₁FN₂O₁₀ (776.8).

### Compound 11

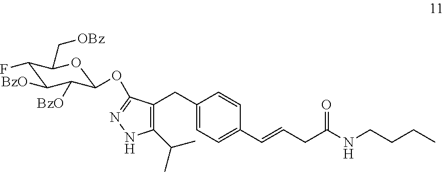

100 mg of compound 10 are dissolved in 4.00 ml of dichloromethane, and 9.41 mg of n-butylamine, 119.8 mg of diisopropylethylamine, 26.1 mg of 1-hydroxybenzotriazole and 30 mg of N-(3-dimethylaminopropyl)-N-ethylcarbodiimide are added. The reaction mixture is stirred at 20° C. for 16 h. The solution is washed successively with in each case 5 ml of NaHCO₃ solution, 5 ml of 0.2M hydrochloric acid and 5 ml of saturated NaCl solution. The solvent is removed in a rotary evaporator, and the crude product is converted without further purification into compound 12.

C₄₈H₅₀FN₂₃O₉ (831.9).

### Compound 12

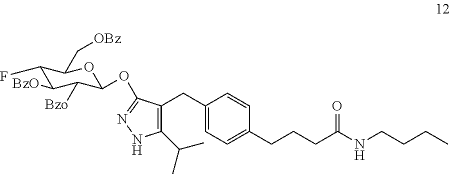

82 mg of compound 11 are dissolved in 5.00 ml of methanol, and 10.5 mg of palladium on activated carbon (10%) are added. The reaction mixture is stirred under an atmosphere of 1 bar of H₂ for 16 h. Palladium on carbon is filtered off, and the solvent is removed in a rotary evaporator. Further purification of the crude product on silica gel is unnecessary. 72 mg of the desired compound 12 are obtained as a colorless wax. C₄₈H₅₂FN₂₃O₉ (834.0).

### Compound 13

### Example 2

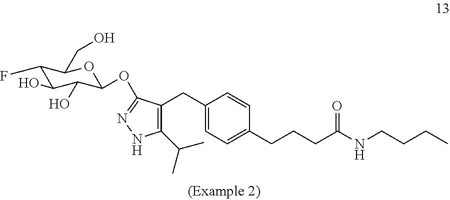

72 mg of the glycoside 12 are dissolved in 10 ml of methanol, and 1.72 ml of a 2M methanolic sodium methoxide solution are added. The reaction mixture is stirred at 20° C. for 4 h, and 46.2 mg of ammonium chloride are added. The solvent is removed in a rotary evaporator, and the crude product is purified on silica gel (initially with ethyl acetate/heptane=5/1; subsequently methylene chloride/methanol/conc. ammonia=30/5/1). 24 mg of the compound 13 are obtained as a colorless solid. C₂₇H₄₀FN₃O₉ (521.63): MS (ESI⁺) 522.57 (M+H⁺).

### Compound 14

### Example 3

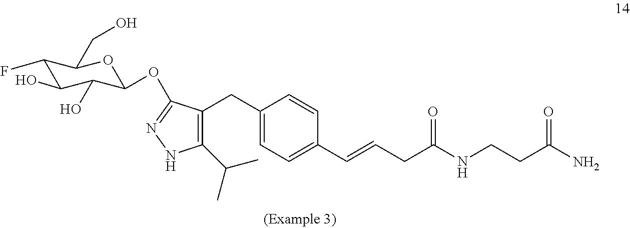

Compound 14 is synthesized in analogy to the synthesis route described for compound 13 (example 2). Starting from the glycoside 10, which is, however, reacted not with n-butylamine but with 3-aminopropionamide hydrochloride, and without carrying out the subsequent hydrogenation, compound 14 is obtained as a colorless solid. C₂₆H₃₅FN₄O₇ (534.6): MS (ESI⁺) 535.44 (M+H⁺).

### Compound 15

### Example 4

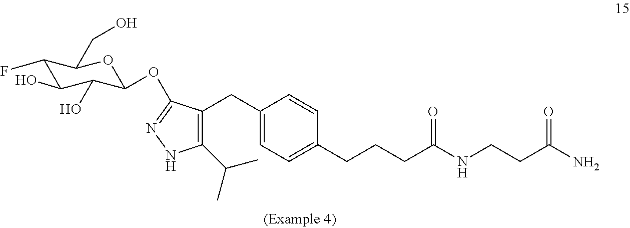

Compound 15 is synthesized in analogy to the synthesis route described for compound 13 (example 2). Starting from the glycoside 10, which is, however, reacted not with n-butylamine but with 3-aminopropionamide hydrochloride, compound 15 is obtained as a colorless solid. C₂₆H₃₇FN₄O₇ (536.6): MS (ESI⁺) 537.44 (M+H⁺).

### Compound 16

### Example 5

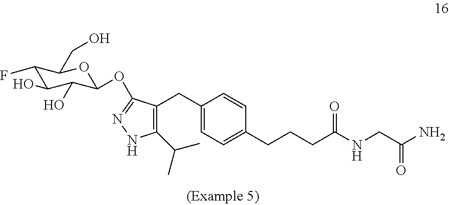

Compound 16 is synthesized in analogy to the synthesis route described for compound 13 (example 2). Starting from the glycoside 10, which is, however, reacted not with n-butylamine but with glycinamide hydrochloride, compound 16 is obtained as a colorless wax. C₂₅H₃₅FN₄O₇ (522.6): MS (ESI⁺) 523.38 (M+H⁺).

### Compound 17

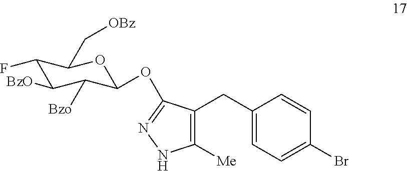

Compound 17 is synthesized in analogy to the synthesis route described for compound 7 (scheme I). However, ethyl acetoacetate is used as starting material instead of methyl isobutyrylacetate. Compound 17 is obtained as a colorless solid. C₃₈H₃₂BrFN₂O₈ (743.6).

### Compound 18

### Example 6

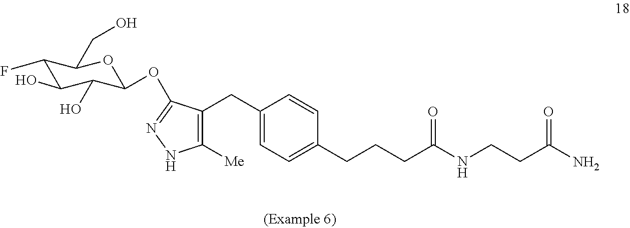

Compound 18 is synthesized in analogy to the synthesis described for compound 15 (example 4). However, the glycoside 17 is used as starting material instead of glycoside 10. Compound 18 is obtained as a colorless wax. C₂₄H₃₃FN₄O₇ (508.6): MS (ESI⁺) 509.33 (M+H⁺).

### Compound 19

### Example 7

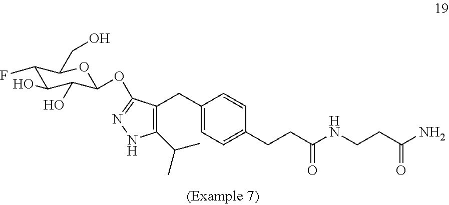

Compound 19 is synthesized in analogy to the synthesis described for compound 15 (example 4). However, the bromo compound 7 is reacted with acrylic acid instead of vinylacetic acid. Compound 19 is obtained as a colorless wax. C₂₅H₃₅FN₄O₇ (522.6): MS (ESI⁺) 523.42 (M+H⁺).

### Compound 20

### Example 8

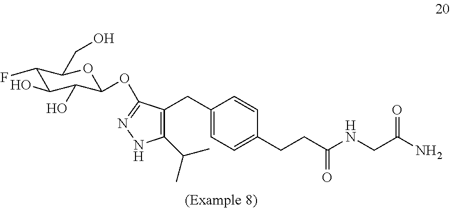

Compound 20 is synthesized in analogy to the synthesis described for compound 19 (example 7). However, 3-aminopropionamide hydrochloride is replaced by glycinamide hydrochloride in the amide coupling. Compound 20 is obtained as a colorless wax. C₂₄H₃₃FN₄O₇ (508.6): MS (ESI⁺) 509.29 (M+H⁺).

### Compound 21

### Example 9

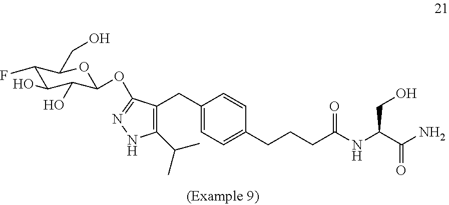

Compound 21 is synthesized in analogy to the synthesis route described for compound 13 (example 2). Starting from the glycoside 10, which is, however, reacted not with n-butylamine but with L-serinamide hydrochloride, compound 21 is obtained as a colorless solid. C₂₆H₃₇FN₄O₈ (552.6): MS (ESI⁺) 553.29 (M+H⁺).

### Compound 22

### Example 10

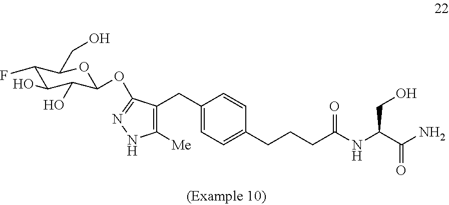

Compound 22 is synthesized in analogy to the synthesis described for compound 18 (example 6). Starting from the glycoside 17, which is, however, reacted not with 3-aminopropionamide hydrochloride but with L-serinamide hydrochloride, compound 22 is obtained as a colorless wax. C₂₄H₃₃FN₄O₈ (524.6): MS (ESI⁺) 525.31 (M+H⁺).

### Compound 23

### Example 11

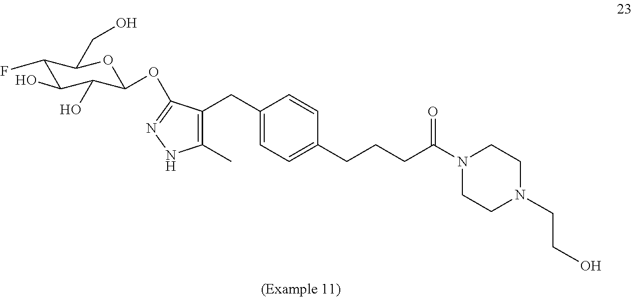

Compound 23 is synthesized in analogy to the synthesis route described for compound 18 (example 6). Starting from the glycoside 17, which is, however, reacted not with 3-aminopropionamide hydrochloride but with N-(2-hydroxyethyl)piperazine, compound 23 is obtained as a colorless wax. C₂₇H₃₉FN₄O₇ (550.6): MS (ESI⁺) 551.30 (M+H⁺).

### Compound 24

### Example 12

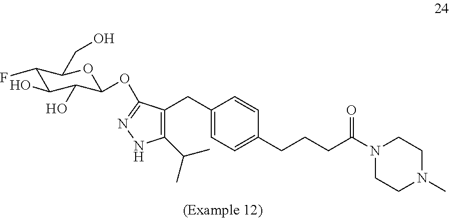

Compound 24 is synthesized in analogy to the synthesis route described for compound 13 (example 2). Starting from the glycoside 10, which is, however, reacted not with n-butylamine but with N-methylpiperazine, compound 24 is obtained as a colorless wax. C₂₈H₄₁FN₄O₆ (548.7): MS (ESI⁺) 549.30 (M+H⁺).

### Compound 25

### Example 13

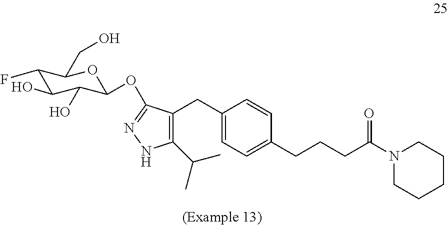

Compound 25 is synthesized in analogy to the synthesis route described for compound 13 (example 2). Starting from the glycoside 10, which is, however, reacted not with n-butylamine but with piperidine, compound 25 is obtained as a colorless wax.

C₂₈H₄₀FN₃O₆ (533.7): MS (ESI⁺) 534.54 (M+H⁺).

### Compound 26

### Example 14

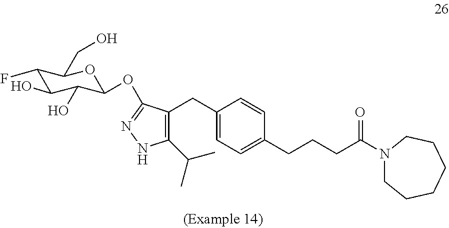

Compound 26 is synthesized in analogy to the synthesis route described for compound 13 (example 2). Starting from the glycoside 10, which is, however, reacted not with n-butylamine but with hexahydro-1H-azepine, compound 26 is obtained as a colorless wax.

C₂₉H₄₂FN₃O₆ (547.7): MS (ESI⁺) 548.56 (M+H⁺).

### Compound 27

### Example 15

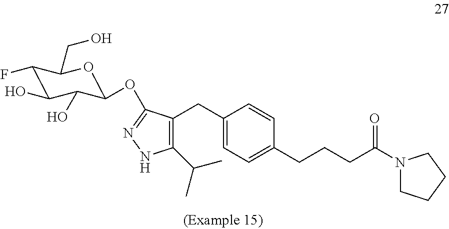

Compound 27 is synthesized in analogy to the synthesis route described for compound 13 (example 2). Starting from the glycoside 10, which is, however, reacted not with n-butylamine but with pyrrolidine, compound 27 is obtained as a colorless wax.

C₂₇H₃₈FN₃O₆ (519.6): MS (ESI⁺) 520.52 (M+H⁺).

### Compound 28

### Example 16

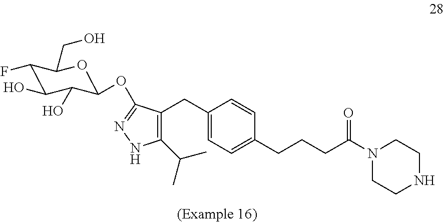

Compound 28 is synthesized in analogy to the synthesis route described for compound 13 (example 2). Starting from the glycoside 10, which is, however, reacted not with n-butylamine but with benzyl 1-piperazinecarboxylate, compound 28 is obtained as a colorless wax. C₂₇H₃₉FN₄O₆ (534.6): MS (ESI⁺) 535.32 (M+H⁺).

### Compound 29

### Example 17

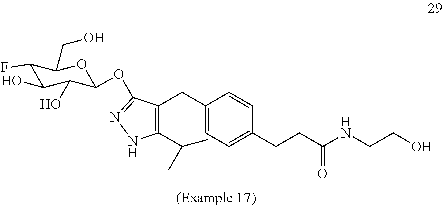

Compound 29 is synthesized in analogy to the synthesis described for compound 19 (example 7). However, 3-aminopropionamide hydrochloride is replaced by 2-aminoethanol in the amide coupling. Compound 29 is obtained as a colorless oil. C₂₄H₃₄FN₃O₇ (495.6): MS (ESI⁺) 496.43 (M+H⁺).

### Compound 30

### Example 18

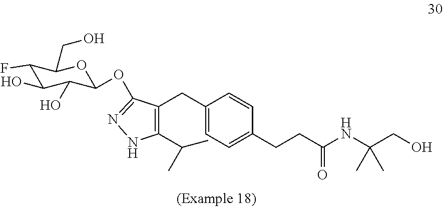

Compound 30 is synthesized in analogy to the synthesis described for compound 19 (example 7). However, 3-aminopropionamide hydrochloride is replaced by 2-amino-2-methyl-1-propanol in the amide coupling. Compound 30 is obtained as a colorless oil. C₂₆H₃₈FN₃O₇ (523.6): MS (ESI⁺) 524.26 (M+H⁺).

### Compound 31

### Example 19

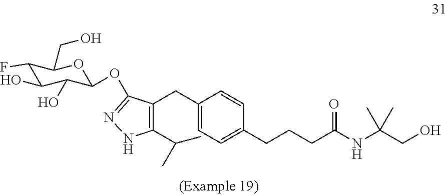

Compound 31 is synthesized in analogy to the synthesis route described for compound 13 (example 2). Starting from the glycoside 10, which is, however, reacted not with n-butylamine but with 2-amino-2-methyl-1-propanol, compound 31 is obtained as a colorless solid. C₂₇H₄₀FN₃O₇ (537.6): MS (ESI⁺) 538.28 (M+H⁺).

### Compound 32

### Example 20

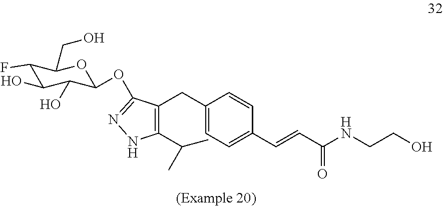

Compound 32 is synthesized in analogy to the synthesis described for compound 29 (example 17). However, the hydrogenation stage is not carried out. Compound 32 is obtained as a colorless wax. C₂₄H₃₂FN₃O₇ (493.6): MS (ESI⁺) 494.28 (M+H⁺).

### Compound 33

### Example 21

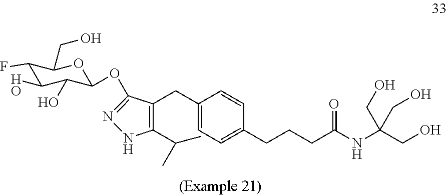

Compound 33 is synthesized in analogy to the synthesis route described for compound 13 (example 2). Starting from the glycoside 10, which is, however, reacted not with n-butylamine but with tris(hydroxymethyl)aminomethane, compound 33 is obtained as a colorless solid. C₂₇H₄₀FN₃O₉ (569.6): MS (ESI⁺) 570.33 (M+H⁺).

### Compound 34

### Example 22

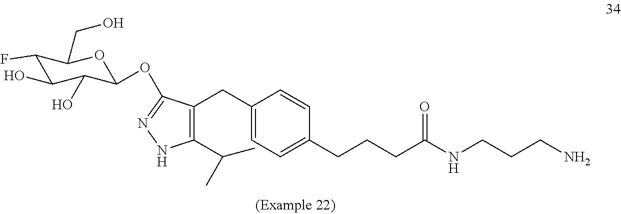

Compound 34 is synthesized in analogy to the synthesis route described for compound 13 (example 2). Starting from the glycoside 10, which is, however, reacted not with n-butylamine but with N-carbobenzoxy-1,3-diaminopropane hydrochloride, compound 34 is obtained as a colorless oil. C₂₆H₃₉FN₄O₆ (522.6): MS (ESI⁺) 522.52 (M+H⁺).

### Compound 35

### Example 23

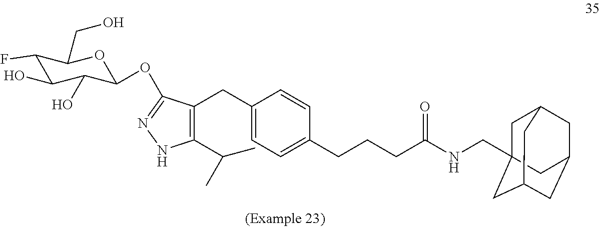

Compound 35 is synthesized in analogy to the synthesis route described for compound 13 (example 2). Starting from the glycoside 10, which is, however, reacted not with n-butylamine but with 1-adamantanemethylamine, compound 35 is obtained as a colorless wax.

C₃₄H₄₈FN₃O₆ (613.8): MS (ESI⁺) 614.45 (M+H⁺).

### Compound 36

### Example 24

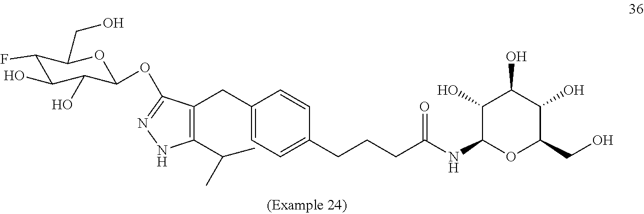

Compound 36 is synthesized in analogy to the synthesis route described for compound 13 (example 2). Starting from the glycoside 10, which is, however, reacted not with n-butylamine but with 2,3,4,6-tetra-O-acetyl-1-amino-1-deoxy-beta-D-glucose, compound 36 is obtained as a colorless oil. C₂₉H₄₂FN₃O₁₁ (627.7): MS (ESI⁺) 628.25 (M+H⁺).

### Compound 37

### Example 25

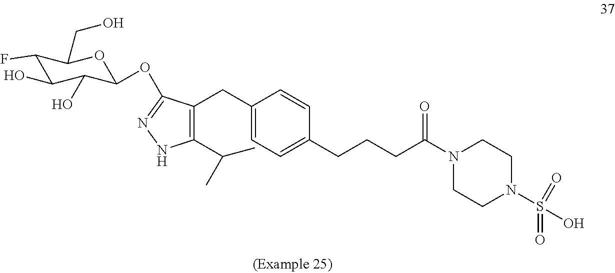

Compound 37 is synthesized in analogy to the synthesis route described for compound 28 (example 16). However, the last stage, the deprotection with sodium methanolate, is preceded by reaction with sulfur trioxide-triethylamine complex: this is done by dissolving 63.0 mg of the piperazine compound in 10.0 ml of methanol and, at 0° C., adding 202 mg of sulfur trioxide triethylamine complex and stirring at 0° C. for 2 h. The solvent is removed in a rotary evaporator, and the crude product is purified on silica gel (methylene chloride/methanol/conc. ammonia=30/5/1). 59 mg of the sulfate compound are obtained and converted, in analogy to the synthesis of compound 28 with sodium methoxide, into the compound 37, which is obtained as a colorless wax. C₂₇H₃₉FN₄O₉S (614.7): MS (ESI⁺) 615.42 (M+H⁺).

### Compound 38

### Example 26

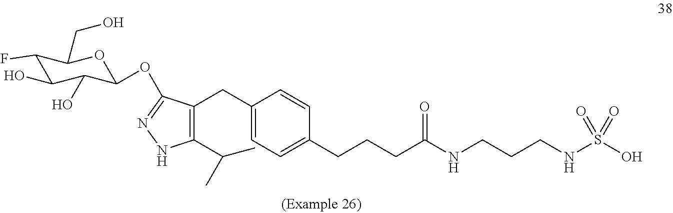

Compound 38 is synthesized in analogy to the synthesis route described for compound 37 (example 25). Starting from the glycoside 10, which is, however, reacted not with benzyl 1-piperazinecarboxylate but with N-carbobenzoxy-1,3-diaminopropane hydrochloride, compound 38 is obtained as a colorless wax. C₂₆H₃₉FN₄O₉S (602.7): MS (ESI⁺) 603.41 (M+H⁺).

### Compound 39

### Example 27

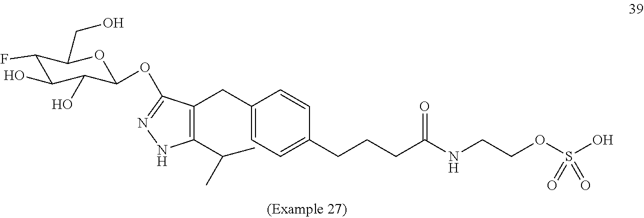

Compound 39 is synthesized in analogy to the synthesis route described for compound 37 (example 25). Starting from the glycoside 10, which is, however, reacted not with benzyl 1-piperazinecarboxylate but with 2-aminoethanol, compound 39 is obtained as a colorless wax.

C₂₅H₃₆FN₃O₁₀S (589.6): MS (ESI⁺) 588.50 (M⁺−H).

### Compound 40

### Example 28

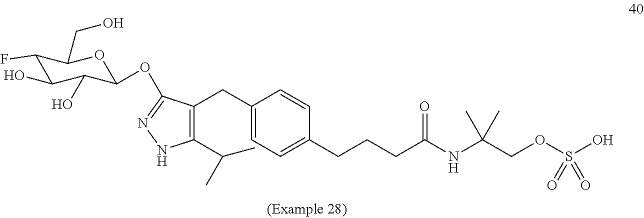

Compound 40 is synthesized in analogy to the synthesis route described for compound 37 (example 25). Starting from the glycoside 10, which is, however, reacted not with benzyl 1-piperazinecarboxylate but with 2-amino-2-methyl-1-propanol, compound 40 is obtained as a colorless wax. C₂₇H₄₀FN₃O₁₀S (617.7): MS (ESI⁺) 616.52 (M⁺−H).

### Compound 41

### Example 29

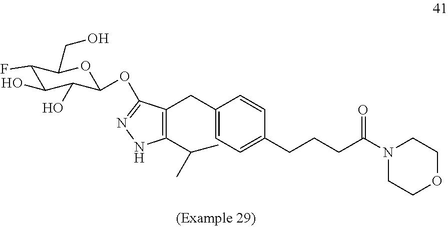

Compound 41 is synthesized in analogy to the synthesis route described for compound 13 (example 2). Starting from the glycoside 10, which is, however, reacted not with n-butylamine but with morpholine, compound 41 is obtained as a pale yellow wax. C₂₇H₃₈FN₃O₇ (535.6): MS (ESI⁺) 536.48 (M+H⁺).

### Compound 42

### Example 30

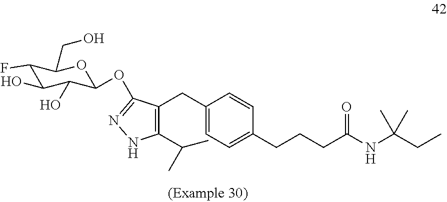

Compound 42 is synthesized in analogy to the synthesis route described for compound 13 (example 2). Starting from the glycoside 10, which is, however, reacted not with n-butylamine but with tert-amylamine, compound 42 is obtained as a pale yellow wax. C₂₈H₄₂FN₃O₆ (535.7): MS (ESI⁺) 536.54 (M+H⁺).

### Compound 43

### Example 31

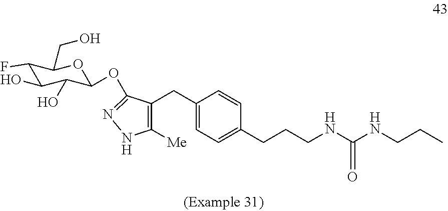

41.3 mg of 1-allyl-3-propylurea are dissolved in 5.00 ml of THF, and 1.21 ml of a 0.5M 9-BBN solution in toluene are added, and the mixture is stirred at 20° C. for 4 h. Subsequently, a solution of 180 mg of the glycoside 17 in 10.0 ml of toluene, 7.4 mg of tri-o-tolylphosphine, 102.7 mg of potassium phosphate and 2.7 mg of Pd(OAc)₂ are added. The reaction mixture is heated at 100° C. for 3 h. The precipitate is filtered off, and the organic phase is washed with 10 ml of water and dried over magnesium sulfate. The solvent is removed in a rotary evaporator, and the crude product is purified by chromatography on silica gel (EtOAc/heptane). 59 mg of a colorless solid are obtained and reacted with sodium methoxide in analogy to the preparation of compound 13 (example 2). Compound 43 is obtained as a colorless wax. C₂₄H₃₅FN₄O₆ (494.6) MS (ESI⁺) 494.12 (M⁺).

### 4-(2-Ethoxycarbonyl-4-methyl-3-oxo-pent-1-enyl)benzoic acid (E/Z isomer mixture) (44)

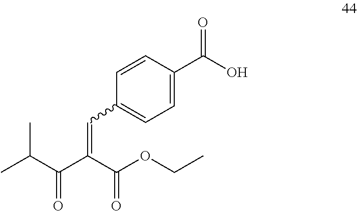

29.0 g of ethyl isobutyrylacetate and 33.0 g of 4-carboxybenzaldehyde are heated with a water trap for 6 h. The reaction solution is concentrated, taken up in ethyl acetate and extracted with 20% strength ammonium chloride solution and saturated sodium chloride solution. The organic phase is dried over magnesium sulfate, concentrated and directly reacted further to 45. 50.0 g of an oil are obtained. C₁₆H₁₈O₅ (290.3): MS (ESI⁺): 291.1 (M+H)⁺, t_{R}=1.42 min (Gradient 2).

### 4-(2-Ethoxycarbonyl-4-methyl-3-oxo-pentyl)benzoic acid (45)

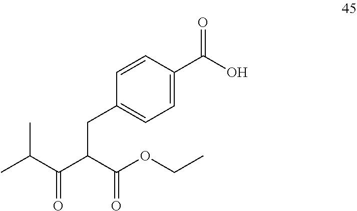

50 g of 4-(2-ethoxycarbonyl-4-methyl-3-oxo-pent-1-enyl)benzoic acid are dissolved in 300 ml of THF, 1.00 g of palladium on carbon (10%) is added, and the mixture is hydrogenated under a hydrogen pressure of 4 bar in an autoclave for 24 h. The mixture is diluted with dichloromethane and filtered with suction through Celite, the residue is washed with dichloromethane and concentrated in vacuo. The residue is purified by chromatography on silica gel (ethyl acetate/n-heptane=3/1). 45 g of compound 45 are obtained as an oil. C₁₆H₂₀O₅ (292.3) MS (ESI⁺): 293.1 (M+H)⁺, t_{R}=1.37 min (Gradient 2).

### 4-[1-(2-Cyanoethyl)-5-hydroxy-3-isopropyl-1H-pyrazol-4-ylmethyl]benzoic acid (46)

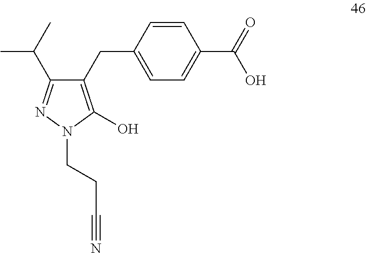

15 g of compound 45 are dissolved in 100 ml of glacial acetic acid. 7.4 ml of 2-cyanoethylhydrazine are added, and the solution is heated at 100° C. for 2 h. The mixture is added to ice-water and extracted several times with ethyl acetate. The organic phase is extracted with 20% strength ammonium chloride solution and saturated sodium chloride solution and dried over sodium sulfate. 2.40 g of the desired compound 46 crystallize out with the ethyl acetate phase. The mother liquor is concentrated and chromatographed on silica gel (dichloromethane:methanol:glacial acetic acid=100:10:1). A further 1.10 g of compound 46, plus 7.0 g of reisolated precursor 45, are obtained. C₁₇H₁₉N₃O₃ (313.4); MS (ESI⁺): 314.2 (M+H)⁺, t_{R}=0.97 min (Gradient 2).

### N-(2-Carbamoylethyl)-4-[1-(2-cyano-ethyl)-5-hydroxy-3-isopropyl-1H-pyrazol-4-ylmethyl]benzamide (47)

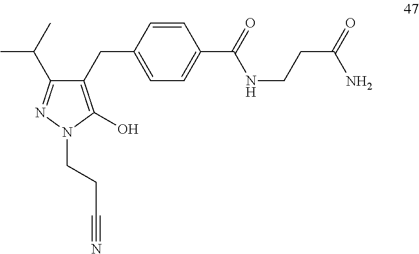

500 mg of compound 46 and 145 mg of -alaninamide hydrochloride are introduced into 10 ml of dichloromethane, and 0.8 ml of N,N-diisopropylethylamine, 215 mg of 1-hydroxybenzotriazole and 306 mg of 1-ethyl-3-(3-dimethylaminopropyl)carbodiimide hydrochloride are added. The solution is stirred for 12 h. The solution is concentrated and the crude product is purified by chromatography on silica gel (dichloromethane/methanol/glacial acetic acid 100:0:5→100:10:5). 440 mg of the desired compound 47 are obtained. C₂₀H₂₅N₅O₃ (383.5); MS (ESI⁺): 384.2 (M+H)⁺, t_{R}=3.58 min (Gradient 3).

### Compound 48

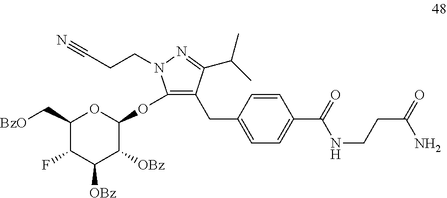

300 mg of compound 47, 436 mg of compound 4, and 324 mg of potassium carbonate are suspended in 25 ml of acetonitrile and 2.5 ml of water and stirred for 72 h. The reaction mixture is filtered, the residue is washed with dichloromethane, and the combined organic phase is extracted with water and saturated sodium chloride solution. The organic phase is dried over sodium sulfate and the residue is chromatographed on silica gel (dichloromethane/methanol=100/5). 207 mg of the glycoside 48 are obtained as a colorless solid. C₄₇H₄₆FN₅O₁₀ (859.9); MS (ESI⁺): 860.3 (M+H)⁺, t_{R}=1.70 min (Gradient 2).

### Compound 49

### Example 32

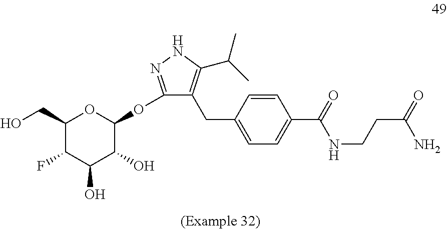

200 mg of compound 48 are dissolved in 15 ml of THF and cooled to −78° C. under argon. 0.81 ml of lithium bis(trimethylsilyl)amide solution (1M in hexane) is slowly added through a septum. After 30 min, 2 ml of 20% strength ammonium chloride solution are added in the cold, and the solution is warmed to room temperature. 2 ml of saturated sodium chloride solution are added, the organic phase is separated off, and the aqueous phase is extracted twice with ethyl acetate. The combined organic phase is concentrated and the residue is taken up in a mixture of triethylamine:methanol:water (14 ml 1:3:3). The solution is stirred for 24 h and is then concentrated to dryness and purified by chromatography on silica gel. 50 mg of compound 49 are obtained as a colorless solid. C₂₃H₃₁FN₄O₇ (494.5); MS (ESI⁻): 493.2 (M−H)⁻, t_{R}=3.58 min (Gradient 3); t_{R}=0.97 min (Gradient 1).

### Compound 50

### Example 33

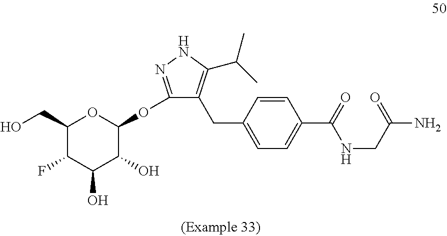

Compound 50 is synthesized in analogy to the synthesis described for compound 49 (example 32). However, glycinamide hydrochloride is employed instead of β-alaninamide. Compound 50 is obtained as a colorless solid. C₂₂H₂₉FN₄O₇ (480.5): MS (ESI⁺) 481.19 (M+H⁺).

### Compound 51

### Example 34

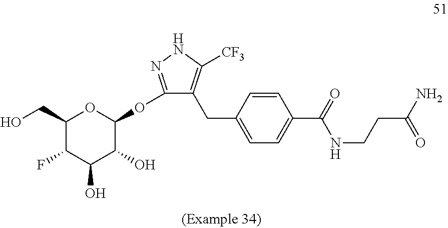

Compound 51 is synthesized in analogy to the synthesis described for compound 49 (example 32). However, 4,4,4-trifluoroacetoacetate is used as starting material instead of ethyl isobutylacetate. Compound 51 is obtained as a colorless solid. C₂₁H₂₄F₄N₄O₇ (520.4): MS (ESI⁺) 521.16 (M+H⁺).

### Compound 52

### Example 35

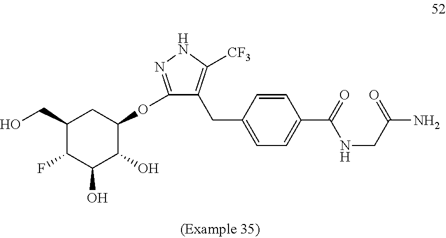

Compound 52 is synthesized in analogy to the synthesis described for compound 50 (Example 33). However, 4,4,4-trifluoroacetoacetate is used as starting material instead of ethyl isobutylacetate. Compound 52 is obtained as a colorless solid. C₂₀H₂₂F₄N₄O₇ (506.4): MS (ESI⁺) 507.16 (M+H⁺).

### Compound 53

### Example 36

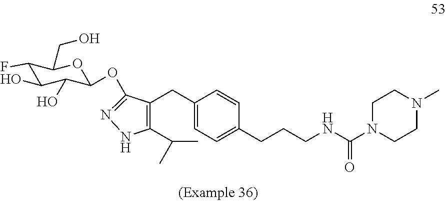

Compound 53 is synthesized in analogy to the synthesis described for compound 43 (example 31), but 1-(N-methylpiperazine)-3-allylurea is used as starting material instead of 1-allyl-3-propylurea, and the glycoside 7 is employed instead of the glycoside 17. Compound 53 is obtained as a colorless solid. C₂₈H₄₂FN₅O₆ (563.7).

### Compound 54

### Example 37

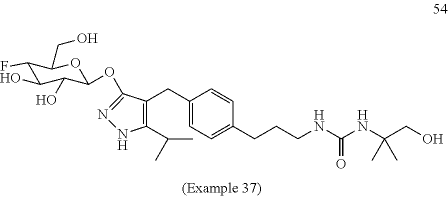

Compound 54 is synthesized in analogy to the synthesis described for compound 43 (example 31), but 1-(N-methylpiperazine)-3-allylurea is used as starting material instead of 1-allyl-3-propylurea, and the glycoside 7 is employed instead of the glycoside 17. Compound 54 is obtained as a colorless solid. C₂₇H₄₁FN₄O₇ (552.7).

### Experimental Part

### Compounds of Formula II

### Reaction Scheme

### Synthesis of α-Bromoglycosides

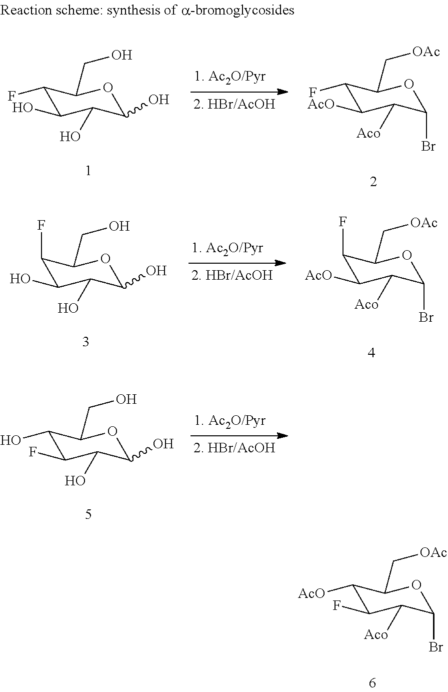

### 1-Bromo-4-deoxy-4-fluoro-2,3,6-tri-O-acetyl-alpha-D-glucose (2)

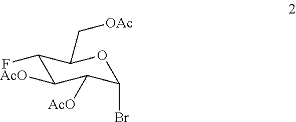

5.0 g (27.5 mmol) of 4-deoxy-4-fluoro-D-glucopyranose 1 (Apollo) are suspended in 50 ml of pyridine and 50 ml of acetic anhydride. The reaction solution is stirred at 45° C. for 4 hours. This results in a clear reaction solution which is concentrated. 12.0 g of crude product are obtained. This crude product is dissolved in 160 ml of 33% strength HBr in glacial acetic acid and left to stand at room temperature for 2 hours. The reaction solution is then poured into a mixture of 300 g of ice and 300 ml of ethyl acetate. The organic phase is washed twice with aqueous NaCl solution, filtered through a little silica gel and concentrated. The residue is separated by chromatography on silica gel (ethyl acetate/heptane=1/1). 8.19 g (80% over 2 stages) of 2 are obtained as a pale yellow solid.

### 1-Bromo-4-deoxy-4-fluoro-2,3,6-tri-O-acetyl-alpha-D-galactose (4)

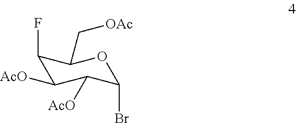

100 mg (0.55 mmol) of 3 are reacted with 3.5 ml of pyridine and 3.5 ml of acetic anhydride in analogy to the preparation of compound 2. 89 mg (44%) of 4 are obtained as an amorphous solid.

### 1-Bromo-3-deoxy-3-fluoro-2,4,6-tri-O-acetyl-alpha-D-glucose (6)

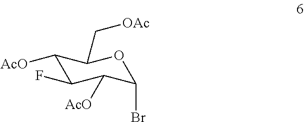

335 mg (1.84 mmol) of 5 are reacted with 10 ml of pyridine and 10 ml of acetic anhydride in analogy to the preparation of compound 2. 628 mg (92%) of 6 are obtained as an amorphous solid.

### Reaction Scheme

### Synthesis of the α-Bromoglycoside 10

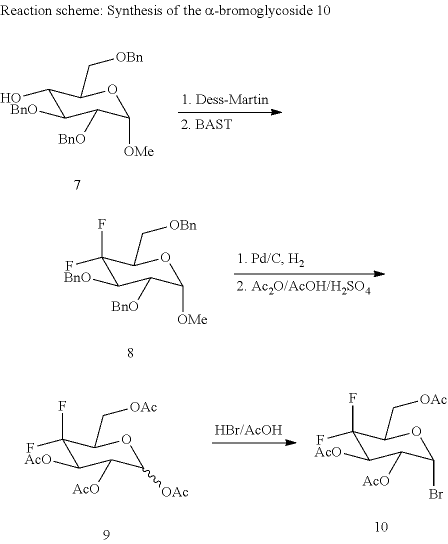

### 1-Methoxy-4-deoxy-4,4-difluoro-2,3,6-tri-O-benzyl-alpha-D-glucose (8)

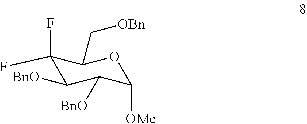

3.69 g (7.9 mmol) of 1-methoxy-2,3,6-tri-O-benzyl-alpha-D-glucose 7 (Tetrahedron Asymmetry 2000, 11, 385-387) were dissolved in 110 ml of methylene chloride and, under an argon atmosphere, 3.6 g (8.5 mmol) of Dess-Martin reagent (Aldrich) are added dropwise. After 3 hours at room temperature, the mixture is diluted with 300 ml of ethyl acetate/n-heptane (1:1) and washed 1× with NaHCO₃ and 1× with Na₂S₂O₃ solution. The organic phase is filtered through silica gel and concentrated. The residue is separated by chromatography on silica gel (ethyl acetate/n-heptane 1:1). 2.90 g (79%) of the ketone are obtained. This is dissolved in 30 ml of methylene chloride and, under an argon atmosphere, 4.0 ml of BAST ([bis(2-methoxyethyl)amino]sulfur trifluoride, Aldrich) are added dropwise. After 20 hours at room temperature, the mixture is diluted with 200 ml of ethyl acetate and washed carefully (extensive effervescence) with cold NaHCO₃ solution. The organic phase is filtered through silica gel and concentrated. The residue is separated by chromatography on silica gel (ethyl acetate/n-heptane 1:1). 2.6 g (85%) of 8 are obtained as a colorless oil.

### 4-Deoxy-4,4-difluoro-1,2,3,6-tetra-O-acetyl-alpha-D-glucose (9)

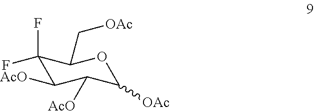

2.30 g (4.7 mmol) of 8 and 2.0 g of Pd/C (10% Pd) are dissolved in 150 ml of methanol and 10 ml of acetic acid and hydrogenated under an atmosphere of 5 bar of hydrogen at room temperature for 16 h. The reaction solution is concentrated and the residue is purified by flash chromatography (methylene chloride/methanol/conc. ammonia, 30/5/1). Yield 850 mg (83%) of 1-methoxy-4-deoxy-4,4-difluoro-alpha-D-glucose as white amorphous solid. C₇H₁₂F₂O₅ (214.17) MS (DCI): 215.4 (M+H⁺). 700 mg (3.3 mmol) of this are dissolved in 3.5 ml of acetic acid and 6.3 ml of acetic anhydride. Addition of 0.2 ml of conc. H₂SO₄ is followed by stirring at 60° C. for 5 h. The reaction solution is then poured into a mixture of 30 g of ice and 30 ml of ethyl acetate. The organic phase is washed twice with aqueous NaCl solution, filtered through a little silica gel and concentrated. The residue is separated by chromatography on silica gel (ethyl acetate/n-heptane 1:1). 300 mg (25%) of 9 are obtained as a mixture of anomers. C₁₄H₁₈F₂O₉ (368.29) MS (DCI): 369.3 (M+H⁺)

### 1-Bromo-4-deoxy-4,4-difluoro-2,3,6-tri-O-acetyl-alpha-D-glucose (10)

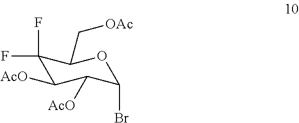

300 mg (0.8 mmol) of tetraacetate 9 are dissolved in 13 ml of 33% strength HBr in glacial acetic acid and left to stand at room temperature for 6 hours. The reaction solution is then poured into a mixture of 10 g of ice and 10 ml of ethyl acetate. The organic phase is washed twice with aqueous NaCl solution, filtered through a little silica gel and concentrated. The residue is separated by chromatography (SiO₂) (ethyl acetate/heptane 1:1). 112 mg (35%) of 10 are obtained as a colorless solid. C₁₂H₁₅BrF₂O₇ (389.15) MS (DCI): 389.2 (M+H⁺).

### Reaction Scheme

### Synthesis of the α-Bromoglycosides 14

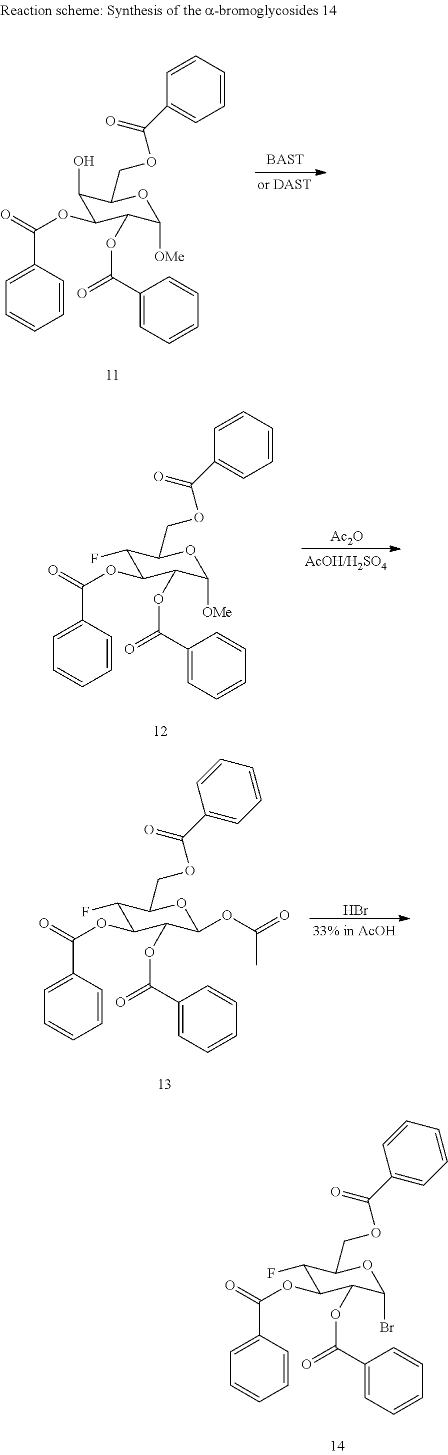

### Methyl 2,3,6-tri-O-benzoyl-4-fluoro-4-deoxy-α-D-glucopyranoside (12)

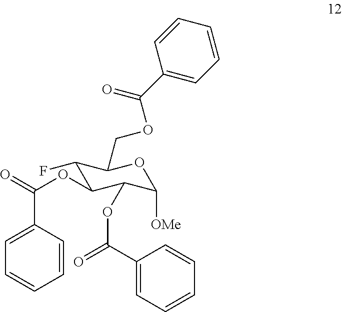

3.0 g of methyl 2,3,6-tri-O-benzoyl-α-D-galactopyranoside (Reist et al., J. Org. Chem. 1965, 30, 2312) are introduced into dichloromethane and cooled to −30° C. Then 3.06 ml of [bis(2-methoxyethyl)amino]sulfur trifluoride (BAST) are added dropwise. The reaction solution is warmed to room temperature and stirred for 12 h. The mixture is diluted with dichloromethane, and the organic phase is extracted with H₂O, NaHCO₃ solution and saturated NaCl solution. The organic phase is dried over Na₂SO₄ and concentrated. The crude product is crystallized from ethyl acetate and heptane. 1.95 g of the product 12 are obtained as a colorless solid. C₂₈H₂₅FO₈ (508.51) MS (ESI⁺) 526.18 (M+NH₄⁺). Alternatively, the reaction can also be carried out using 2.8 eq. of diethylaminosulfur trifluoride (DAST); in this case, the reaction solution is refluxed for 18 h after addition. Working up takes place in analogy to the above description.

### 1-O-Acetyl-2,3,6-tri-O-benzoyl-4-fluoro-4-deoxy-glucose (13)

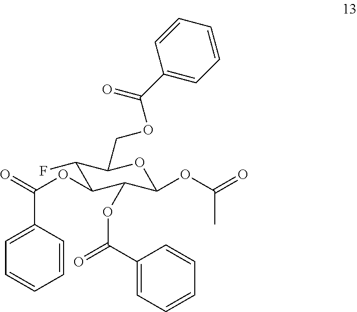

12.0 g of the compound methyl 2,3,6-tri-O-benzoyl-4-fluoro-4-deoxy-α-D-glucopyranoside are suspended in 150 ml of acetic anhydride. 8.4 ml of conc. sulfuric acid are mixed with 150 ml of glacial acetic acid and added to the mixture while cooling in ice. The mixture is stirred at room temperature for 60 h. The reaction mixture is poured into NaHCO₃ solution, and this solution is extracted with chloromethane. The organic phase is washed with NaCl solution, dried with Na₂SO₄ and concentrated. The residue is recrystallized from ethyl acetate and heptane. 5.97 g of the product 13 are obtained as a colorless solid.

C₂₉H₂₅FO₉ (536.52) MS (ESI⁺) 554.15 (M+NH₄⁺).

### 1-Bromo-4-deoxy-4-fluoro-2,3,6-tri-O-benzoyl-alpha-D-glucose (14)

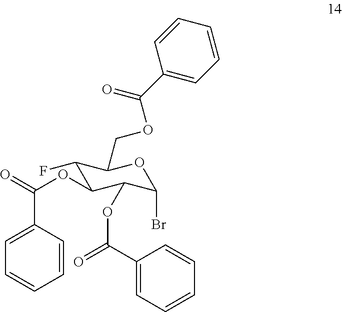

1.44 g of 1-O-acetyl, 2,3,6-tri-O-benzoyl-4-fluoro-4-deoxyglucose are dissolved in 20 ml of hydrobromic acid in glacial acetic acid (33%) and stirred at room temperature. After 5 hours, the mixture is added to ice-water, and the aqueous phase is extracted three times with dichloromethane. The collected organic phase is washed with saturated sodium chloride solution, dried over sodium sulfate and evaporated to dryness. The crude product is filtered with ethyl acetate/heptane (70:30) through silica gel. 1.40 g of the product 14 are obtained as a colorless solid. C₂₇H₂₂BrFC₇ (557.37) MS (ESI⁺) 574.05/576.05 (M+NH₄⁺).

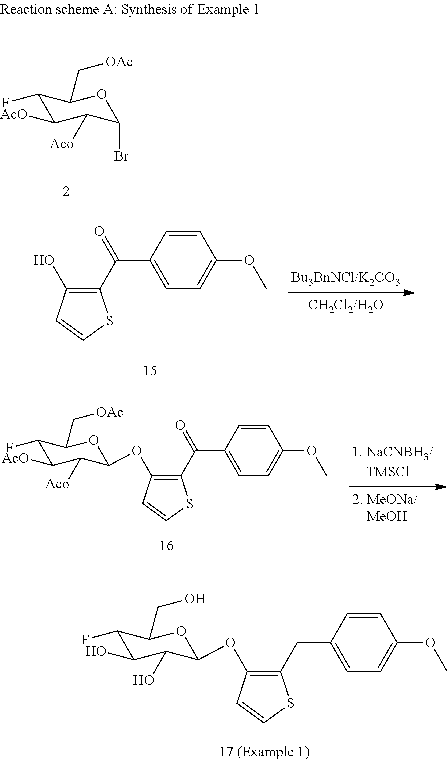

Further Exemplary Compounds:

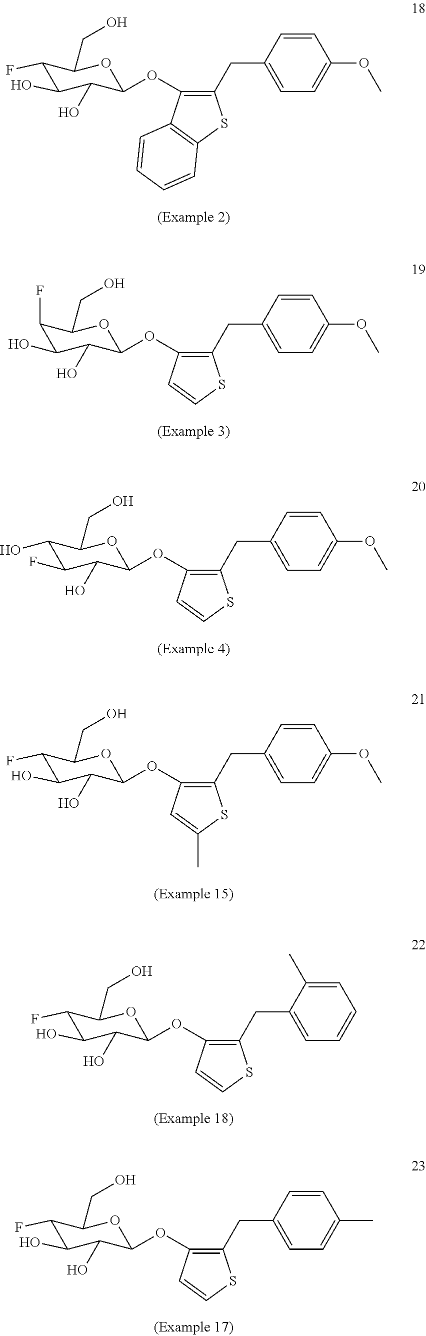

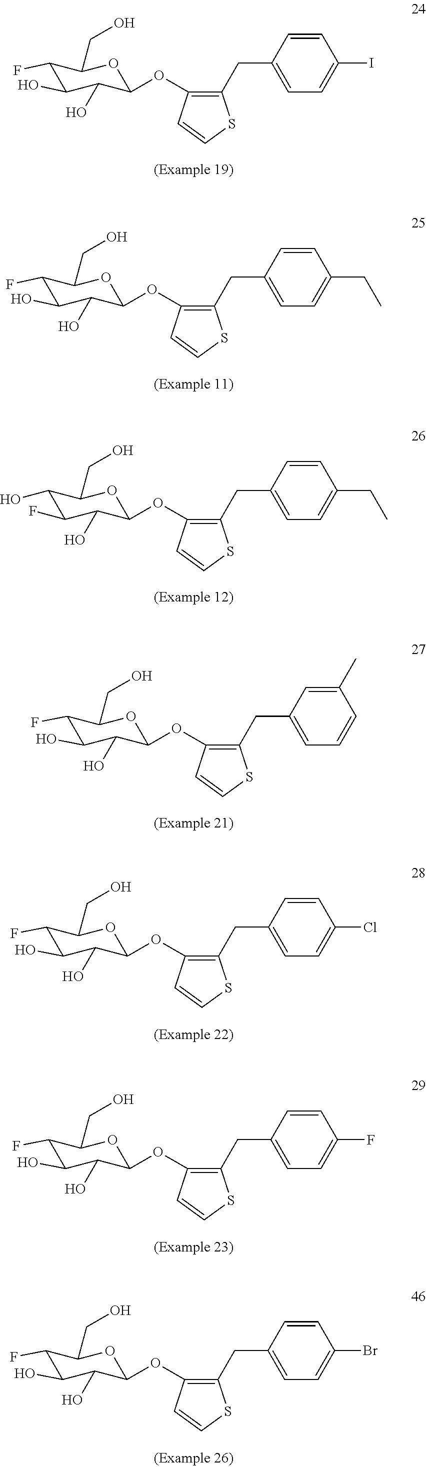

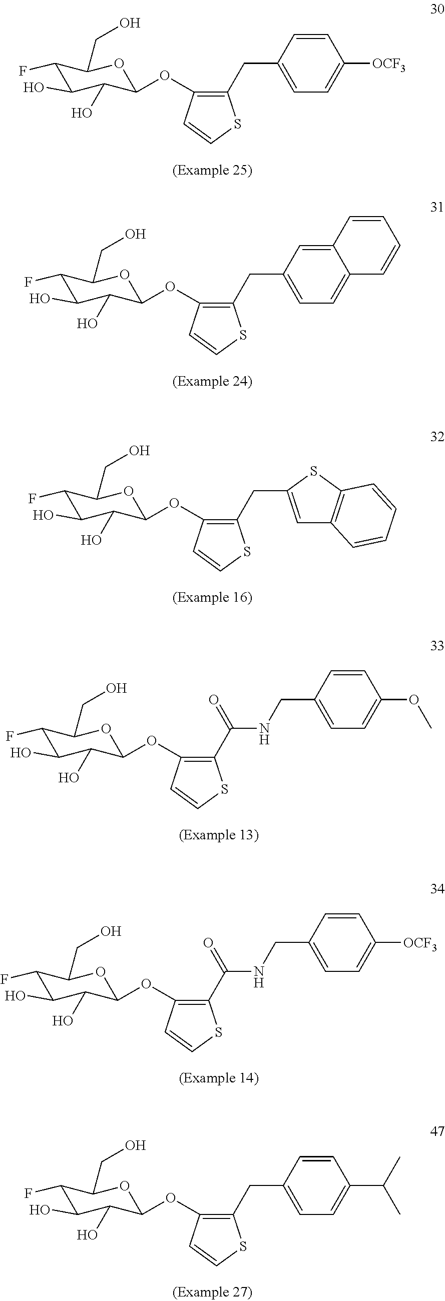

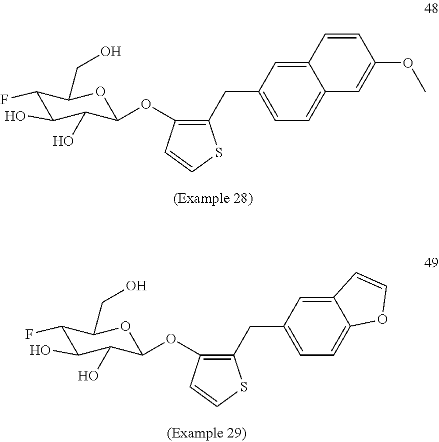

### Example 1

### Compound 17

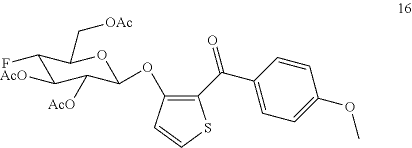

400 mg (1.7 mmol) of (3-hydroxythiophen-2-yl)(4-methoxyphenyl)methanone (15) CDE Application Number 10231370.9 (2002/0049) and 200 mg (0.54 mmol) of bromide 2 are dissolved in 6 ml of methylene chloride. 160 mg of Bu₃BnNCl (PTC=phase transfer catalyst), 320 mg of K₂CO₃ and 0.4 ml of water are successively added to this solution, which is then stirred at room temperature for 20 hours. The reaction solution is diluted with 20 ml of ethyl acetate and filtered through silica gel. The filtrate is concentrated and the residue is separated by chromatography over silica gel (ethyl acetate/heptane=1/1). 160 mg (56%) of 16 are obtained as a colorless solid. C₂₄H₂₅FO₁₀S (524.52) MS (ESI⁺) 525.12 (M+H⁺).

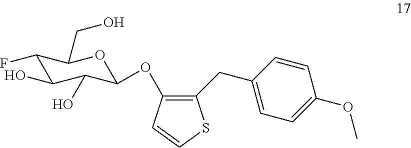

150 mg (0.29 mmol) of compound 16 are dissolved in 4 ml of acetonitrile. This solution is cooled in an ice bath and then 150 mg of NaCNBH₃ and 0.2 ml of TMSCl are added. The cooling is then removed and the mixture is stirred at room temperature for 2 hours. The reaction solution is diluted with 20 ml of ethyl acetate and filtered through silica gel. The filtrate is concentrated, and 150 mg of crude product are obtained. This crude product is taken up in 4 ml of methanol, and 1 ml of 1M NaOMe in MeOH is added. After one hour, the mixture is neutralized with methanolic HCl and concentrated, and the residue is purified by chromatography on silica gel (methylene chloride/methanol/conc. ammonia, 30/5/1). 76 mg (69% over 2 stages) of 17 are obtained as a colorless solid. C₁₈H₂₁FO₆S (384.43) ME (ESI⁺) 403.21 (M+H₂O+H⁺).

### Example 2

### Compound 18

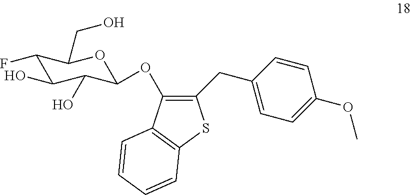

100 mg (0.47 mmol) of (3-hydroxybenzothiophene-2-yl)(4-methoxyphenyl)methanone (Eur. J. Med. Chem. 1985, 20, 187-189) and 300 mg (0.80 mmol) of bromide 2 are dissolved in 10 ml of chloroform. 120 mg of Bu₃BnNCl (PTC=phase-transfer catalyst) and 1.5 ml of 1 N aqueous sodium hydroxide solution are successively added to this solution, which is then boiled under reflux for 4 hours. The reaction solution is diluted with 20 ml of ethyl acetate and filtered through silica gel. The filtrate is concentrated and the residue is separated by chromatography on silica gel (ethyl acetate/heptane=1/1). 135 mg (51%) of pale yellow solid are obtained. This is converted into compound 18 with 100 mg of NaCNBH₃ and 0.2 ml of TMSCl and then with NaOMe/MeOH in analogy to the preparation of compound 17. 46 mg of 18 are obtained. C₂₂H₂₃FO₆S (434.49) MS (ESI⁻) 479.18 (M+CHO₂⁻).

### Example 3

### Compound 19

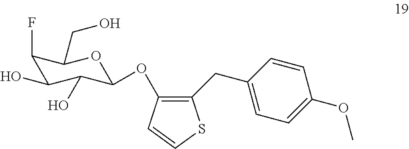

178 mg of (3-hydroxythiophen-2-yl)(4-methoxyphenyl)methanone (15) and 90 mg of bromide 4 are reacted in analogy to the synthesis of example 1, and 49 mg of 19 are obtained as a colorless solid. C₁₈H₂₁FO₆S (384.43) MS (ESI⁺) 403.21 (M+H₂O+H⁺).

### Example 4

### Compound 20

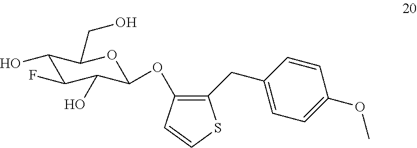

200 mg of (3-hydroxythiophen-2-yl)(4-methoxyphenyl)methanone 15 and 100 mg of bromide 6 are reacted in analogy to the synthesis of example 1, and 59 mg of 20 are obtained as a colorless solid. C₁₈H₂₁FO₆S (384.43) MS (ESI⁺) 403.21 (M+H₂O+H⁺).

Examples 11 (compound 25) and 15 (compound 21) are synthesized in analogy to the synthesis of example 1 starting from the appropriate hydroxythiophenes and the bromide 2.

Examples 16 (compound 32), 17 (compound 23), 18 (compound 22), 19 (compound 24), 21 (compound 27), 22 (compound 28), 23 (compound 29), 24 (compound 31), 25 (compound 30), 26 (compound 46), 27 (compound 47), 28 (compound 48) and 29 (compound 49) are synthesized in analogy to the synthesis of example 1 starting from appropriate hydroxythiophenes and the bromide 14.

Example 12 Compound 26 is synthesized in analogy to the synthesis of example 4 starting from the appropriate hydroxythiophene and bromide 6.

Examples 13 (compound 33) and 14 (compound 34) are synthesized in analogy to the synthesis of compound 16 by reacting the appropriate hydroxythiophenes with the bromide 2 and subsequently deprotecting with NaOMe/MeOH in analogy to example 1.

Example 20 Compound 35 is synthesized in analogy to the synthesis of example 1 starting from hydroxythiophene 15 and the bromide 10.

### Reaction Scheme B

### Synthesis of Example 5

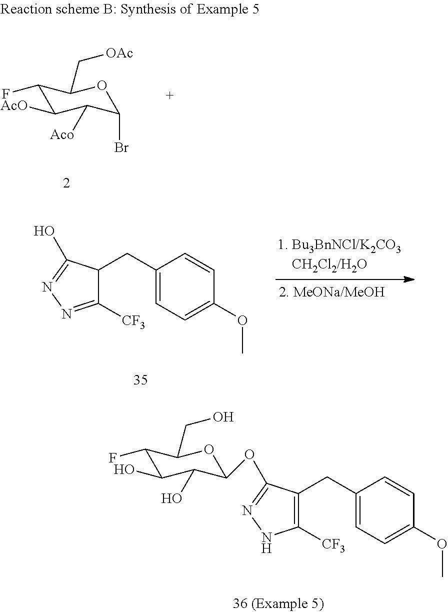

Further Exemplary Compounds:

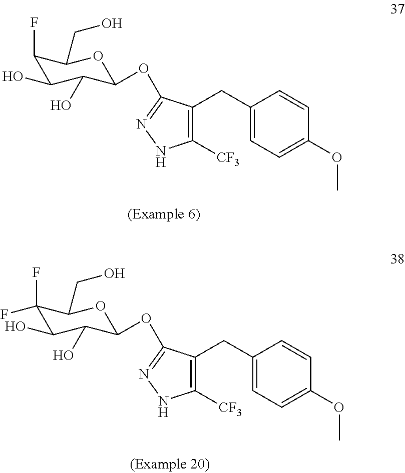

### Example 5

### Compound 36

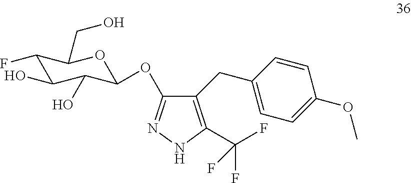

200 mg of 4-(4-methoxybenzyl)-5-methyl-1H-pyrazol-3-ol (35) (J. Med. Chem. 1996, 39, 3920-3928) are glycosilated with 100 mg of bromide 2 in analogy to the synthesis of example 1 and then deprotected with NaOMe/MeOH in analogy to example 1. 49 mg of compound 36 are obtained as a colorless solid. C₁₈H₂₀F₄N₂O₆ (436.36) MS (ESI⁺) 437.21 (M+H⁺).

### Example 6

### Compound 37

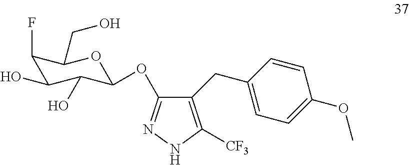

200 mg of 4-(4-methoxybenzyl)-5-methyl-1H-pyrazol-3-ol (35) and 100 mg of bromide 4 are glycosilated in analogy to the synthesis of example 1 and then deprotected with NaOMe/MeOH in analogy to example 1. 89 mg of compound 37 are obtained as a colorless solid. C₁₈H₂₀F₄N₂O₆ (436.36) MS (ESI⁺) 437.21 (M+H⁺).

### Example 20

### Compound 38

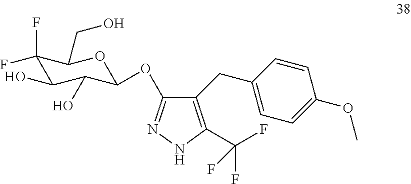

110 mg of 4-(4-methoxybenzyl)-5-methyl-1H-pyrazol-3-ol (35) and 60 mg of bromide 10 are glycosilated in analogy to the synthesis of example 1 and then deprotected with NaOMe/MeOH in analogy to example 1. 49 mg of the compound 38 are obtained as a colorless solid. C₁₈H₁₉F₅N₂O₆ (454.35) MS (ESI⁺) 455.22 (M+H⁺).

### Reaction Scheme C

### Synthesis of Example 8 and Example 10

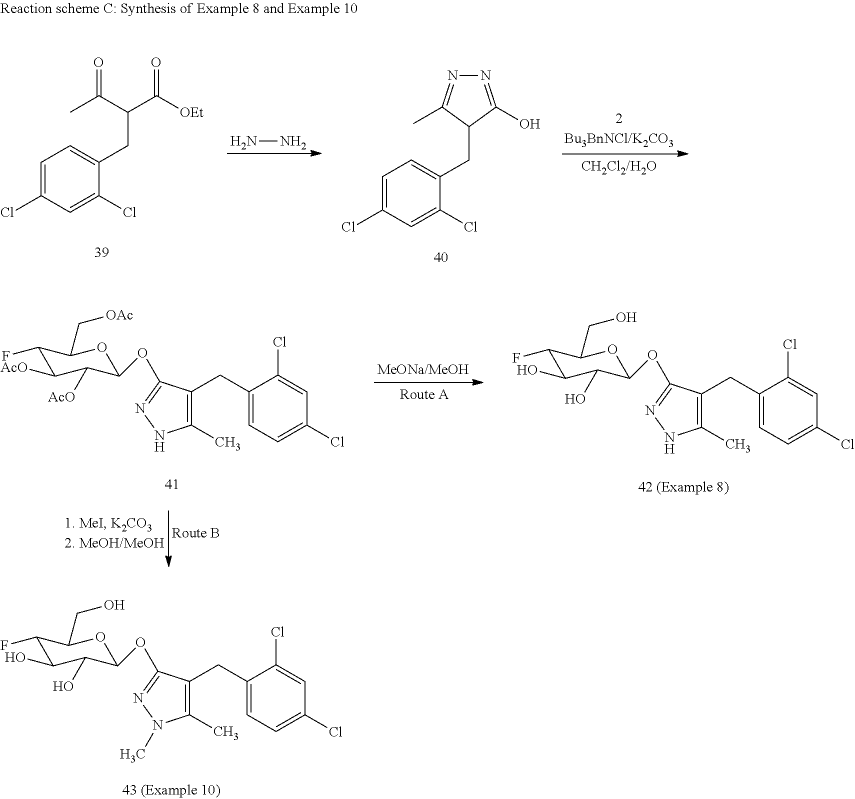

Further Exemplary Compounds:

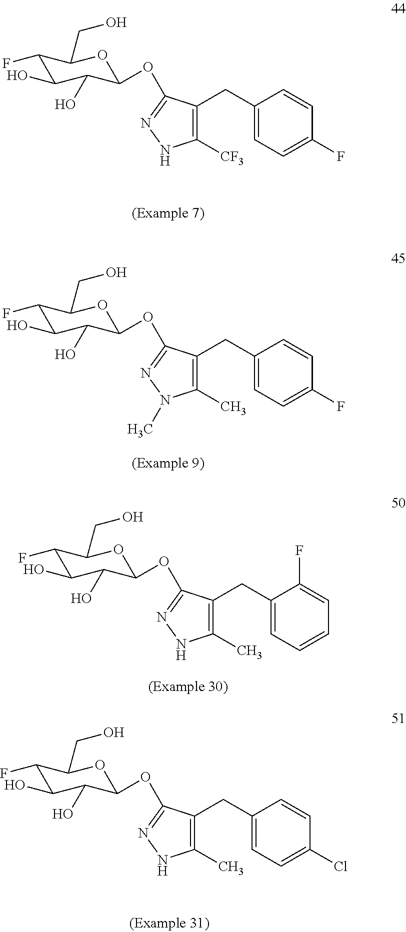

### Example 8

### Compound 42

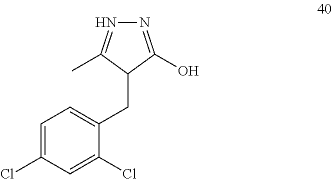

500 mg (1.73 mmol) of ethyl 2-(2,4-dichlorobenzyl)-3-oxobutyrate (39) (Bionet) are boiled with 0.21 ml of 51% pure hydrazine hydrate (3.46 mmol) in 15 ml of toluene with a water trap for 1.5 h. After cooling, the solid is filtered off with suction and washed with toluene and ether. 400 mg (90%) of the compound 40 are obtained as a voluminous white precipitate. C₁₁H₁₀C₁₂N₂O (257.12) MS (ESI): 257 (M+H⁺).

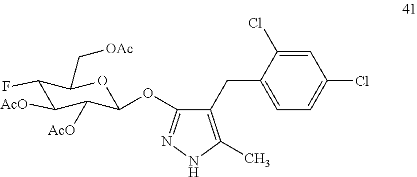

270 mg (1.05 mmol) of 4-(2,4-dichlorobenzyl)-5-methyl-1H-pyrazol-3-ol (40) were dissolved in 25 ml of methylene chloride, and 0.7 ml of water, 1.2 g (8.68 mmol) of potassium carbonate, 84 mg (0.31 mmol) of benzyltriethylammonium bromide and 428 mg (1.15 mmol) of bromide 2 were added, and the mixture was stirred at RT for 18 h. The reaction solution was diluted with methylene chloride and washed once each with water and saturated brine, dried over MgSO₄ and concentrated. The crude product was purified on silica gel. 122 mg (21%) of the compound 41 are obtained as white solid. C₂₃H₂₅Cl₂FN₂O₈ (547.37) MS (ESI): 547 (M+H⁺).

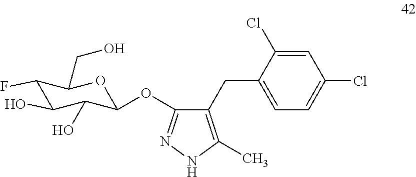

70 mg of (0.1278 mmol) of the compound 41 are dissolved in accordance with route A in 2 ml of methanol, and 1.02 ml (0.511 mmol) of sodium methanolate solution (0.5M) in tetrahydrofuran are added. After 5 min,

27.6 mg (0.516 mmol) of ammonium chloride and 2.0 g of SiO₂ are added. The solution is concentrated and the product is filtered through silica gel and washed first with EtOAc and then with EtOAc/methanol 20:1. 50 mg (90%) of the compound 42 are obtained as a colorless solid.

C₁₇H₁₉C₁₂FN₂O₅ (421.26) MS (ESI): 420 (M+H⁺).

### Example 10

### Compound 43

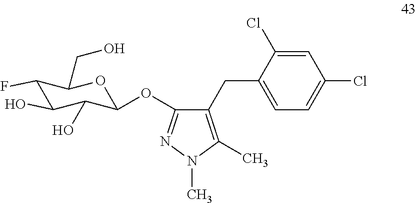

50 mg of compound 41 are dissolved in accordance with route B in 2.0 ml of DMF and, at room temperature, 50 mg of K₂CO₃ and 57 μl of methyl iodide are added. After 14 days, 30 ml of EtOAc are added, and the organic phase is washed twice with 20 ml of H₂O each time and concentrated. The crude product is purified by column chromatography (EtOAc/heptane=3:1) and reacted with NaOMe/MeOh in analogy to the preparation of compound 42. 9.1 mg of compound 43 are obtained as a colorless wax. C₁₈H₂₁C₁₂FN₂O₅ (435.24) MS (ESI): 434 (M+H⁺).

Examples 7 (compound 44), 30 (compound 50) and 31 (compound 51) are synthesized in analogy to the synthesis described for example 8 (compound 42) starting from the appropriate β-keto esters.

Example 9 Compound 45 is synthesized in analogy to the synthesis described for example 10 (compound 43) starting from the appropriate β-keto ester.

### Experimental Part

### Compounds of Formula III

### Reaction Scheme

### Synthesis of -Bromoglycosides

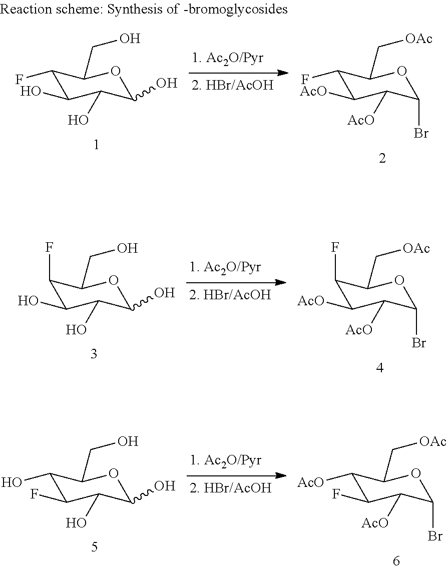

### 1-Bromo-4-deoxy-4-fluoro-2,3,6-tri-O-acetyl-alpha-D-glucose 2

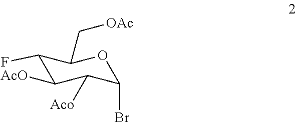

5 g (27.5 mmol) of 4-deoxy-4-fluoro-D-glucopyranose 1 (Apollo) are suspended in 50 ml of pyridine and 50 ml of acetic anhydride. The reaction solution is stirred at 45° C. for 4 hours. This results in a clear reaction solution which is then concentrated. 12 g of crude product are obtained. This crude product is dissolved in 160 ml of 33% strength HBr in glacial acetic acid and left to stand at room temperature for 2 hours. The reaction solution is then poured into a mixture of 300 g of ice and 300 ml of ethyl acetate. The organic phase is washed twice more with aqueous NaCl solution, filtered through a little silica gel and concentrated. The residue is separated by chromatography on silica gel (ethyl acetate/heptane=1/1). 8.19 g (80% over 2 stages) of 2 are obtained as a pale yellow solid.

### 1-Bromo-4-deoxy-4-fluoro-2,3,6-tri-O-acetyl-alpha-D-galactose 4

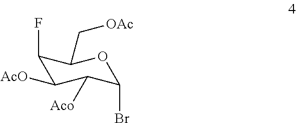

100 mg (0.55 mmol) of 3 are reacted with 3.5 ml of pyridine and 3.5 ml of acetic anhydride in analogy to the preparation of compound 2. 89 mg (44%) of 4 are obtained as an amorphous solid.

### 1-Bromo-3-deoxy-3-fluoro-2,4,6-tri-O-acetyl-alpha-D-glucose 6

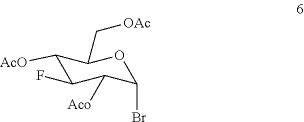

335 mg (1.84 mmol) of 5 are reacted with 10 ml of pyridine and 10 ml of acetic anhydride in analogy to the preparation of compound 2. 628 mg (92%) of 6 are obtained as an amorphous solid.

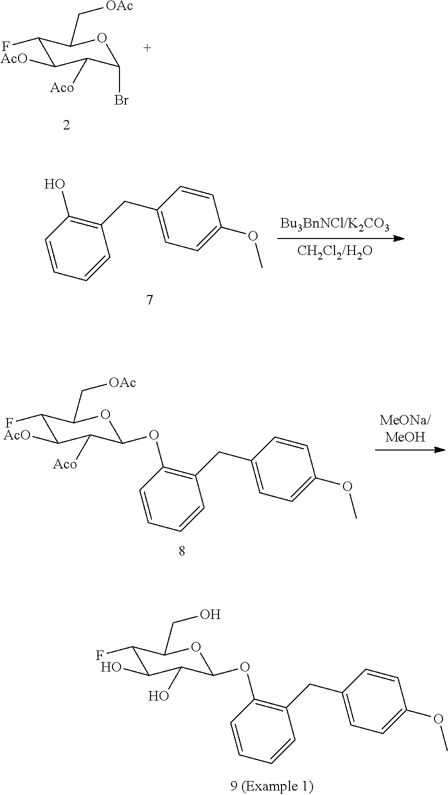

The following were prepared in an analogous manner:

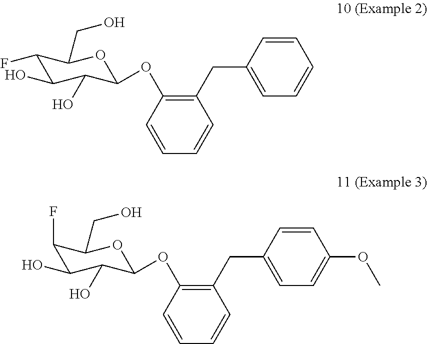

### Example 1

### Compound 9

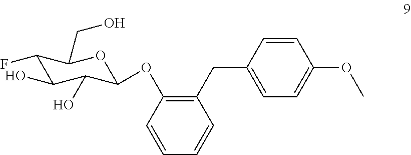

100 mg (0.47 mmol) of 2-(4-methoxybenzyl)phenol 7 and 370 mg (1.17 mmol) of bromide 2 are dissolved in 6 ml of methylene chloride. 160 mg of Bu₃BnNCl (PTC=phase transfer catalyst), 320 mg of K₂CO₃ and 0.4 ml of water are successively added to this solution, which is then stirred at room temperature for 20 hours. The reaction solution is diluted with 20 ml of ethyl acetate and filtered through silica gel. The filtrate is concentrated and the residue is separated by chromatography on silica gel (ethyl acetate/heptane=1/1). 72 mg of 8 are obtained as a colorless solid. The resulting 72 mg of 8 are taken up in 4 ml of methanol, and 1 ml of 1N NaOMe/MeOH is added. After one hour, the mixture is neutralized with methanolic HCl and concentrated, and the residue is separated by chromatography on silica gel (methylene chloride/methanol/conc. ammonia, 30/5/1). 29 mg of 9 are obtained as a colorless solid. C₂₀H₂₃FO₆ (378.40) MS (ESI⁻) 423.22 (M+CHO₂⁻).

### Example 2

### Compound 10

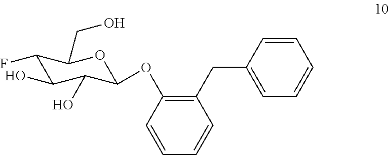

100 mg (0.47 mmol) of 2-benzylphenol and 370 mg (1.17 mmol) of bromide 2 are reacted in analogy to the synthesis of compound 9, and 31 mg of 10 are obtained as a colorless solid. C₁₉H₂₁FO₅ (348.37) MS (ESI⁻) 393.15 (M+CHO₂⁻).

### Example 3

### Compound 11

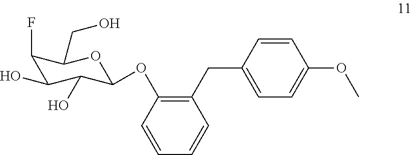

200 mg (0.94 mmol) of 2-(4-methoxybenzyl)phenol 7 and 200 mg (0.63 mmol) of bromide 4 are reacted in analogy to the synthesis of compound 9, and 110 mg of 11 are obtained as a colorless solid. C₂₀H₂₃FO₆ (378.40) MS (ESI⁻) 423.22 (M+CHO₂⁻).

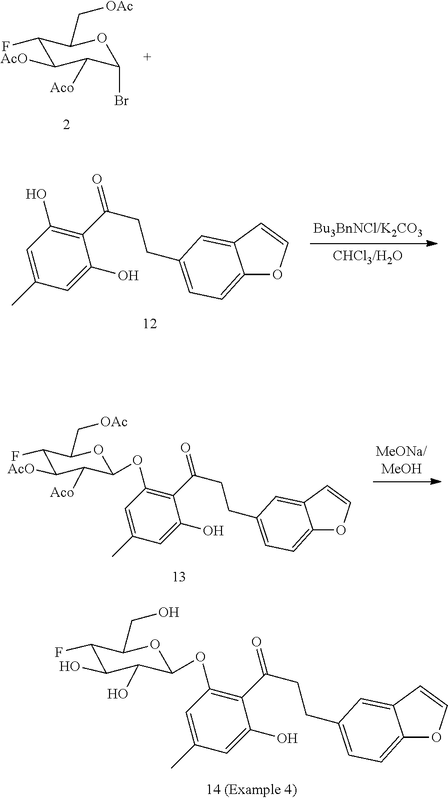

The following were prepared in an analogous manner:

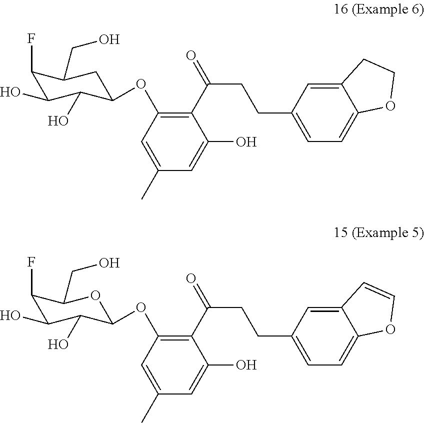

### Example 4

### Compound 14

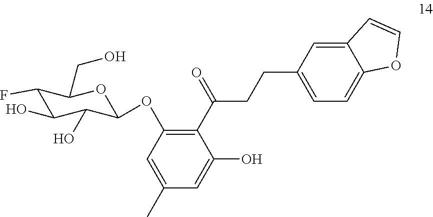

90 mg (0.30 mmol) of 3-benzofuran-5-yl-1-(2,6-dihydroxy-4-methylphenyl)propan-1-one 12 and 280 mg (0.76 mmol) of bromide 2 are reacted in analogy to the synthesis of compound 8, and 400 mg of 13 are obtained as crude product which is directly deprotected with NaOMe/MeOH in analogy to the synthesis of glucoside 9. 75 mg of 14 (54% over 2 stages) are obtained as a colorless solid. C₂₄H₂₅FO₈ (460.46) MS (ESI⁻) 459.03 (M−H⁺).

### Example 5

### Compound 15

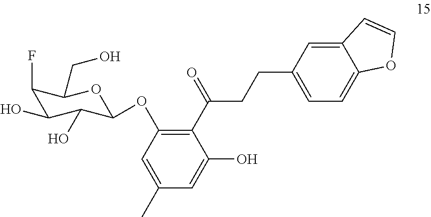

100 mg (0.33 mmol) of 3-benzofuran-5-yl-1-(2,6-dihydroxy-4-methylphenyl)propan-1-one 12 and 150 mg (0.40 mmol) of bromide 4 are reacted in analogy to the synthesis of compound 14, and 75 mg of 15 are obtained as a colorless solid. C₂₄H₂₅FO₈ (460.46) MS (ESI⁻) 459.03 (M−H⁺).

### Example 6

### Compound 16

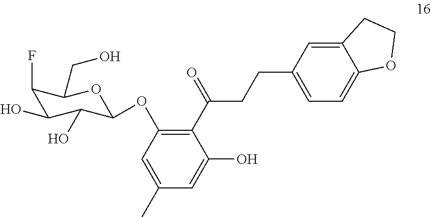

150 mg (0.5 mmol) of 3-(2,3-dihydroxybenzofuran-5-yl-1-(2,6-dihydroxy-4-methylphenyl)propan-1-one and 150 mg (0.40 mmol) of bromide 4 are reacted in analogy to the synthesis of compound 14, and 75 mg of 16 are obtained as a colorless solid. C₂₄H₂₇FO₈ (462.46) MS (ESI⁻) 461.03 (M−H⁺).

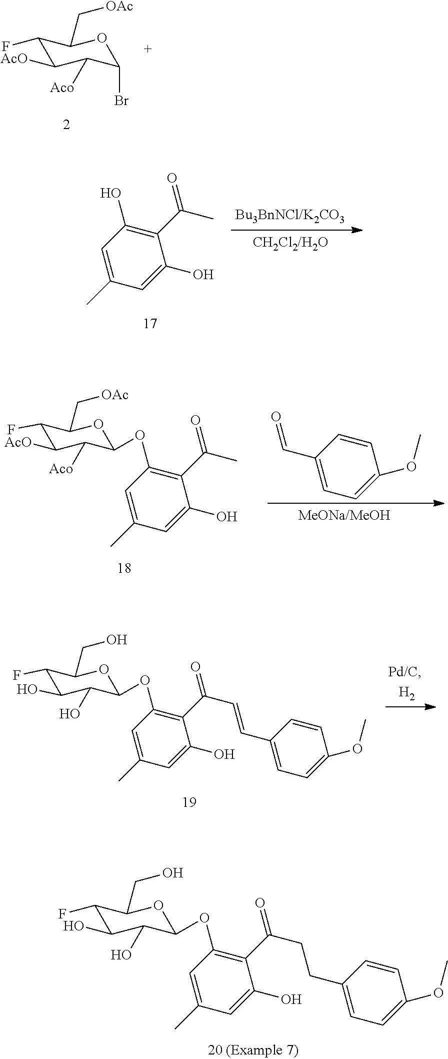

The following was prepared in an analogous manner:

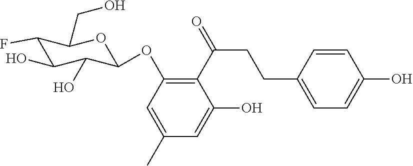

### Example 7

### Compound 20

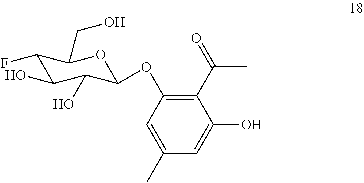

1.0 g (6.0 mmol) of 1-(2,6-dihydroxy-4-methylphenyl)ethanone 17 and 1.0 g (2.7 mmol) of bromide 2 are dissolved in 30 ml of methylene chloride. 800 mg of benzyltributylammonium chloride (PTC), 1.6 g of potassium carbonate and 1.5 ml of water are successively added to this solution while stirring vigorously. This suspension is stirred with protection from light (aluminum foil) for 18 hours and then diluted with 150 ml of ethyl acetate and 150 ml of n-heptane. The solid constituents are filtered through a little silica gel and concentrated. The residue is separated by chromatography on silica gel (ethyl acetate/heptane=1/2). 430 mg of 18 are obtained as a pale yellow solid (can be separated with difficulty from an identically migrating byproduct, and thus the purity is only about 50%. The byproduct can easily be removed at the next stage). O₂₁H₂₅O₁₀F (456.43) MS (ESI⁻): 455.25 (M−H⁺).

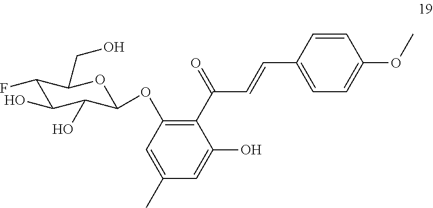

200 mg of compound 18 (about 50% pure) and 225 mg of anisaldehyde (Fluka) are dissolved in 10 ml of methanol. After addition of 5 ml of 1N NaOMe/MeOH solution, the reaction solution is boiled under reflux for 12 hours. The reaction solution is neutralized with methanolic HCl and concentrated, and the residue is separated by chromatography on silica gel (methylene chloride/methanol/conc. ammonia, 30/5/1). 60 mg of 19 are obtained as a yellow solid.

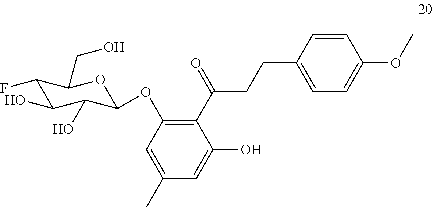

60 mg (0.13 mmol) of chalcone 19 and 50 mg of Pd/C (10% Pd) are suspended in 15 ml of methanol and hydrogenated under a 5 bar hydrogen atmosphere at room temperature for 5 h. The reaction solution is concentrated and the residue is purified by flash chromatography (methylene chloride/methanol/conc. ammonia, 30/5/1).

Yield 25 mg (42%) of 20 as a white amorphous solid. C₂₃H₂₇FO₈ (424.47) MS (ESI⁻): 449.17 (M−H⁺).

### Example 8

### Compound 21

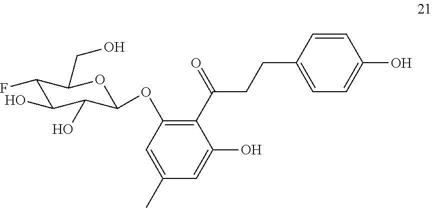

200 mg of compound 18 (about 50% pure) and 350 mg of p-benzyloxybenzaldehyde (Fluke) are reacted in analogy to the synthesis of compound 20. 36 mg of 21 are obtained as a colorless solid. C₂₂H₂₅FO₈ (436.44) MS (ESI⁻) 481.08 (M+CHO₂⁻).

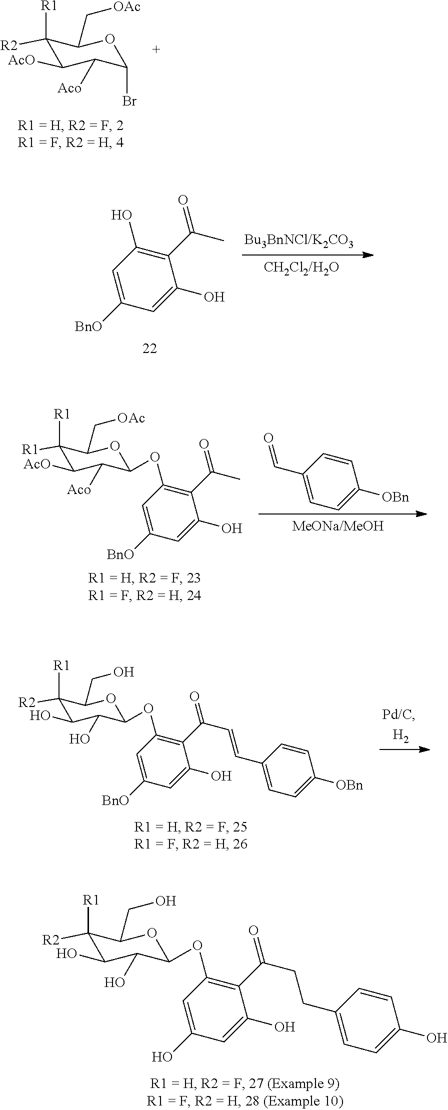

### Example 9

### Compound 27

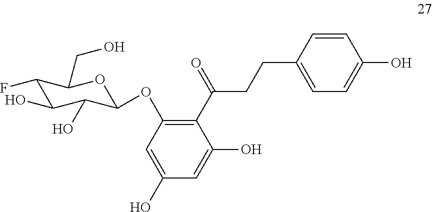

350 mg of bromide 2, 100 mg of phenol 22 and 350 mg of p-benzyloxybenzaldehyde (Fluka) are reacted in analogy to the synthesis of compound 21. 40 mg of 27 are obtained as a colorless solid. C₂₁H₂₃FO₉ (438.41) MS (ESI⁻) 483.15 (M+CHO₂⁻).

### Example 10

### Compound 28

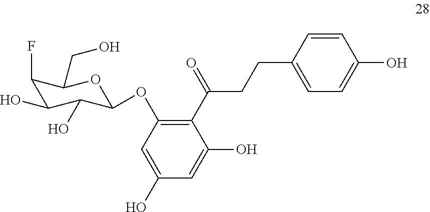

110 mg of bromide 4 80 mg of phenol 22 and 350 mg of p-benzyloxybenzaldehyde (Fluka) are reacted in analogy to the synthesis of compound 21. 50 mg of 28 are obtained as a colorless solid. C₂₁H₂₃FO₉ (438.41) MS (ESI⁻) 483.15 (M+CHO₂⁻).

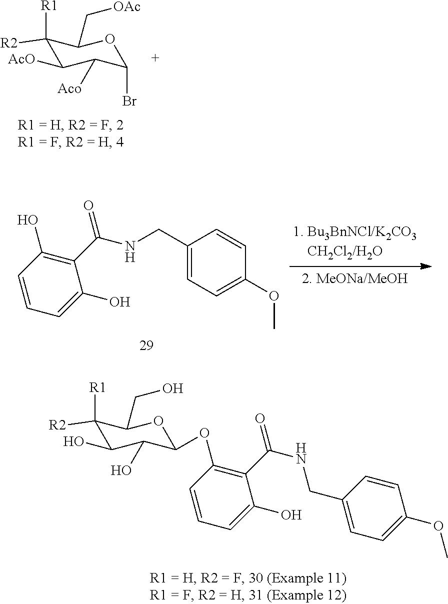

### Example 11

### Compound 30

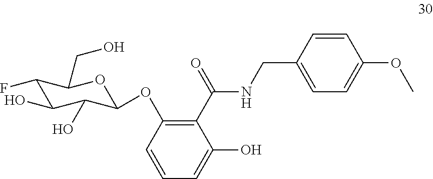

200 mg of bromide 2 and 300 mg of phenol 29 are reacted in analogy to the synthesis of compound 14. 40 mg of 30 are obtained as a colorless solid. O₂₁H₂₄FNO₈ (437.43) MS (ESI⁻) 482.15 (M+CHO₂⁻).

### Example 12

### Compound 31

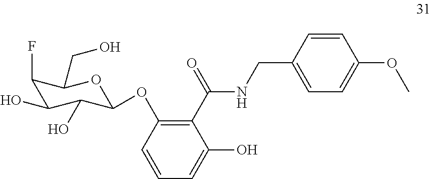

200 mg of bromide 4 and 300 mg of phenol 29 are reacted in analogy to the synthesis of compound 14. 115 mg of 31 are obtained as a colorless solid. C₂₁H₂₄FNO₈ (437.43) MS (ESI⁻) 482.15 (M+CHO₂⁻).

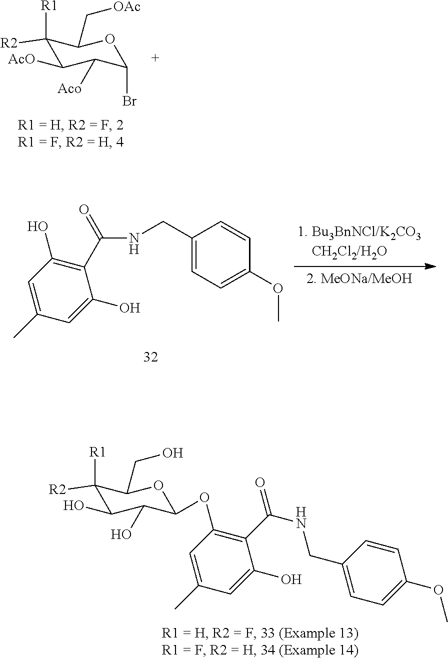

### Example 13

### Compound 33

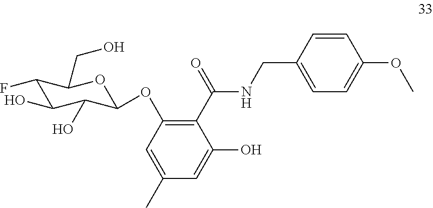

200 mg of bromide 2 and 300 mg of phenol 32 are reacted in analogy to the synthesis of compound 14. 80 mg of 33 are obtained as a colorless solid. C₂₂H₂₆FNO₈ (451.45) MS (ESI⁻) 496.17 (M+CHO₂⁻).

### Example 14

### Compound 34

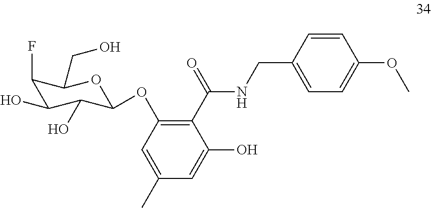

200 mg of bromide 4 and 300 mg of phenol 32 are reacted in analogy to the synthesis of compound 14. 130 mg of 34 are obtained as a colorless solid. C₂₁H₂₄FNO₈ (451.45) MS (ESI⁻) 496.15 (M+CHO₂⁻).

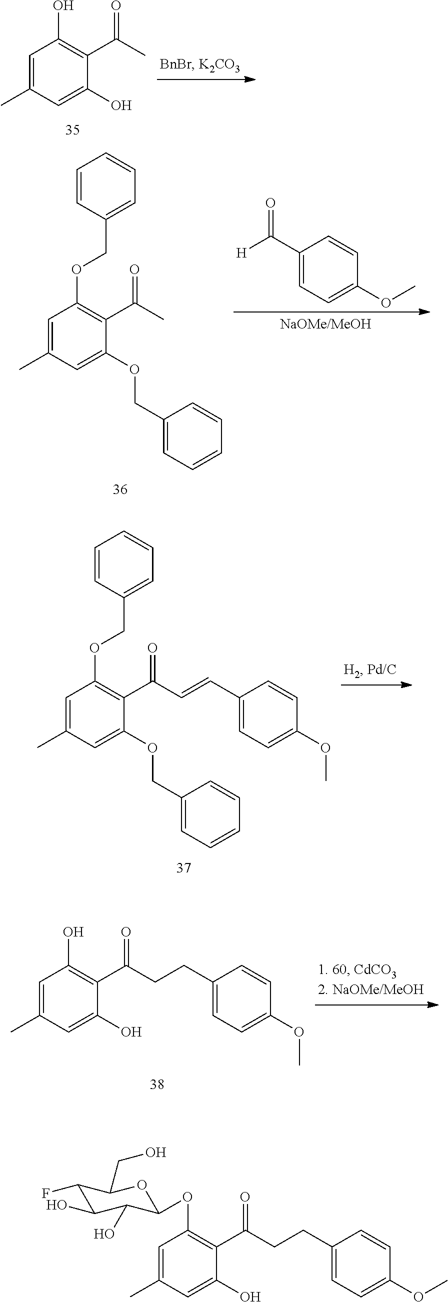

### 1-(2,6-Bisbenzyloxy-4-methylphenyl)ethanone 36

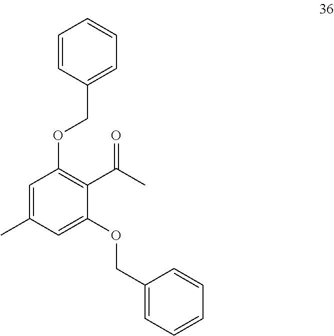

1.62 g (9.75 mmol) of 1-(2,6-dihydroxy-4-methylphenyl)ethanone (35) are dissolved in 30 ml of dimethylformamide, and 4.0 ml (33.7 mmol) of benzyl bromide and 13.8 g (100 mmol) of potassium carbonate are added. The reaction mixture is stirred at room temperature for 3 hours. This is followed by addition of water and extraction twice with ethyl acetate. The combined organic phases are washed with saturated sodium chloride solution, dried over sodium sulfate and concentrated in a rotary evaporator. 1.35 g (40%) of compound 36 are obtained as a colorless crystalline product. C₂₃H₂₂O₃ (346.2) MS (ESI⁺): 347.15 (M+H⁺).

### 1-(2,6-Bisbenzyloxy-4-methylphenyl)-3-(4-methoxyphenyl)propenone 37

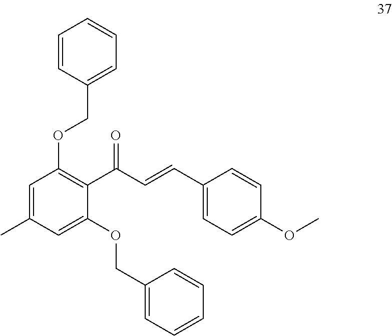

3.46 g (10 mmol) of 1-(2,6-bisbenzyloxy-4-methylphenyl)ethanone (36) are dissolved in 150 ml of ethanol, and 1.34 ml of p-anisaldehyde are added. 7 ml of aqueous potassium hydroxide solution are then added dropwise. The reaction stirs at room temperature for 12 hours.

Half of the solvent is stripped off in a rotary evaporator. The mixture is neutralized with 2 M hydrochloric acid while cooling in ice and is then extracted three times with water and ethyl acetate. The organic phases are combined, washed with saturated sodium chloride solution, dried over sodium sulfate and concentrated. The isolated oil crystallizes out. The crystals are stirred in diethyl ether, filtered off with suction and dried. 4.3 g (92%) of the compound 37 are obtained as a colorless solid. g/mol C₃₁H₂₈O₄ (464.2) MS (ESI⁺): 465.10 (M+H⁺).

### 1-(2,6-Dihydroxy-4-methylphenyl)-3-(4-methoxyphenyl)propan-1-one 38

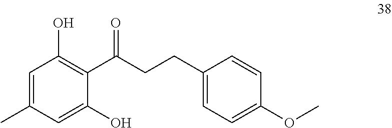

1.50 g (3.23 mmol) of 1-(2,6-bisbenzyloxy-4-methylphenyl)ethanone (37) are dissolved in 40 ml of ethyl acetate and, under an argon atmosphere, 400 mg of palladium on activated carbon, 10%, are added. Hydrogenation is carried out in a hydrogenation autoclave under 3 bar at room temperature for 2 hours. The catalyst is then filtered off and washed with ethyl acetate, and the resulting solution is concentrated in a rotary evaporator. The crude product is purified by column chromatography (SiO₂, ethyl acetate/n-heptane 1:3).

600 mg of the product 38 (65%) are isolated as a colorless solid. C₁₇H₁₈O₄ 286.3 MS (ESI⁺): 287.10 (M+H⁺).

### Reference Example 7

### Compound 20

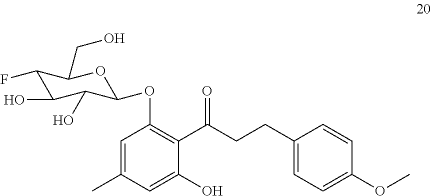

174.4 mg (0.61 mmol) of compound 38 are dissolved in 50 ml of toluene, and 340 mg (0.61 mmol) of the bromide 60 and 421 mg of cadmium carbonate (2.44 mmol) are added. The reaction mixture is refluxed with a water trap for 1 h. Cadmium carbonate is filtered off, and the resulting clear solution is concentrated in a rotary evaporator. The crude product is suspended in 25 ml of methanol and mixed with 5.0 ml of a 0.5 M methanolic NaOMe solution and stirred at room temperature for 12 h. The reaction solution is neutralized by adding methanolic HCl and is purified by flash chromatography (SiO2, EtOAc/heptane 1:4→1:1). 78.8 mg (29%) of compound 20 are obtained as a colorless solid. C₂₃H₂₇FO₈ 450.5 MS (ESI⁺): 473.15 (M+Na⁺).

Compounds 40 (example 15), 41 (example 16), 42 (example 17) and 43 (example 18) were prepared in an analogous manner.

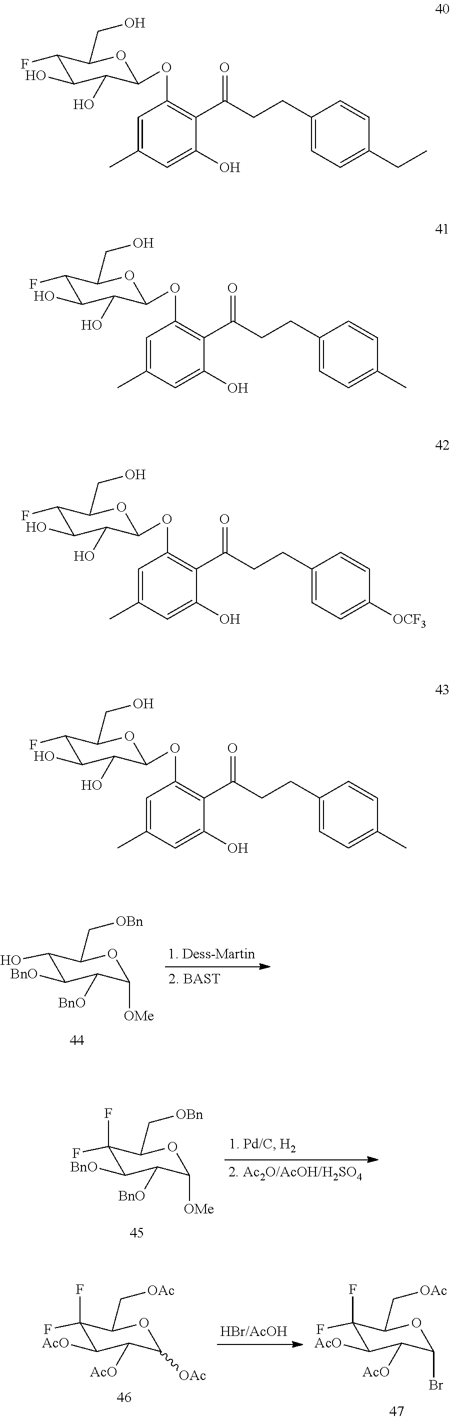

### 1-Methoxy-4-deoxy-4,4-difluoro-2,3,6-tri-O-benzyl-alpha-D-glucose 45

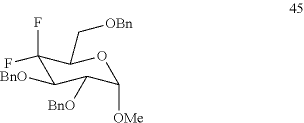

3.69 g (7.9 mmol) of 1-methoxy-2,3,6-tri-O-benzyl-α-D-glucose 44 (Tetrahedron Asymmetry 11 (2000) 385-387) are dissolved in 110 ml of methylene chloride and, under an argon atmosphere, 3.6 g (8.5 mmol) of Dess-Martin agent (Aldrich) are added dropwise. After 3 hours at room temperature, the mixture is diluted with 300 ml of ethyl acetate/n-heptane (1:1) and washed 1× with NaHCO₃ solution and 1× with Na₂S₂O₃ solution. The organic phase is filtered through silica gel and concentrated. The residue is separated by chromatography on silica gel (ethyl acetate/n-heptane 1:1). 2.9 g (79%) of ketone are obtained. The latter is dissolved in 30 ml of methylene chloride and, under an argon atmosphere, 4 ml of BAST (Aldrich) are added dropwise. After 20 hours at room temperature, the mixture is diluted with 200 ml of ethyl acetate and washed cautiously (strong effervescence) with cooled NaHCO₃ solution. The organic phase is filtered through silica gel and concentrated. The residue is separated by chromatography on silica gel (ethyl acetate/n-heptane 1:1). 2.6 g (85%) of 45 are obtained as a colorless oil.

### 4-Deoxy-4,4-difluoro-1,2,3,6-tetra-O-acetyl-alpha-D-glucose 46

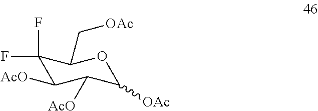

2.3 g (4.7 mmol) of 45 and 2 g of Pd/C (10% Pd) are dissolved in 150 ml of methanol and 10 ml of acetic acid and hydrogenated under an atmosphere of 5 bar of hydrogen at room temperature for 16 h. The reaction solution is concentrated and the residue is purified by flash chromatography (methylene chloride/methanol/conc. ammonia, 30/5/1). Yield 850 mg (83%) of 1-methoxy-4-deoxy-4,4-difluoro-alpha-D-glucose as white amorphous solid. C₇H₁₂F₂O₅ (214.17) MS (DCI): 215.4 (M+H⁺).

700 mg (3.3 mmol) of this are dissolved in 3.5 ml of acetic acid and 6.3 ml of acetic anhydride. Addition of 0.2 ml of conc. H₂SO₄ is followed by stirring at 60° C. for 5 h. The reaction solution is then poured into a mixture of 30 g of ice and 30 ml of ethyl acetate.

The organic phase is washed twice more with aqueous NaCl solution, filtered through a little silica gel and concentrated. The residue is separated by chromatography on silica gel (ethyl acetate/n-heptane 1:1). 300 mg (25%) of 46 are obtained as a mixture of anomers. C₁₄H₁₈F₂O₉ (368.29) MS (DCI): 369.3 (M+H⁺).

### 1-Bromo-4-deoxy-4,4-difluoro-2,3,6-tri-O-acetyl-alpha-D-glucose 47

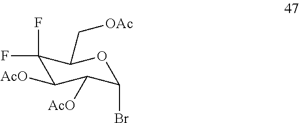

300 mg (0.8 mmol) of tetraacetate 46 are dissolved in 13 ml of 33% strength HBr in glacial acetic acid and left to stand at room temperature for 6 hours. The reaction solution is then poured into a mixture of 10 g of ice and 10 ml of ethyl acetate. The organic phase is washed twice more with aqueous NaCl solution, filtered through a little silica gel and concentrated. The residue is separated by chromatography on silica gel (ethyl acetate/heptane 1:1). 112 mg (35%) of 47 are obtained as a colorless solid. C₁₂H₁₅BrF₂O₇ (389.15) MS (DCI): 389.2 (M+H⁺).

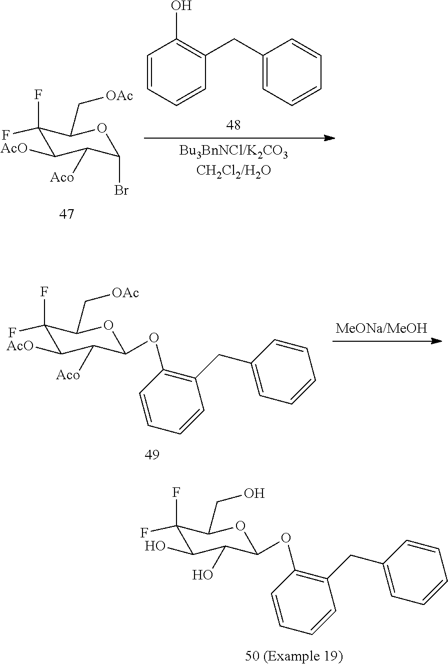

### Example 19

### Compound 50

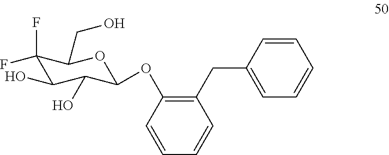

100 mg (0.47 mmol) of 2-benzylphenol (Aldrich) and 40 mg (0.10 mmol) of difluoro bromide 47 are reacted in analogy to the synthesis of compound 9, and 21 mg of 50 are obtained as a colorless solid. C₁₉H₂₀F₂O₅ (366.37) MS (ESI⁻) 411.15 (M+CHO₂⁻).

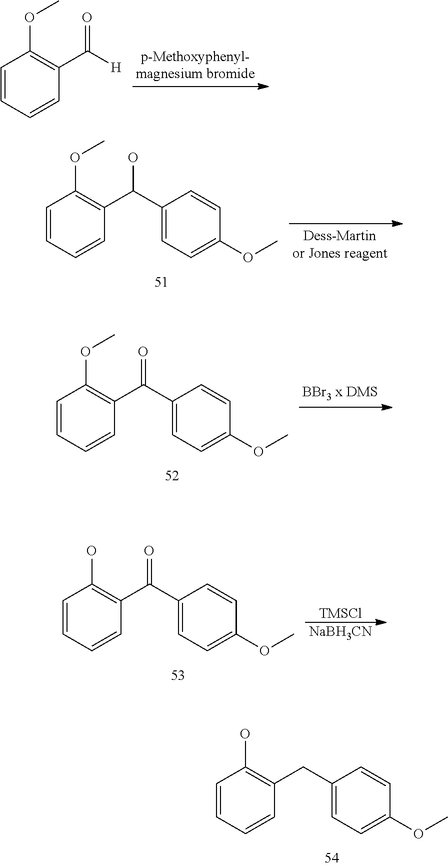

### (4-Methoxyphenyl)-(2-methoxyphenyl)methanol 51

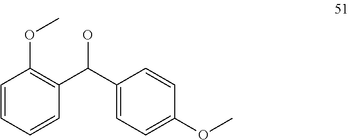

1.5 g of o-anisaldehyde are dissolved in THF and cooled to 0° C. 24.2 ml of 4-methoxyphenylmagnesium bromide (0.5 M in THF) are added to the mixture. The reaction solution is stirred at room temperature overnight and then poured into a 20% NH₄Cl solution and extracted with ethyl acetate. 2.63 g of the product are obtained, and this can be employed without further purification. C₁₅H₁₆O₃ (244.29) MS (ESI⁺) 227.05 (M-OH)⁺

### (4-Methoxyphenyl)-(2-methoxyphenyl)methanone 52

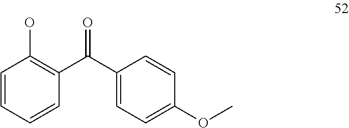

2.63 g of (4-methoxyphenyl)(2-methoxyphenyl)methanol 51 are dissolved in dichloromethane, and 5.03 g of Dess-Martin reagent are added. The mixture is stirred at room temperature for 2 h. Then 20% Na₂SO₃ and NaHCO₃ solution are added, and the mixture is extracted with diethyl ether. The organic phase is extracted with saturated NaCl solution and dried over sodium sulfate. The solution is concentrated in vacuo and purified by column filtration. 2.61 g of 52 are obtained. C₁₅H₁₄O₃ (242.28) MS (ESI⁺) 243.04 (M+H⁺)

Oxidation with Jones Reagent can Take Place as an Alternative Thereto:

155 mg of (4-methoxyphenyl)(2-methoxyphenyl)methanol 51 are dissolved in 10 ml of acetone, and 2 ml of Jones reagent are added dropwise. After 2 h at room temperature, 50 l of MTB ether and 30 ml of water are added to the mixture. The organic phase is washed several times with water, and the organic phase is extracted with saturated NaCl solution, dried over sodium sulfate and evaporated to dryness. The product (126 mg) obtained in this way has sufficient purity for further reaction.

### (2-Hydroxyphenyl)(4-methoxyphenyl)methanone 53

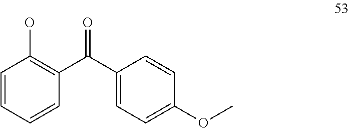

2.61 g of (4-methoxyphenyl)(2-methoxyphenyl)methanone 52 are dissolved in dichloromethane. The mixture is cooled in an ice bath, and 3.71 g of boron tribromide-dimethyl sulfide complex are added. The mixture is warmed to room temperature and left to stir for 3 h. The reaction is then stopped by pouring into ice-water, the dichloromethane phase is separated off, and the aqueous phase is extracted several times with ethyl acetate. The combined organic phase is washed with water and sodium chloride solution, dried over sodium sulfate and concentrated. The crude product is chromatographed on silica gel with ethyl acetate/heptane. 1.26 g of the product are obtained. C₁₄H₁₂O₃ (228.25) MS (DCI) 229.2 (M+H⁺)

### 2-(4-Methoxybenzyl)phenol 7

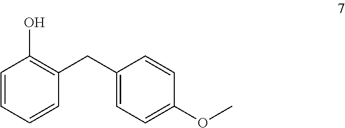

0.78 g of (2-hydroxyphenyl)(4-methoxyphenyl)methanone is dissolved in acetonitrile and cooled to 0° C. 2 ml of TMSCl are added dropwise to the mixture, and then 1 g of sodium cyanoborohydride is added. The mixture is stirred at room temperature for 3 h. The reaction solution is diluted with dichloromethane and filtered through Celite. The organic phase is washed with water and saturated sodium chloride solution, dried over sodium sulfate and concentrated in vacuo. The crude product is chromatographed on silica gel with ethyl acetate/heptane (1/2). 0.72 g of the desired product is obtained. C₁₄H₁₄O₂ (214.27) MS (ESI⁺): 232.20 (M+NH₄⁺)⁺

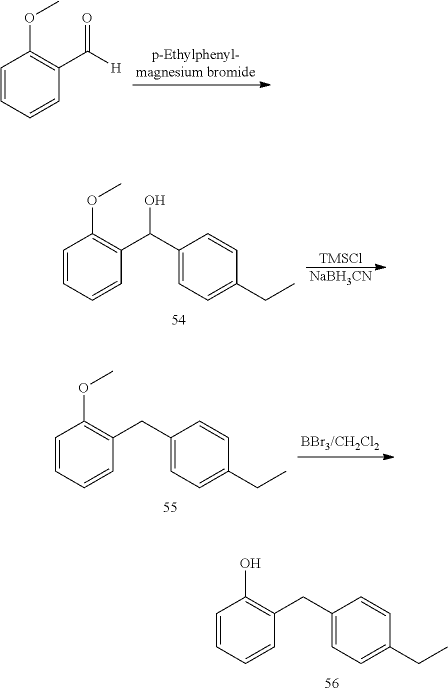

### (4-Ethylphenyl)(2-methoxyphenyl)methanol 54

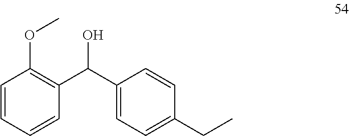

1.01 g of o-anisaldehyde are dissolved in THF and cooled to 0° C. 16.29 ml of 4-ethylphenylmagnesium bromide (0.5 M in THF) are added to the mixture. The reaction solution is stirred at room temperature overnight and then poured into 20% NH₄Cl solution and extracted with ethyl acetate. 1.92 g of the product are obtained, and this can be employed without further purification. C₁₆H₁₈O₂ (242.32) MS (ESI⁺) 225.15 (M-OH)⁺

### 4-Ethylphenyl)(2-methoxyphenyl)methane 55

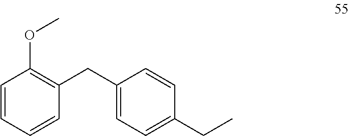

1.34 g of (4-ethylphenyl)(2-methoxyphenyl)methanol are dissolved in acetonitrile and cooled to 0° C. 1.50 g of sodium cyanoborohydride are added to the mixture and then 3.00 ml of trimethylsilyl chloride are added. The mixture is stirred at room temperature overnight. The reaction solution is filtered through Celite and extracted with saturated NaCl solution. The organic phase is dried over sodium sulfate and concentrated. The crude product is chromatographed on silica gel with ethyl acetate/heptane (1/12). 0.83 g of the product is obtained. C₁₆H₁₈O (226.32) MS (DCI) 227.4 (M+H⁺)

### 2-(4-Ethylbenzyl)phenol 56

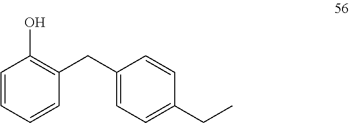

0.83 g of (4-ethylphenyl)(2-methoxyphenyl)methane 55 is dissolved in dichloromethane. 11.0 ml of boron tribromide (1 M in CH₂Cl₂) are added dropwise to the mixture. The mixture is stirred at room temperature for 5 hours and, after addition of water, the dichloromethane phase is separated off. The aqueous phase is extracted with ethyl acetate. The combined organic phases are washed with water and NaCl solution, dried over sodium sulfate and concentrated. 0.77 g is obtained as crude product which can be purified by chromatography. C₁₅H₁₆O (212.29) MS (ESI): 235.20 (M+Na⁺)

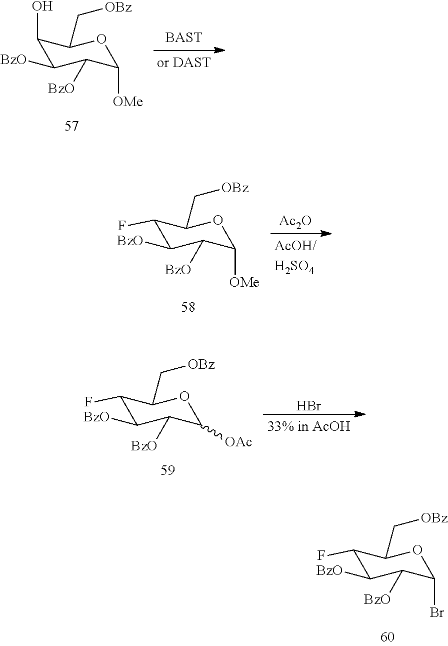

### Methyl 2,3,6-tri-O-benzoyl-4-fluoro-4-deoxy- -D-glucopyranoside 58

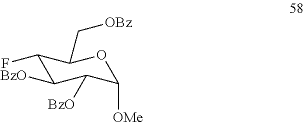

3 g of methyl 2,3,6-tri-O-benzoyl- -D-galactopyranoside 57 (Reist et al., J. Org. Chem. 1965, 30, 2312) are introduced into dichloromethane and cooled to −30° C. Then 3.06 ml of [bis(2-methoxyethyl)amino]sulfur trifluoride (BAST) are added dropwise. The reaction solution is warmed to room temperature and stirred overnight. The mixture is diluted with dichloromethane, and the organic phase is extracted with H₂O, NaHCO₃ solution and saturated NaCl solution. The organic phase is dried over Na₂SO₄ and concentrated. The crude product is crystallized from ethyl acetate and heptane. 1.95 g of 58 are obtained as a colorless solid. C₂₈H₂₅FO₈ (508.51) MS (ESI⁺) 526.18 (M+NH₄⁺). Alternatively, the reaction can also be carried out using 2.8 eq. of diethylaminosulfur trifluoride (DAST); in this case, the reaction solution is refluxed for 18 h after the addition. The working up takes place in analogy to the above description.

### 1-O-Acetyl-2,3,6-tri-O-benzoyl-4-fluoro-4-deoxyglucose 59

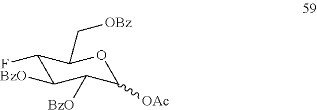

12 g of methyl 2,3,6-tri-O-benzoyl-4-fluoro-4-deoxy- -D-glucopyranoside 58 are suspended in 150 ml of acetic anhydride. 8.4 ml of conc. sulfuric acid are mixed with 150 ml of glacial acetic acid and added to the mixture while cooling in ice. The mixture stirs at room temperature for 60 h. The mixture is poured into NaHCO₃ solution, and this solution is extracted with dichloromethane. The organic phase is extracted with NaCl solution, dried with Na₂SO₄ and concentrated. The residue is recrystallized from ethyl acetate/heptane. 5.97 g of the product are obtained as a colorless solid. C₂₉H₂₅FO₉ (536.52) MS (ESI⁺) 554.15 (M+NH₄⁺)

### 2,3,6-Tri-O-benzoyl-4-fluoro-4-deoxyglucosyl bromide 60

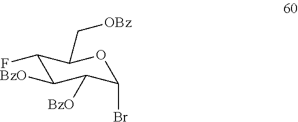

1.44 g of 1-O-acetyl-2,3,6-tri-O-benzoyl-4-fluoro-4-deoxyglucose are dissolved in 20 ml of hydrobromic acid in glacial acetic acid (33%) and stirred at room temperature. After 5 hours, the mixture is poured into ice-water, and the aqueous phase is extracted three times with dichloromethane. The collected organic phase is extracted with saturated sodium chloride solution, dried over sodium sulfate and evaporated to dryness. The crude product is filtered through a silica gel column with ethyl acetate/heptane 70:30. 1.40 g of the product are obtained as a solid. C₂₇H₂₂BrFO₇ (557.37) MS (ESI⁺) 574.05/576.05 (M+NH₄⁺)

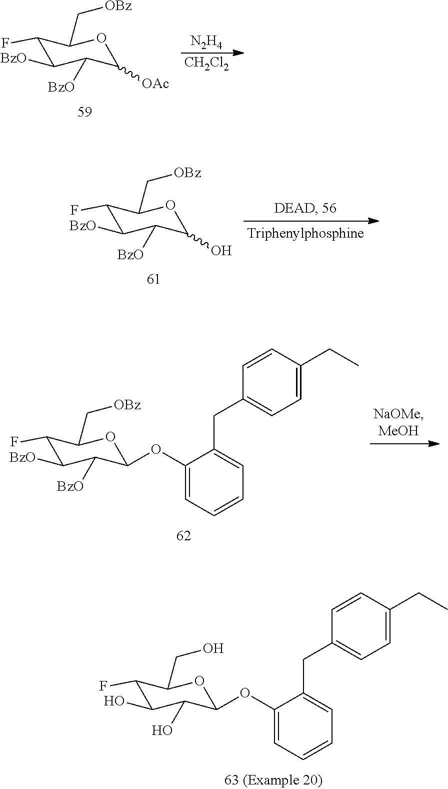

### 2,3,6-Tri-O-benzoyl-4-fluoro-4-deoxyglucose 61

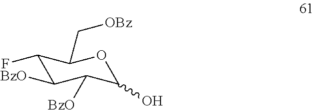

1.60 g of 1-O-acetyl-2,3,6-tri-O-benzoyl-4-fluoro-4-deoxyglucose are dissolved in dichloromethane. 173 μl of hydrazine hydrate are added to this solution. After 16 h, the reaction solution is partitioned between dichloromethane and H₂O. The organic phase is extracted with NaCl solution, dried over sodium sulfate and evaporated to dryness. The crude product is purified by column filtration. 1.22 g of the desired product are obtained. C₂₇H₂₃FO₈ (494.48) MS (ESI⁺): 512.15 (M+NH₄⁺).

### Compound 62

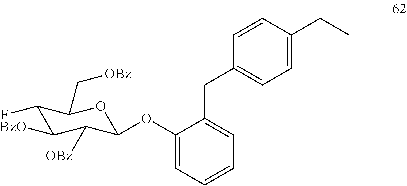

248 mg of 2-(4-ethylbenzyl)phenol (56), 550 mg of 2,3,6-tri-O-benzoyl-4-fluoro-4-deoxyglucose (61) and 335 mg of triphenylphosphine in 2 ml of dry dichloromethane are cooled to 0° C. under argon. 0.193 ml of diethyl azodicarboxylate is slowly added dropwise. This solution is brought to room temperature and stirs overnight. The solution is then diluted with dichloromethane and extracted with water, 0.5 M NaOH and saturated NaCl solution. The organic phase is dried over sodium sulfate and concentrated in vacuo. The residue is purified by chromatography (heptane:ethyl acetate 3:1). 200 mg of the desired product are obtained. C₄₂H₃₇FO₈ (688.76) MS (ESI): 706.30 (M+NH₄)⁺.

### Example 20

### Compound 63

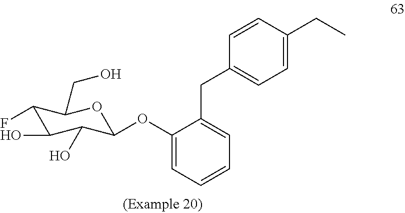

200 mg of 62 are taken up in 10 ml of absolute methanol, and 1 ml of sodium methanolate solution (10 mg of sodium methanolate per ml of methanol) is added. The solution stirs for 8 h. Sodium is removed by adding Amberlyst 15 (H⁺ form), the ion exchanger is filtered off, and the residue is thoroughly washed. The resulting product is purified by silica gel filtration (dichloromethane:methanol 96:4). 56 mg of the desired product are obtained. O₂₁H₂₅FO₅ (376.43) MS (ESI): 394.25 (M+NH₄⁺)

The following examples are prepared in an analogous manner to example 20 using the appropriate aglycones:

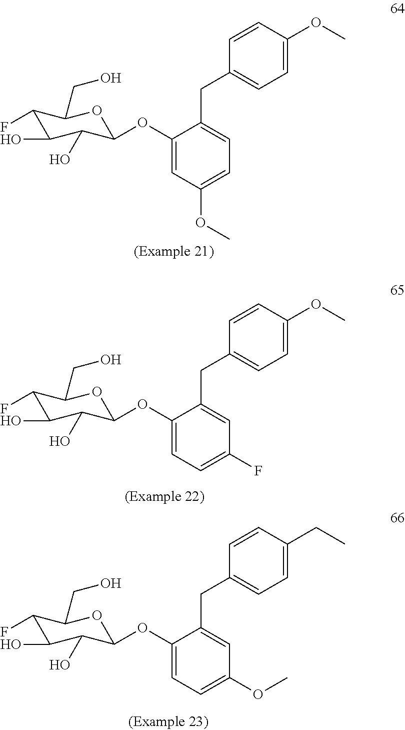

The appropriate aglycones can be obtained for example by the processes described for compounds 7 or 56.

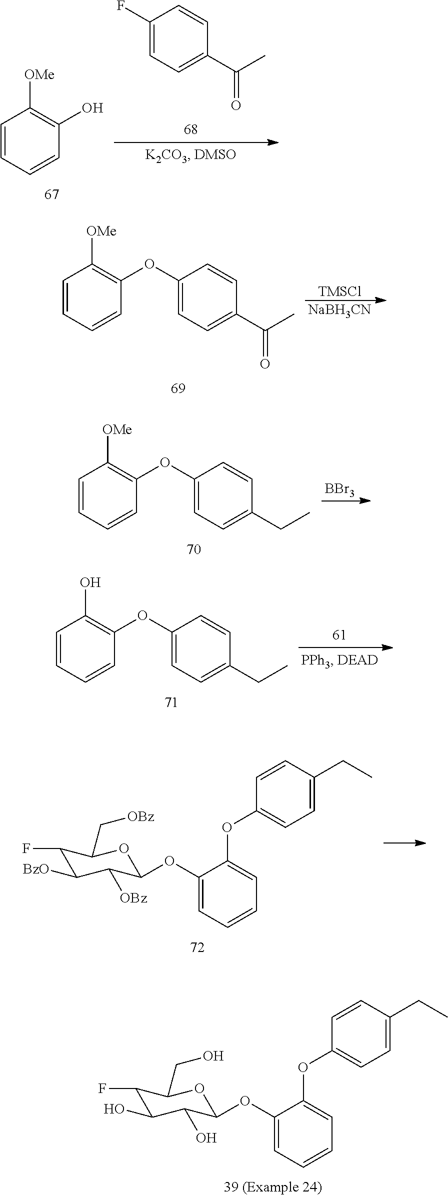

### 1-[4-(2-Methoxyphenoxy)phenyl]ethanone 69

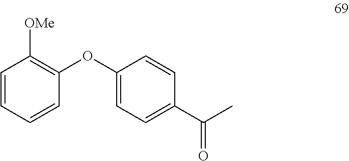

0.15 ml of guaiacol 67, 167 mg of 4-fluoroacetophenone 68 and 335 mg of potassium carbonate are heated in 5 ml of dimethyl sulfoxide at 170° C. in a microwave for 10 min. The reaction solution is poured into water, and the emulsion is extracted three times with an ethyl tert-butyl ether. The combined organic phase is extracted twice with 1 N NaOH and once with saturated NaCl solution, dried and concentrated in vacuo.

240 mg of the desired product are obtained. C₁₅H₁₄O₃ (242.28) MS (ESI): 215.10 (M+H⁺).

### 2-(4-Ethylphenoxy)methoxybenzene 70

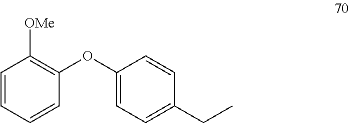

960 mg of 1-[4-(2-methoxyphenoxy)phenyl]ethanone 69 are dissolved in 20 ml of acetonitrile and cooled in an ice bath, and 1.05 g of sodium cyanoborohydride and 2.01 ml of trimethylsilyl chloride are added. After 1 h, the mixture is diluted with dichloromethane and filtered through Celite, and the organic phase is extracted with sodium chloride solution, dried over sodium sulfate and concentrated. The residue is purified by chromatography (heptane:ethyl acetate 7:1). 710 mg of the desired product are obtained. C₁₅H₁₆O₂ (228.29) MS (ESI): 246.20 (M+NH₄⁺).

### 2-(4-Ethylphenoxy)phenol 71

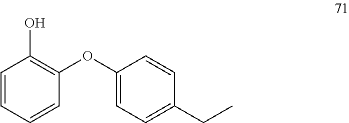

710 mg of 2-(4-ethylphenoxy)methoxybenzene 70 are dissolved in 5 ml absolute dichloromethane. 0.6 ml of boron tribromide (1 M dichloromethane) is added dropwise, and the solution stirs for 6 h. Further BBr₃ is added and the mixture is stirred until the reaction is almost complete according to LCMS. The solution is brought into ice-water, the organic phase is separated off, and the aqueous phase is extracted three times with dichloromethane. The combined organic phase is dried, evaporated to dryness and purified by chromatography. 450 mg of the desired product are obtained. C₁₄H₁₄O₂ (214.27) MS (ESI): 215.10 (M+H⁺).

### Compound 72

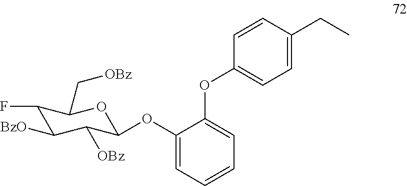

Compound 61 (466 mg) and phenol 71 (242 mg) are reacted in analogy to the synthesis of compound 62. The resulting product can be purified by column chromatography (heptane:ethyl acetate 4:1). 240 mg of the desired product are obtained. C₄₁H₃₅FO₉ (690.73) MS (ESI): 708.25 (M+NH₄⁺).

### Example 24

### Compound 39

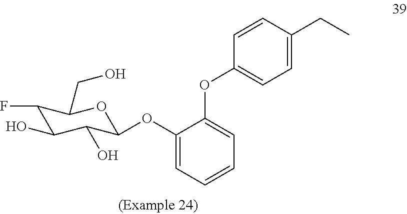

230 mg of compound 72 are reacted with sodium methanolate in analogy to the liberation of example 20. The compound can be purified by silica gel chromatography (dichloromethane:methanol 96:4). 119 mg of the desired product are obtained. C₂₀H₂₃FO₆ (378.40) MS (ESI): 396.15 (M+NH₄⁺).

1. A pharmaceutical composition comprising a compound with SGLT-1/SGLT-2 inhibitor activity for the prevention and/or treatment of osteoporosis.

2. A pharmaceutical composition comprising a compound of formula I 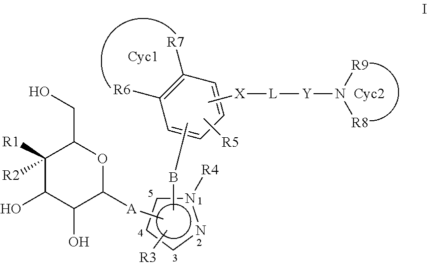
in which the meanings are
R1 and R2 independently of one another F or H, where one of the radicals R1 or
R2 must be F;
A O, NH, CH₂, S or a bond;
R3 hydrogen, F, Cl, Br, I, OH, CF₃, NO₂, CN, COOH, CO—(C₁-C₆)-alkyl, COO(C₁-C₆)-alkyl, CONH₂, CONH—(C₁-C₆)-alkyl, CON[(C₁-C₆)-alkyl]₂, (C₁-C₆)-alkyl, (C₃-C₆)-cycloalkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, phenyl, benzyl, (C₁-C₆)-alkoxycarbonyl, where one, more than one or all hydrogen(s) in the alkyl, alkenyl, alkynyl and O-alkyl radicals may be replaced by fluorine;
SO₂—NH₂, SO₂—NH(C₁-C₆)-alkyl, SO₂N[(C₁-C₆)-alkyl]₂, S—(C₁-C₆)-alkyl, 5-(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o may be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH—CO—(C₁-C₇)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
R4 hydrogen, (C₁-C₆)-alkyl, (C₂-C₆)-alkenyl, (C₃-C₆)-cycloalkyl, or phenyl that may optionally be substituted by halogen or (C₁-C₄)-alkyl;
B (C₀-C₁₅)-alkylene, where one or more C atoms of the alkylene radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkylphenyl)- or —NH—;
R5, R6, R7 independently of one another, hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₈)-alkyl, HO—(C₁-C₆)-alkylene, (C₁-C₆)-alkylene-β-(C₁-C₆)-alkyl, where one, more than one, or all hydrogen(s) in the alkyl, alkenyl, alkynyl and O-alkyl radicals may be replaced by fluorine;
SO₂—NH₂, SO₂NH(C₁-C₆)-alkyl, SO_{2N}[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂(C₁-C₆)-alkyl, SO₂—(CH₂)ₒ-phenyl, where o may be 0-6, and the phenyl ring may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH—CO—(C₁-C₆)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₈)-alkyl, (C₁-C₆)-alkyl, NH₂, NH(C₁-C₆)-alkyl, N((C₁-C₆)-alkyl)₂, SO₂—CH₃, COON, COO—(C₁-C₆)-alkyl, CONH₂;
or
R6 and R7 together with the C atoms carrying them a 5 to 7 membered, saturated, partially or completely unsaturated ring Cyc1, where 1 or 2 C atom(s) of the ring may also be replaced by N, O or S, and Cyc1 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
X CO, O, NH, S, SO, SO₂ or a bond;
L (C₁-C₆)-alkylene, (C₂-C₅)-alkenylene, (C₂-C₅)-alkynylene, where in each case one or two CH₂ group(s) may be replaced by O or NH;
Y CO, NHCO, SO, SO₂, or a bond;
R8, R9 independently of one another, hydrogen, SO₃H, sugar residue, (C₁-C₆)-alkyl, where one or more CH₂ groups of the alkyl radical may be substituted independently of one another by (C₁-C₆)-alkyl, OH, (C₁-C₆)-alkylene-OH, (C₂-C₆)-alkenylene-OH, O-sugar residue, OSO₃H, NH₂, NH—(C₁-C₆)-alkyl, N[(C₁-C₆)-alkyl]₂, NH—CO—(C₁-C₆)-alkyl, NH-sugar residue, NH—SO₃H, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂, (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl, adamantyl; or
R8 and R9 together with the N atom carrying them form a 5 to 7 membered, saturated ring Cyc2, where one or more CH₂ groups of the ring may also be replaced by O, S, NH, NSO₃H, N-sugar residue, N—(C₁-C₆)-alkyl, where one or more CH₂ groups of the alkyl radical may be substituted independently of one another by (C₁-C₆)-alkyl, OH, (C₁-C₆)-alkylene-OH, (C₂C₆)-alkenylene-OH, NH₂, NH—(C₁-C₆)-alkyl, N[(C₁-C₆)-alkyl]₂, NH—CO—(C₁-C₆)-alkyl, NH-sugar residue, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂ (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl;
and the pharmaceutically acceptable salts thereof;
or
comprising a compound of formula II 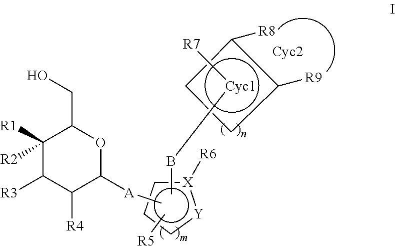
in which the meanings are
R1 and R2 independently of one another F, H or one of the radicals R1 or R2 OH;
R3 OH or F, where at least one of the radicals R1, R2, R3 must be F;
R4 OH;
A O, NH, CH₂, S or a bond;
X C, O, S or N, where X must be C when Y is O or S;
Y N, O or S;
m a number 1 or 2;
R5 hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₆)-alkoxycarboxyl, it being possible for one, more than one or all hydrogen(s) in the alkyl, alkoxy, alkenyl or alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
or when Y is S, R5 and R6 together with the C atoms carrying them phenyl;
R6 optionally H, (C₁-C₆)-alkenyl, (C₃-C₆)-cycloalkyl, or phenyl that may optionally be substituted by halogen or (C₁-C₄)-alkyl;
B (C₀-C₁₅)-alkanediyl, it being possible for one or more C atoms in the alkanediyl radical to be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, it being possible for one, more than one or all hydrogen(s) in the alkyl, alkoxy, alkenyl or alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl; N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO(C₁-C₆)-alkyl, CONH₂;
or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
and the pharmaceutically acceptable salts thereof;
or
comprising a compound of formula III 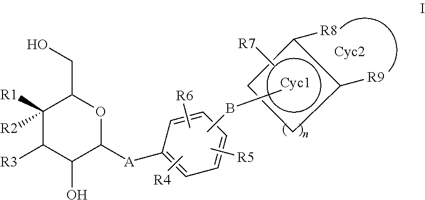
in which the meanings are
R1, R2 OH, F or H or R1 and R2=F, excluding the three combinations R1=F, R2=OH and R1=OH, R2=F and R1, R2=OH;
R3 OH or F, where at least one of the R1, R2, R3 radicals must be F;
A O, NH, CH₂, S or a bond;
R4, R5, R6 hydrogen, F, Cl, Br, I, OH, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO(C₁-C₆)-alkyl, (C₁-C₆)-alkyl-O—(C₁-C₆)-alkyl, phenyl, benzyl, it being possible for one, more than one or all hydrogen(s) in the alkyl, alkoxy, alkenyl and alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
B (C₀-C₁₅)-alkanediyl, it being possible for one or more C atoms in the alkanediyl radical to be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, (C₁-C₆)-alkyl-OH, (C₁-C₆)-alkyl-O—(C₁-C₆)-alkyl, it being possible for one, more than one or all hydrogen(s) in the alkyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO—(C₁-C₄)-alkyl, CONH₂, CONH(C₁-C₄)-alkyl, OCF₃;
and the pharmaceutically acceptable salts thereof
for the prevention and/or treatment of osteoporosis

3. A pharmaceutical composition comprising the following compound 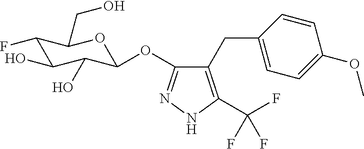
and the pharmaceutically acceptable salts thereof
for the prevention and/or treatment of osteoporosis.

4. A pharmaceutical composition comprising the following compound 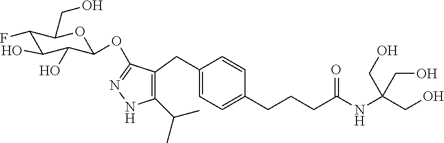
and the pharmaceutically acceptable salts thereof
for the prevention and/or treatment of osteoporosis.

5. (canceled)

6. The pharmaceutical composition of claim 2 comprising the compound of formula I 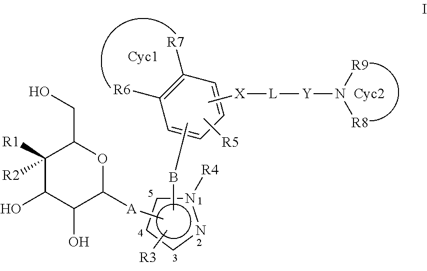
in which the meanings are
R1 and R2 independently of one another F or H, where one of the radicals R1 or R2 must be F;
A O, NH, CH₂, S or a bond;
R3 hydrogen, F, Cl, Br, I, OH, CF₃, NO₂, CN, COOH, CO—(C₁-C₆)-alkyl, COO(C₁-C₆)-alkyl, CONH₂, CONH—(C₁-C₆)-alkyl, CON[(C₁-C₆)-alkyl]₂, (C₁-C₆)-alkyl, (C₃-C₆)-cycloalkyl, (C₂-C₆)-alkenyl, (C₂-C₆)-alkynyl, O—(C₁-C₆)-alkyl, HO—(C₁-C₆)-alkylene, (C₁-C₆)-alkylene-O—(C₁-C₆)-alkyl, phenyl, benzyl, (C₁-C₆)-alkoxycarbonyl, where one, more than one or all hydrogen(s) in the alkyl, alkenyl, alkynyl and O-alkyl radicals may be replaced by fluorine;
SO₂—NH₂, SO₂—NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o may be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH—CO—(C₁-C₇)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
R4 hydrogen, (C₁-C₆)-alkyl, (C₂-C₆)-alkenyl, (C₃-C₆)-cycloalkyl, or phenyl that may optionally be substituted by halogen or (C₁-C₄)-alkyl;
B (C₀-C₁₅)-alkylene, where one or more C atoms of the alkylene radical may be replaced independently of one another by —O—, —(C═O)—, —CH═CH—, —C≡C—, —S—, —CH(OH)—, —CHF—, —CF₂—, —(S═O)—, —(SO₂)—, —N((C₁-C₆)-alkyl)-, —N((C₁-C₆)-alkylphenyl)- or —NH—;
R5, R6, R7 independently of one another, hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, O—(C₁-C₈)-alkyl, HO—(C₁-C₆)-alkylene, (C₁-C₆)-alkylene-β-(C₁-C₆)-alkyl, where one, more than one, or all hydrogen(s) in the alkyl, alkenyl, alkynyl and O-alkyl radicals may be replaced by fluorine;
SO₂—NH₂, SO₂NH(C₁-C₆)-alkyl, SO_{2N}[(C₁-C₆)-alkyl]₂, S—(C₁-C₆)-alkyl, S—(CH₂)ₒ-phenyl, SCF₃, SO—(CH₂)ₒ-phenyl, SO₂(C₁-C₆)-alkyl, SO₂—(CH₂)ₒ-phenyl, where o may be 0-6, and the phenyl ring may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH—CO—(C₁-C₆)-alkyl, phenyl, O—(CH₂)ₒ-phenyl, where o may be 0-6, where the phenyl ring may be substituted one to three times by F, Cl, Br, I, OH, CF₃, NO₂, CN, OCF₃, O—(C₁-C₈)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
or
R6 and R7 together with the C atoms carrying them a 5 to 7 membered, saturated, partially or completely unsaturated ring Cyc1, where 1 or 2 C atom(s) of the ring may also be replaced by N, O or S, and Cyc1 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
X CO, O, NH, S, SO, SO₂ or a bond;
L (C₁-C₆)-alkylene, (C₂-C₅)-alkenylene, (C₂-C₅)-alkynylene, where in each case one or two CH₂ group(s) may be replaced by O or NH;
Y CO, NHCO, SO, SO₂, or a bond;
R8, R9 independently of one another, hydrogen, SO₃H, sugar residue, (C₁-C₆)-alkyl, where one or more CH₂ groups of the alkyl radical may be substituted independently of one another by (C₁-C₆)-alkyl, OH, (C₁-C₆)-alkylene-OH, (C₂-C₆)-alkenylene-OH, O-sugar residue, OSO₃H, NH₂, NH—(C₁-C₆)-alkyl, N[(C₁-C₆)-alkyl]₂, NH—CO—(C₁-C₆)-alkyl, NH-sugar residue, NH—SO₃H, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂, (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl, adamantyl; or
R8 and R9 together with the N atom carrying them form a 5 to 7 membered, saturated ring Cyc2, where one or more CH₂ groups of the ring may also be replaced by O, S, NH, NSO₃H, N-sugar residue, N—(C₁-C₆)-alkyl, where one or more CH₂ groups of the alkyl radical may be substituted independently of one another by (C₁-C₆)-alkyl, OH, (C₁-C₆)-alkylene-OH, (C₂C₆)-alkenylene-OH, NH₂, NH—(C₁-C₆)-alkyl, N[(C₁-C₆)-alkyl]₂, NH—CO—(C₁-C₆)-alkyl, NH-sugar residue, (C₁-C₆)-alkylene-NH₂, (C₂-C₆)-alkenylene-NH₂, (C₀-C₆)-alkylene-COOH, (C₀-C₆)-alkylene-CONH₂, (C₀-C₆)-alkylene-CONH—(C₁-C₆)-alkyl, (C₀-C₆)-alkylene-SONH₂, (C₀-C₆)-alkylene-SONH—(C₁-C₆)-alkyl, (C₀-C₆)-alkylene-SO₂NH₂, (C₀-C₆)-alkylene-SO₂NH—(C₁-C₆)-alkyl;
and the pharmaceutically acceptable salts thereof;
for prevention and/or treatment of osteoporosis, osteolysis or aseptic loosening of joint implants.

7. The pharmaceutical composition of claim 2 comprising the compound of formula II 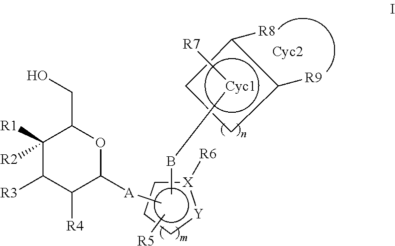
in which the meanings are
R1 and R2 independently of one another F, H or one of the radicals R1 or R2 OH;
R3 OH or F, where at least one of the radicals R1, R2, R3 must be F;
R4 OH;
A O, NH, CH₂, S or a bond;
X C, O, S or N, where X must be C when Y is O or S;
Y N, O or S;
m a number 1 or 2;
R5 hydrogen, F, Cl, Br, I, OH, CF₃, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO—(C₁-C₆)-alkyl, (C₁-C₆)-alkyl-O—(C₁-C₆)-alkyl, phenyl, benzyl, (C₁-C₆)-alkoxycarboxyl, it being possible for one, more than one or all hydrogen(s) in the alkyl, alkoxy, alkenyl or alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(C₁-C₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
or when Y is S, R5 and R6 together with the C atoms carrying them phenyl;
R6 optionally H, (C₁-C₆)-alkyl, (C₁-C₆)-alkenyl, (C₃-C₆)-cycloalkyl, or phenyl that may optionally be substituted by halogen or (C₁-C₄)-alkyl;
B (C₀-C₁₅)-alkanediyl, it being possible for one or more C atoms in the alkanediyl radical to be replaced independently of one another by —O—, —(C═O)—, —CH═CH—, —C≡C—, —S—, —CH(OH)—, —CHF—, —CF₂—, —(S═O)—, —(SO₂)—, —N(C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, HO—(C₁-C₆)-alkyl, (C₁-C₆)-alkyl-O—(C₁-C₆)-alkyl, it being possible for one, more than one or all hydrogen(s) in the alkyl, alkoxy, alkenyl or alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SCF₃, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO(C₁-C₆)-alkyl, CONH₂;
or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO(C₁-C₄)-alkyl, CONH₂, CONH(C₁-C₄)-alkyl, OCF₃;
and the pharmaceutically acceptable salts thereof
for prevention and/or treatment of osteoporosis, osteolysis or aseptic loosening of joint implants.

8. The pharmaceutical composition of claim 2 comprising the compound of formula III 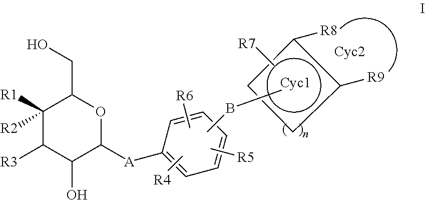
in which the meanings are
R1, R2 OH, F or H or R1 and R2=F, excluding the three combinations R1=F, R2=OH and R1=OH, R2=F and R1, R2=OH;
R3 OH or F, where at least one of the R1, R2, R3 radicals must be F;
A O, NH, CH₂, S or a bond;
R4, R5, R6 hydrogen, F, Cl, Br, I, OH, NO₂, CN, COOH, CO(C₁-C₆)-alkyl, COO(C₁-C₆)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₆)-alkoxy, HO(C₁-C₆)-alkyl, (C₁-C₆)-alkyl-O—(C₁-C₆)-alkyl, phenyl, benzyl, it being possible for one, more than one or all hydrogen(s) in the alkyl, alkoxy, alkenyl and alkynyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, O—(C₁-C₆)-alkyl, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
B (C₀-C₁₅)-alkanediyl, it being possible for one or more C atoms in the alkanediyl radical to be replaced independently of one another by —O—, —(C═O)—, —CH═CH—, —C≡C—, —S—, —CH(OH)—, —CHF—, —CF₂—, —(S═O)—; —(SO₂)—, —N((C₁-C₆)-alkyl)-, —N((C₁-C₆)-alkyl-phenyl)- or —NH—;
n a number from 0 to 4;
Cyc1 a 3 to 7 membered saturated, partially saturated or unsaturated ring, where 1 C atom may be replaced by O, N or S;
R7, R8, R9 hydrogen, F, Cl, Br, I, OH, CF₃, NO₂, CN, COOH, COO(C₁-C₆)-alkyl, CO(C₁-C₄)-alkyl, CONH₂, CONH(C₁-C₆)-alkyl, CON[(C₁-C₆)-alkyl]₂, (C₁-C₆)-alkyl, (C₂-C₆)-alkenyl, (C₂-C₆)-alkynyl, (C₁-C₈)-alkoxy, (C₁-C₆)-alkyl-OH, (C₁-C₆)-alkyl-O—(C₁-C₆)-alkyl, it being possible for one, more than one or all hydrogen(s) in the alkyl radicals to be replaced by fluorine;
SO₂—NH₂, SO₂NH(C₁-C₆)-alkyl, SO₂N[(C₁-C₆)-alkyl]₂, S—(C₁-C₆)-alkyl, S—(CH₂)ₒ-phenyl, SO—(C₁-C₆)-alkyl, SO—(CH₂)ₒ-phenyl, SO₂—(C₁-C₆)-alkyl, SO₂—(CH₂)ₒ-phenyl, where o can be 0-6, and the phenyl radical may be substituted up to twice by F, Cl, Br, OH, CF₃, NO₂, CN, OCF₃, O—(C₁-C₆)-alkyl, (C₁-C₆)-alkyl, NH₂;
NH₂, NH—(C₁-C₆)-alkyl, N((C₁-C₆)-alkyl)₂, NH(C₁-C₇)-acyl, phenyl, O—(CH₂)ₒ-phenyl, where o can be 0-6, where the phenyl ring may be substituted one to 3 times by F, Cl, Br, I, OH, CF₃, NO₂, CN, OCF₃, (C₁-C₈)-alkoxy, (C₁-C₆)-alkyl, NH₂, NH(C₁-C₆)-alkyl, N((C₁-C₆)-alkyl)₂, SO₂—CH₃, COOH, COO—(C₁-C₆)-alkyl, CONH₂;
or
R8 and R9 together with the C atoms carrying them a 5 to 7 membered, saturated, partially or completely unsaturated ring Cyc2, it being possible for 1 or 2 C atom(s) in the ring also to be replaced by N, O or S, and Cyc2 may optionally be substituted by (C₁-C₆)-alkyl, (C₂-C₅)-alkenyl, (C₂-C₅)-alkynyl, where in each case one CH₂ group may be replaced by O, or substituted by H, F, Cl, OH, CF₃, NO₂, CN, COO—(C₁-C₄)-alkyl, CONH₂, CONH(C₁-C₄)-alkyl, OCF₃;
and the pharmaceutically acceptable salts thereof
for the prevention and/or treatment of osteoporosis, osteolysis or aseptic loosening of joint implants.

9. (canceled)

10. (canceled)

11. A method for the treatment and/or prevention of osteoporosis, osteolysis and aseptic loosening of joint implants comprising the administration of the pharmaceutical composition of claim 2 to a patient in need thereof.
